(19) United States
(12) Patent Application Publication
(10) Pub. No.: US 2018/0030452 A1
(43) Pub. Date: Feb. 1, 2018

(54) TARGETING OLIGONUCLEOTIDES AND USES THEREOF TO MODULATE GENE EXPRESSION

(71) Applicant: Translate Bio MA, Inc., Cambridge, MA (US)

(72) Inventor: Fatih Ozsolak, Boston, MA (US)

(73) Assignee: Translate Bio MA, Inc., Cambridge, MA (US)

(21) Appl. No.: 15/550,103

(22) PCT Filed: Feb. 12, 2016
(86) PCT No.: PCT/US16/17773

§ 371 Date: Aug. 10, 2017

Related U.S. Application Data
(60) Provisional application No. 62/115,739, filed on Feb. 13, 2015.

(51) Int. Cl. C12N 15/113

(52) U.S. Cl. CPC C12N 15/1137; C12Y 116/03001; C12N 15/113; C12N 2310/321; C12N 2310/3231; C12N 2310/322; C12N 2310/343; C12N 2310/315; C12N 2310/351; C12N 2310/3513; C12N 2310/11

(57) ABSTRACT

Aspects of the invention provide single stranded oligonucleotides for modulating expression of genes based on targeting of low abundance non-coding RNA transcripts. Further aspects provide compositions and kits comprising single stranded oligonucleotides for modulating expression of genes. Methods for modulating expression of genes using the single stranded oligonucleotides are also provided. Further aspects of the invention provide methods for selecting a candidate oligonucleotide for modulating expression of genes.

### CROSS-REFERENCE TO RELATED APPLICATIONS

This application claims the benefit under 35 U.S.C. §119(e) of U.S. Provisional Application No. U.S. 62/115,739, entitled “TARGETING OLIGONUCLEOTIDES AND USES THEREOF TO MODULATE GENE EXPRESSION”, filed on Feb. 13, 2015, the contents of which are incorporated herein by reference in its entirety.

### FIELD OF THE INVENTION

The invention relates to oligonucleotide based compositions, as well as methods of using oligonucleotide based compositions for treating disease.

### BACKGROUND OF THE INVENTION

Modulation of gene expression is an important tool for basic research and for treating diseases caused by defective expression (either upregulation or downregulation) of one or more genes. Obtaining specificity with respect to modulation of a target gene as well as achieving sufficient modulation (e.g., sufficient upregulation or downregulation) to obtain a desired result, e.g., treatment of disease, remains a challenge. Additionally, limited approaches are available for increasing the expression of genes.

### SUMMARY OF THE INVENTION

Aspects of the invention disclosed herein provide methods and compositions that are useful for modulating (e.g., upregulating) expression of a target gene in cells. In some embodiments, single stranded oligonucleotides are provided that target a low-abundance non-coding RNA (lancRNA) of a target gene, e.g., encoding a protein of interest. In some embodiments, single stranded oligonucleotides are provided that target a lancRNA of a target gene (e.g., a human gene) and thereby cause modulation (e.g., upregulation) of the gene. In some embodiments, the target gene is a gene listed in Table 1. In some embodiments, these single stranded oligonucleotides modulate (e.g., activate or enhance) expression of a target gene by degrading the lancRNA or blocking the activity of the lancRNA. In some embodiments, these single stranded oligonucleotides modulate (e.g., activate or enhance) expression of a target gene to treat a disease or condition associated with reduced expression of the target gene. In some embodiments, the disease or condition associated with reduced expression of the target gene is listed is Table 2.

Further aspects of the invention provide methods for selecting oligonucleotides for modulating (e.g., activating or enhancing) expression of a target gene. In some embodiments, the target gene may be a target gene listed in Table 1, such as ABCA1, APOA1, ATP2A2, BDNF, FXN, HBA2, HBB, HBD, HBE1, HBG1, HBG2, SMN, UTRN, PTEN, MECP2, FOXP3, NFE2L2 (NRF2), THRB, NR1H4 (FXR), HAMP, ADIPOQ, PRKAA1, PRKAA2, PRKAB1, PRKAB2, PRKAG1, PRKAG2, or PRKAG3. In some embodiments, methods are provided for selecting a set of oligonucleotides that is enriched in candidates (e.g., compared with a random selection of oligonucleotides) for modulating (e.g., activating or enhancing) expression of a target gene. Accordingly, the methods may be used to establish sets of clinical candidates that are enriched in oligonucleotides that modulate (e.g., activate or enhance) expression of a target. Such libraries may be utilized, for example, to identify lead oligonucleotides for developing therapeutics to treat a disease or condition associated with reduced or enhanced expression of the target gene. In some embodiments, the disease or condition associated with reduced expression of the target gene is listed is Table 2 or otherwise disclosed herein. Furthermore, in some embodiments, oligonucleotide chemistries are provided that are useful for controlling the pharmacokinetics, biodistribution, bioavailability and/or efficacy of the single stranded oligonucleotides for modulating (e.g., activating) expression of a target gene.

In some aspects, a method of modulating expression of a target gene in cells is provided, the method comprising: delivering to the cells a single-stranded oligonucleotide of 8 to 50 nucleotides in length that comprises a region of complementarity that is complementary with at least 5 contiguous nucleotides of a low-abundance non-coding RNA (lancRNA) that modulates expression of a target gene in the cells, wherein the at least 5 contiguous nucleotides of the lancRNA are transcribed from a chromosomal region within 5 kb of a transcriptional boundary of the target gene.

In some embodiments, the lancRNA is represented at a level of less than 0.01 fragments per kilobase per million mapped reads (FPKM) based sequencing of RNA of the cells. In some embodiments, the lancRNA is represented at an average copy number of less than 10 (e.g., less than 0.1 or less than 0.0001) transcripts per cell. In some embodiments, the average copy number of the lancRNA is less than 1% of the average copy number of transcripts expressed from the target gene in the cells.

In some embodiments, the lancRNA is transcribed from the same strand of the chromosomal region as the target gene. In some embodiments, the lancRNA is transcribed from the opposite strand of the chromosomal region as the target gene.

In some embodiments, the at least 5 contiguous nucleotides of the lancRNA are transcribed from a chromosomal region within 5 kb (e.g., within 2 kb, within 1 kb, within 500 kb or within 250 bp) of a transcriptional boundary of the target gene.

In some embodiments, the transcriptional boundary is a transcriptional start site. In some embodiments, the transcriptional boundary is a transcriptional end site.

In some embodiments, the lancRNA is no more than 200 nucleotides in length.

In some embodiments, the target gene is ABCA1, APOA1, ATP2A2, BDNF, FXN, HBA2, HBB, HBD, HBE1, HBG1, HBG2, SMN, UTRN, PTEN, MECP2, FOXP3, NFE2L2 (NRF2), THRB, NR1H4 (FXR), HAMP, ADIPOQ, PRKAA1, PRKAA2, PRKAB1, PRKAB2, PRKAG1, PRKAG2, or PRKAG3. In some embodiments, the target gene is FXN.

In some embodiments, the oligonucleotide does not comprise three or more consecutive guanosine nucleotides. In some embodiments, the oligonucleotide does not comprise four or more consecutive guanosine nucleotides.

In some embodiments, the oligonucleotide is 8 to 30 nucleotides in length. In some embodiments, the oligonucleotide is 8 to 10 nucleotides in length and all but 1, 2, or 3 of the nucleotides of the complementary sequence of the lancRNA are cytosine or guanosine nucleotides.

In some embodiments, at least one nucleotide of the oligonucleotide is a nucleotide analogue. In some embodiments, the at least one nucleotide analogue results in an increase in Tm of the oligonucleotide in a range of 1 to 5° C. compared with an oligonucleotide that does not have the at least one nucleotide analogue.

In some embodiments, at least one nucleotide of the oligonucleotide comprises a 2′ O-methyl. In some embodiments, each nucleotide of the oligonucleotide comprises a 2′ O-methyl.

In some embodiments, wherein the oligonucleotide comprises at least one ribonucleotide, at least one deoxyribonucleotide, or at least one bridged nucleotide. In some embodiments, the bridged nucleotide is a LNA nucleotide, a cEt nucleotide or a ENA modified nucleotide. In some embodiments, each nucleotide of the oligonucleotide is a LNA nucleotide.

In some embodiments, the nucleotides of the oligonucleotide comprise alternating deoxyribonucleotides and 2′-fluoro-deoxyribonucleotides. In some embodiments, the nucleotides of the oligonucleotide comprise alternating deoxyribonucleotides and 2′-O-methyl nucleotides. In some embodiments, the nucleotides of the oligonucleotide comprise alternating deoxyribonucleotides and ENA nucleotide analogues. In some embodiments, the nucleotides of the oligonucleotide comprise alternating deoxyribonucleotides and LNA nucleotides. In some embodiments, the 5′ nucleotide of the oligonucleotide is a deoxyribonucleotide.

In some embodiments, the nucleotides of the oligonucleotide comprise alternating LNA nucleotides and 2′-O-methyl nucleotides. In some embodiments, the 5′ nucleotide of the oligonucleotide is a LNA nucleotide.

In some embodiments, the nucleotides of the oligonucleotide comprise deoxyribonucleotides flanked by at least one LNA nucleotide on each of the 5′ and 3′ ends of the deoxyribonucleotides.

In some embodiments, the oligonucleotide further comprises phosphorothioate internucleotide linkages between at least two nucleotides. In some embodiments, the oligonucleotide further comprises phosphorothioate internucleotide linkages between all nucleotides.

In some embodiments, the nucleotide at the 3′ position of the oligonucleotide has a 3′ hydroxyl group. In some embodiments, the nucleotide at the 3′ position of the oligonucleotide has a 3′ thiophosphate.

In some embodiments, the oligonucleotide further comprises a biotin moiety conjugated to the 5′ nucleotide.

In some embodiments, the oligonucleotide comprises a nucleotide sequence as set for in Table 3.

Other aspects provide a single stranded oligonucleotide having a nucleotide sequence as set forth in Table 3.

In some embodiments, at least one nucleotide of the oligonucleotide comprises a 2′ O-methyl. In some embodiments, each nucleotide of the oligonucleotide comprises a 2′ O-methyl.

In some embodiments, the oligonucleotide comprises at least one ribonucleotide, at least one deoxyribonucleotide, or at least one bridged nucleotide. In some embodiments, the bridged nucleotide is a LNA nucleotide, a cEt nucleotide or a ENA modified nucleotide. In some embodiments, each nucleotide of the oligonucleotide is a LNA nucleotide.

In some embodiments, the nucleotides of the oligonucleotide comprise alternating deoxyribonucleotides and 2′-fluoro-deoxyribonucleotides. In some embodiments, the nucleotides of the oligonucleotide comprise alternating deoxyribonucleotides and 2′-O-methyl nucleotides. In some embodiments, the nucleotides of the oligonucleotide comprise alternating deoxyribonucleotides and ENA nucleotide analogues. In some embodiments, the nucleotides of the oligonucleotide comprise alternating deoxyribonucleotides and LNA nucleotides. In some embodiments, the 5′ nucleotide of the oligonucleotide is a deoxyribonucleotide.

In some embodiments, the nucleotides of the oligonucleotide comprise alternating LNA nucleotides and 2′-O-methyl nucleotides. In some embodiments, the 5′ nucleotide of the oligonucleotide is a LNA nucleotide.

In some embodiments, the nucleotides of the oligonucleotide comprise deoxyribonucleotides flanked by at least one LNA nucleotide on each of the 5′ and 3′ ends of the deoxyribonucleotides.

In some embodiments, the oligonucleotide further comprises phosphorothioate internucleotide linkages between at least two nucleotides.

In some embodiments, the oligonucleotide further comprises phosphorothioate internucleotide linkages between all nucleotides.

In some embodiments, the nucleotide at the 3′ position of the oligonucleotide has a 3′ hydroxyl group.

In some embodiments, the nucleotide at the 3′ position of the oligonucleotide has a 3′ thiophosphate.

In some embodiments, the oligonucleotide further comprises a biotin moiety conjugated to the 5′ nucleotide.

In some embodiments, the modification pattern for the oligonucleotide is the modification pattern provided in Table 3.

Other aspects relate to a composition comprising a single stranded oligonucleotide as described herein, such as in any embodiment described above, and a carrier. In some embodiments, the carrier is a peptide. In some embodiments, the carrier is a steroid. In some embodiments, the oligonucleotide is conjugated to the carrier.

Yet other aspects relate to a composition comprising a single stranded oligonucleotide as described herein, such as in any embodiment described above, in a buffered solution.

In another aspect a pharmaceutical composition is provided comprising a composition as described herein, such as in any embodiment described above, and a pharmaceutically acceptable carrier.

In yet another aspect a kit is provided comprising a container housing a composition as described herein, such as in any embodiment described above.

In other aspects, a method of modulating expression of a target gene in cells is provided, the method comprising:

i) determining presence of a low-abundance non-coding RNA (lancRNA) in cells; and

ii) based on the determination made in i), delivering to the cells a single-stranded oligonucleotide of 8 to 50 nucleotides in length that comprises a region of complementarity that is complementary with at least 5 contiguous nucleotides of a lancRNA that modulates expression of a target gene in the cells, wherein the at least 5 contiguous nucleotides of the lancRNA are transcribed from a chromosomal region within 5 kb of a transcriptional boundary of the target gene.

In some embodiments, in step i) the lancRNA is determined to be present at a level of less than 0.01 fragments per kilobase per million mapped reads (FPKM) based sequencing of RNA of the cells. In some embodiments, in step i) the lancRNA is determined to be present at an average copy number of less than 10 (e.g., less than 0.1 or less than 0.0001) transcripts per cell. In some embodiments, in step i) the lancRNA is determined to be present at less than 1% of the average copy number of transcripts expressed from the target gene in the cells.

In another aspect, a method of modulating expression of a target gene in cells is provided, the method comprising: delivering to the cells a single-stranded oligonucleotide of 8 to 50 nucleotides in length that comprises a region of complementarity that is complementary with at least 5 contiguous nucleotides of a chromosomal region that corresponds to a 3′ UTR of the target gene, wherein the at least 5 contiguous nucleotides are antisense to the target gene. In some embodiments, the method comprises delivering to the cells a single-stranded oligonucleotide of 8 to 50 nucleotides in length that comprises a region of complementarity that is complementary with at least 5 contiguous nucleotides of a chromosomal region that encodes a 3′ UTR of the target gene, wherein the at least 5 contiguous nucleotides are on the opposite strand of the chromosomal region as the target gene.

In some embodiments, the target gene is ABCA1, APOA1, ATP2A2, BDNF, FXN, HBA2, HBB, HBD, HBE1, HBG1, HBG2, SMN, UTRN, PTEN, MECP2, FOXP3, NFE2L2 (NRF2), THRB, NR1H4 (FXR), HAMP, ADIPOQ, PRKAA1, PRKAA2, PRKAB1, PRKAB2, PRKAG1, PRKAG2, or PRKAG3. In some embodiments, the target gene is FXN.

In some embodiments, the oligonucleotide does not comprise three or more consecutive guanosine nucleotides. In some embodiments, the oligonucleotide does not comprise four or more consecutive guanosine nucleotides.

In some embodiments, the oligonucleotide is 8 to 30 nucleotides in length. In some embodiments, the oligonucleotide is 8 to 10 nucleotides in length and all but 1, 2, or 3 of the nucleotides of the complementary sequence of the lancRNA are cytosine or guanosine nucleotides.

In some embodiments, at least one nucleotide of the oligonucleotide is a nucleotide analogue. In some embodiments, the at least one nucleotide analogue results in an increase in Tm of the oligonucleotide in a range of 1 to 5° C. compared with an oligonucleotide that does not have the at least one nucleotide analogue. In some embodiments, at least one nucleotide of the oligonucleotide comprises a 2′ O-methyl. In some embodiments, the oligonucleotide comprises at least one ribonucleotide, at least one deoxyribonucleotide, or at least one bridged nucleotide. In some embodiments, the bridged nucleotide is a LNA nucleotide, a cEt nucleotide or a ENA modified nucleotide.

In some embodiments, the oligonucleotide further comprises phosphorothioate internucleotide linkages between at least two nucleotides. In some embodiments, the oligonucleotide further comprises phosphorothioate internucleotide linkages between all nucleotides.

In some embodiments, the nucleotide at the 3′ position of the oligonucleotide has a 3′ hydroxyl group. In some embodiments, the nucleotide at the 3′ position of the oligonucleotide has a 3′ thiophosphate.

In some embodiments, a single stranded oligonucleotide provided herein comprises a fragment of at least 8 nucleotides of a nucleotide sequence as set forth in Table 3. In some embodiments, the single stranded oligonucleotide comprises or consists of a nucleotide sequence as set forth in Table 3. In some embodiments, the single stranded oligonucleotide comprises or consists of a modification pattern as set forth in Table 3. In some embodiments, one or more sequences in Table 3 are excluded, e.g., FXN-375, FXN-390, FXN-577, and FXN-578 in Table 3 are excluded.

In some embodiments, the single stranded oligonucleotide does not comprise three or more consecutive guanosine nucleotides. In some embodiments, the single stranded oligonucleotide does not comprise four or more consecutive guanosine nucleotides.

In some embodiments, the single stranded oligonucleotide is 8 to 30 nucleotides in length. In some embodiments, the single stranded oligonucleotide is up to 50 nucleotides in length. In some embodiments, the single stranded oligonucleotide is 8 to 10 nucleotides in length and all but 1, 2, or 3 of the nucleotides of the complementary sequence of the lancRNA are cytosine or guanosine nucleotides.

In some embodiments, the single stranded oligonucleotide is complementary with at least 8 consecutive nucleotides of a lancRNA of a target gene, in which the nucleotide sequence of the single stranded oligonucleotide comprises one or more of a nucleotide sequence selected from the group consisting of

(a) (X)Xxxxxx, (X)xXxxxx, (X)xxXxxx, (X)xxxXxx, (X)xxxxXx and (X)xxxxxX,

(b) (X)XXxxxx, (X)XxXxxx, (X)XxxXxx, (X)XxxxXx, (X)XxxxxX, (X)xXXxxx, (X)xXxXxx, (X)xXxxXx, (X)xXxxxX, (X)xxXXxx, (X)xxXxXx, (X)xxXxxX, (X)xxxXXx, (X)xxxXxX and (X)xxxxXX,

(c) (X)XXXxxx, (X)xXXXxx, (X)xxXXXx, (X)xxxXXX, (X)XXxXxx, (X)XXxxXx, (X)XXxxxX, (X)xXXxXx, (X)xXXxxX, (X)xxXXxX, (X)XxXXxx, (X)XxxXXx (X)XxxxXX, (X)xXxXXx, (X)xXxxXX, (X)xxXxXX, (X)xXxXxX and (X)XxXxXx,

(d) (X)xxXXX, (X)xXxXXX, (X)xXXxXX, (X)xXXXxX, (X)xXXXXx, (X)XxxXXXX, (X)XxXxXX, (X)XxXXxX, (X)XxXXx, (X)XXxxXX, (X)XXxXxX, (X)XXxXXx, (X)XXXxxX, (X)XXXxXx, and (X)XXXXxx,

(e) (X)xXXXXX, (X)XxXXXX, (X)XXxXXX, (X)XXXxXX, (X)XXXXxX and (X)XXXXXx, and

(f) XXXXXX, XxXXXXX, XXxXXXX, XXXxXXX, XXXXxXX, XXXXXxX and XXXXXXx, wherein “X” denotes a nucleotide analogue, (X) denotes an optional nucleotide analogue, and “x” denotes a DNA or RNA nucleotide unit.

In some embodiments, at least one nucleotide of the oligonucleotide is a nucleotide analogue. In some embodiments, the at least one nucleotide analogue results in an increase in Tm of the oligonucleotide in a range of 1 to 5° C. compared with an oligonucleotide that does not have the at least one nucleotide analogue.

In some embodiments, at least one nucleotide of the oligonucleotide comprises a 2′ O-methyl. In some embodiments, each nucleotide of the oligonucleotide comprises a 2′ O-methyl. In some embodiments, the oligonucleotide comprises at least one ribonucleotide, at least one deoxyribonucleotide, or at least one bridged nucleotide. In some embodiments, the bridged nucleotide is a LNA nucleotide, a cEt nucleotide or a ENA modified nucleotide. In some embodiments, each nucleotide of the oligonucleotide is a LNA nucleotide.

In some embodiments, the nucleotides of the oligonucleotide comprise alternating deoxyribonucleotides and 2′-fluoro-deoxyribonucleotides. In some embodiments, the nucleotides of the oligonucleotide comprise alternating deoxyribonucleotides and 2′-O-methyl nucleotides. In some embodiments, the nucleotides of the oligonucleotide comprise alternating deoxyribonucleotides and ENA nucleotide analogues. In some embodiments, the nucleotides of the oligonucleotide comprise alternating deoxyribonucleotides and LNA nucleotides. In some embodiments, the 5′ nucleotide of the oligonucleotide is a deoxyribonucleotide. In some embodiments, the nucleotides of the oligonucleotide comprise alternating LNA nucleotides and 2′-O-methyl nucleotides. In some embodiments, the 5′ nucleotide of the oligonucleotide is a LNA nucleotide. In some embodiments, the nucleotides of the oligonucleotide comprise deoxyribonucleotides flanked by at least one LNA nucleotide on each of the 5′ and 3′ ends of the deoxyribonucleotides.

In some embodiments, the single stranded oligonucleotide comprises modified internucleotide linkages (e.g., phosphorothioate internucleotide linkages or other linkages) between at least two, at least three, at least four, at least five or more nucleotides. In some embodiments, the single stranded oligonucleotide comprises modified internucleotide linkages (e.g., phosphorothioate internucleotide linkages or other linkages) between all nucleotides.

In some embodiments, the nucleotide at the 3′ position of the oligonucleotide has a 3′ hydroxyl group. In some embodiments, the nucleotide at the 3′ position of the oligonucleotide has a 3′ thiophosphate. In some embodiments, the single stranded oligonucleotide has a biotin moiety or other moiety conjugated to its 5′ or 3′ nucleotide. In some embodiments, the single stranded oligonucleotide has cholesterol, Vitamin A, folate, sigma receptor ligands, aptamers, peptides, such as CPP, hydrophobic molecules, such as lipids, ASGPR or dynamic polyconjugates and variants thereof at its 5′ or 3′ end.

According to some aspects of the invention compositions are provided that comprise any of the oligonucleotides disclosed herein, and a carrier. In some embodiments, compositions are provided that comprise any of the oligonucleotides in a buffered solution. In some embodiments, the oligonucleotide is conjugated to the carrier. In some embodiments, the carrier is a peptide. In some embodiments, the carrier is a steroid. According to some aspects of the invention pharmaceutical compositions are provided that comprise any of the oligonucleotides disclosed herein, and a pharmaceutically acceptable carrier.

According to other aspects of the invention, kits are provided that comprise a container housing any of the compositions disclosed herein.

According to some aspects of the invention, methods of modulatin (e.g., increasing) expression of a target gene in a cell are provided. In some embodiments, the methods involve delivering any one or more of the single stranded oligonucleotides disclosed herein into the cell. In some embodiments, delivery of the single stranded oligonucleotide into the cell results in a level of expression of the target gene that is greater (e.g., at least 50% greater) than a level of expression of the target gene in a control cell that does not comprise the single stranded oligonucleotide.

According to some aspects of the invention, methods of increasing levels of a target gene in a subject are provided. According to some aspects of the invention, methods of treating a disease or condition (e.g., a disease or condition provided in Table 2) associated with decreased levels of a target gene in a subject are provided. In some embodiments, the methods involve administering any one or more of the single stranded oligonucleotides disclosed herein to the subject.

### BRIEF DESCRIPTION OF THE DRAWINGS

FIG. 1 is a diagram showing the APOA1 gene locus with oligos targeting 5′/3′ antisense regions encoding lancRNAs.

FIG. 2A is a diagram FXN gene locus with oligos targeting 3′ antisense regions encoding lancRNAs.

FIG. 2B is a diagram FXN gene locus with oligos targeting 5′ antisense regions encoding lancRNAs. The sequences correspond to SEQ ID NO: 296.

FIG. 3A is a graph showing APOA1 mRNA levels in cells treated with APOA1 oligos (oligos are those shown in Table 3, “26”=Apoa1_mus-26, “27”=Apoa1_mus-27, etc. in Table 3).

FIG. 3B is a graph showing APOA1 mRNA levels in cells treated with APOA1 oligos (oligos are those shown in Table 3, “33”=Apoa1_mus-33, “34”=Apoa1_mus-34, etc. in Table 3).

FIG. 3C is a graph showing APOA1 mRNA levels in cells treated with APOA1 oligos (oligos are those shown in Table 3, “40”=Apoa1_mus-40, “41”=Apoa1_mus-41, etc. in Table 3).

FIG. 4A is a photograph of a Western blot showing APOA1 protein levels in cells treated with APOA1 oligos (oligos are those shown in Table 3, “26”=Apoa1_mus-26, “27”=Apoa1_mus-27, etc. in Table 3).

FIG. 4B is a photograph of a Western blot showing APOA1 protein levels in cells treated with APOA1 oligos (oligos are those shown in Table 3, “33”=Apoa1_mus-33, “34”=Apoa1_mus-34, etc. in Table 3).

FIG. 4C is a photograph of a Western blot showing APOA1 protein levels in cells treated with APOA1 oligos (oligos are those shown in Table 3, “40”=Apoa1_mus-40, “41”=Apoa1_mus-41, etc. in Table 3).

FIG. 5 is a graph showing FXN mRNA levels is Sarsero fibroblasts treated with FXN oligos (oligos are those shown in Table 3, “606”=FXN-606, “607”=FXN-607, etc. in Table 3). The oligo names on the X-axis are, from left to right, 606-653 in numerical order.

FIG. 6 is a graph showing FXN mRNA levels is GM03816 cells treated with FXN oligos (oligos are those shown in Table 3, “606”=FXN-606, “607”=FXN-607, etc. in Table 3). The oligo names on the X-axis are, from left to right, 606-653 in numerical order.

FIG. 7 is a graph showing FXN mRNA levels is GM03816 cells treated with FXN oligos (oligos are those shown in Table 3, “800”=FXN-800, “801”=FXN-801, etc. in Table 3). For each oligo on the X-axis, the concentrations are, from left to right, 50 nM, 25 nM, 12.5 nM, 6.25 nM, 3.125 nM, or water. The oligo names on the X-axis are, from left to right, 800-804 in numerical order, 800-812 in numerical order, 588, 594, 40, 823-827 in numerical order, and 816-822 in numerical order.

FIG. 8 is a photograph of a Western blot showing FXN protein levels in Sarsero cells treated with FXN oligos (oligos are those shown in Table 3, “606”=FXN-606, “607”=FXN-607, etc. in Table 3).

FIG. 9 is a photograph of a Western blot showing FXN protein levels in GM03816 cells treated with FXN oligos (oligos are those shown in Table 3, “606”=FXN-606, “607”=FXN-607, etc. in Table 3).

FIG. 10A is a graph showing FXN mRNA levels is cardiomyocytes treated with FXN oligos (oligos are those shown in Table 3, “603”=FXN-603, “62”=FXN-62, etc. in Table 3).

FIG. 10B is a graph showing FXN mRNA levels is cardiomyocytes treated with FXN oligos (oligos are those shown in Table 3, “634”=FXN-634, “643”=FXN-643, etc. in Table 3).

FIG. 10C is a photograph of a Western blot showing FXN protein levels is cardiomyocytes treated with FXN oligos (oligos are those shown in Table 3, “603”=FXN-603, “607”=FXN-607, etc. in Table 3).

FIG. 11A is a graph showing FXN mRNA levels in a liver from a mouse treated with FXN oligos (oligos are those shown in Table 3, “603”=FXN-603, “607”=FXN-607, etc. in Table 3).

FIG. 11B is a graph showing FXN mRNA levels in a liver from a mouse treated with FXN oligos (oligos are those shown in Table 3, “643”=FXN-643, etc. in Table 3).

FIG. 11C is a graph showing FXN total mRNA levels a mouse treated with FXN oligos (oligos are those shown in Table 3, “603”=FXN-603, “607”=FXN-607, etc. in Table 3).

FIG. 11D is a graph showing FXN total mRNA levels a mouse treated with FXN oligos (oligos are those shown in Table 3, “643”=FXN-643, etc. in Table 3).

### DETAILED DESCRIPTION OF CERTAIN EMBODIMENTS OF THE INVENTION

Aspects of the invention relate to modulation of gene expression. A considerable portion of human diseases can be treated by selectively altering protein and/or RNA levels of disease-associated transcription units (noncoding short and long RNAs, protein-coding RNAs or other regulatory coding or noncoding genomic regions).

Genomic regions encoding main RNA transcript units (e.g., genes) also produce RNA species such as PARs (promoter-associated RNAs) and TARs (termini-associated RNAs), which are a class of short (e.g., <200 nucleotides) or long noncoding RNAs expressed at low abundance at or near the 5′ and 3′ end of genes. The gene may be a protein coding gene or a gene that encodes a noncoding RNA. The low abundance noncoding RNAs (lancRNAs) from these regions can be both in sense or antisense orientation to the main transcript being produced. As described herein, single stranded oligonucleotides were designed to be complementary to chromosomal regions encoding lancRNAs, thereby targeting the lancRNAs. It was found that gene expression was modulated after administration of these oligonucleotides to cells, resulting in many instances in upregulation of genes tested (e.g., APOA1, FXN). Without wishing to be bound by theory, it is thought that targeting these lancRNAs resulted in modulation of gene expression. Again, without wishing to be bound by theory, the regulation of parent RNA behavior through these lancRNAs can be through various mechanisms, including, but not limited to, transcriptional mechanisms, splicing mechanisms, posttranscriptional mechanisms and mechanisms affecting translation efficiency and levels. The chromosomal regions containing these lancRNAs can be +/−200 nucleotides, +/−500 nucleotides, +/−1000 nucleotides, +/−5000 nucleotides, or more, of transcriptional boundaries (e.g., 5′ and 3′ ends) of genes. As used herein, the term “low abundance noncoding RNA (lancRNA)” has its common meaning in the art and generally refers to a non-coding RNA that is present at low levels in cells, for example, at levels of less than 50 transcripts per cell. In some embodiments, a low abundance noncoding RNA has a copy number (e.g., average copy number) in a population of appropriate cells of less than 50, less than 40, less than 30, less than 20, less than 10, less than 5, less than 3, less than 1, less than 0.1, less than 0.01, less than 0.001 or less than 0.0001 transcripts per cell in the population. In some embodiments, a low abundance noncoding RNA is at a level of less than 100, less than 50, less than 40, less than 30, less than 20, less than 10, less than 1, less than 0.1, less than 0.01, less than 0.001, less than 0.0001 fragments per kilobase per million mapped reads (FPKM) based sequencing of RNA obtained from cells of an appropriate cell population. In some embodiments, a low abundance noncoding RNA is at a level of less than 100, less than 50, less than 40, less than 30, less than 20, less than 10, less than 1, less than 0.1, less than 0.01, less than 0.001, less than 0.0001 reads per kilobase per million mapped reads (RPKM) based sequencing of RNA obtained from cells of an appropriate cell population. In some embodiments, a low abundance noncoding RNA has a copy number (e.g., an average copy number) in a population of appropriate cells of less than 50%, less than 40%, less than 30%, less than 20%, less than 10%, less than 5%, less than 1%, less than 0.5%, less than 0.05%, less than 0.01% or less than 0.001%, of the average copy number of transcripts expressed from a target gene of the low abundance noncoding RNA in cells of the population. Methods for calculating FPKM, RPKM, and copy number are well known in the art (see, e.g., Hart et al. Finding the active genes in deep RNA-seq gene expression studies. BMC Genomics. 2013 Nov. 11; 14:778; and Trapnell et al. Transcript assembly and quantification by RNA-Seq reveals unannotated transcripts and isoform switching during cell differentiation. Nat Biotechnol. 2010 May; 28(5):511-5).

In some embodiments, the lancRNA has a length of no more than 1000, 500, 400, 300, or 200 nucleotides. In some embodiments, the lancRNA has a length of between 10 and 1000 nucleotides, 10 and 500 nucleotides, 10 and 400 nucleotides, 10 and 300 nucleotides, 10 and 200 nucleotides, 50 and 1000 nucleotides, 50 and 500 nucleotides, 50 and 400 nucleotides, 50 and 300 nucleotides, 50 and 200 nucleotides, 100 and 1000 nucleotides, 100 and 500 nucleotides, 100 and 400 nucleotides, 100 and 300 nucleotides, or 100 and 200 nucleotides.

In some embodiments, single stranded oligonucleotides are provided that specifically bind to, or are complementary to, a lancRNA transcribed from a genomic region that is within, spans or is in proximity to a target gene. In some embodiments, single stranded oligonucleotides are provided that specifically bind to, or are complementary to, a lancRNA that is transcribed from a chromosomal region that encompasses +/−100 nucleotides, +/−200 nucleotides, +/−300 nucleotides, +/−400 nucleotides, +/−500 nucleotides, +/−600 nucleotides, +/−700 nucleotides, +/−800 nucleotides, +/−900 nucleotides, +/−1000 nucleotides, +/−2000 nucleotides, +/−3000 nucleotides, +/−4000 nucleotides, +/−5000 nucleotides, or more, of a 5′ or 3′ end of a target gene. The lancRNA may be transcribed from the strand that is antisense to the target gene or sense to the target gene.

Accordingly, in some aspects the invention contemplates single stranded oligonucleotides that specifically bind to, or are complementary to, a sense strand or antisense strand of a chromosomal region that encompasses +/−100 nucleotides, +/−200 nucleotides, +/−300 nucleotides, +/−400 nucleotides, +/−500 nucleotides, +/−600 nucleotides, +/−700 nucleotides, +/−800 nucleotides, +/−900 nucleotides, +/−1000 nucleotides, +/−2000 nucleotides, +/−3000 nucleotides, +/−4000 nucleotides, +/−5000 nucleotides, or more, of a transcriptional boundary (e.g., a 5′ or 3′ end) of a target gene. In some embodiments, the single stranded oligonucleotide specifically binds to, or is complementary to, a region of an antisense strand (relative to the target gene) within a chromosomal region that encodes a 3′UTR of the target gene. In some embodiments, the single stranded oligonucleotide specifically binds to, or is complementary to, a region of a sense strand (relative to the target gene) within a chromosomal region that encodes a 3′UTR of the target gene. In some embodiments, the single stranded oligonucleotide specifically binds to, or is complementary to, a region of an antisense strand (relative to the target gene) within a chromosomal region that encodes a 5′UTR of the target gene. In some embodiments, the single stranded oligonucleotide specifically binds to, or is complementary to, a region of a sense strand (relative to the target gene) within a chromosomal region that encodes a 5′UTR of the target gene.

Methods for identifying transcript ends (e.g., transcriptional start sites and polyadenylation junctions) are known in the art and may be used in selecting oligonucleotides that specifically bind to lancRNAs transcribed from chromosomal regions encompassing these ends. In some embodiments, 3′ end oligonucleotides may be designed by identifying RNA 3′ ends (also referred to herein as transcription end sites) using quantitative end analysis of poly-A tails, designating a window (e.g., 200 nucleotides, 500 nucleotides, 1000 nucleotides, 2000 nucleotides, 5000 nucleotides, or more) that encompasses the 3′ end, and designing oligonucleotides that are complementary to either the sense or antisense strand relative to the target gene within the designated window. In some embodiments, 5′ end oligonucleotides may be designed by identifying 5′ start sites (also referred to herein as transcriptional start sites) using Cap analysis gene expression (CAGE), designating a window (e.g., 200 nucleotides, 500 nucleotides, 1000 nucleotides, 2000 nucleotides, 5000 nucleotides, or more) that encompasses the 5′ start site, and designing oligonucleotides that are complementary to either the sense or antisense strand relative to the target gene within the designated window. Appropriate methods are disclosed, for example, in Ozsolak et al. Comprehensive Polyadenylation Site Maps in Yeast and Human Reveal Pervasive Alternative Polyadenylation. Cell. Volume 143, Issue 6, 2010, Pages 1018-1029; Shiraki, T, et al., Cap analysis gene expression for high-throughput analysis of transcriptional starting point and identification of promoter usage. Proc Natl Acad Sci USA. 100 (26): 15776-81. 2003 Dec. 23; and Zhao, X, et al., (2011). Systematic Clustering of Transcription Start Site Landscapes. PLoS ONE (Public Library of Science) 6 (8): e23409, the contents of each of which are incorporated herein by reference. Other appropriate methods for identifying transcript start sites and polyadenylation junctions may also be used, including, for example, RNA-Paired-end tags (PET) (See, e.g., Ruan X, Ruan Y. Methods Mol Biol. 2012; 809:535-62); use of standard EST databases; RACE combined with microarray or sequencing, PAS-Seq (See, e.g., Peter J. Shepard, et al., RNA. 2011 April; 17(4): 761-772); and 3P-Seq (See, e.g., Calvin H. Jan, Nature. 2011 Jan. 6; 469(7328): 97-101; and others.

In some embodiments, the target gene is a gene provided in Table 1. In some embodiments, the transcriptional boundaries of the target gene refer to the 5′ and 3′ end of the exemplary mRNA provided in Table 1 for the target gene.

TABLE 1
Non-limiting examples of RNA transcripts for certain genes

| GENE | | | |
|---|---|---|---|
| SYMBOL | MRNA | SPECIES | GENE NAME |
| ABCA1 | NM_013454 | Mus | ATP-binding cassette, sub-family A (ABC1), |
| | | musculus | member 1 |
| ABCA1 | NM_005502 | Homo | ATP-binding cassette, sub-family A (ABC1), |
| | | sapiens | member 1 |
| ABCA4 | NM_007378 | Mus | ATP-binding cassette, sub-family A (ABC1), |
| | | musculus | member 4 |
| ABCA4 | NM_000350 | Homo | ATP-binding cassette, sub-family A (ABC1), |
| | | sapiens | member 4 |
| ABCB11 | NM_003742 | Homo | ATP-binding cassette, sub-family B |
| | | sapiens | (MDR/TAP), member 11 |
| ABCB11 | NM_021022 | Mus | ATP-binding cassette, sub-family B |
| | | musculus | (MDR/TAP), member 11 |
| ABCB4 | NM_018850 | Homo | ATP-binding cassette, sub-family B |
| | | sapiens | (MDR/TAP), member 4 |
| ABCB4 | NM_000443 | Homo | ATP-binding cassette, sub-family B |
| | | sapiens | (MDR/TAP), member 4 |
| ABCB4 | NM_018849 | Homo | ATP-binding cassette, sub-family B |
| | | sapiens | (MDR/TAP), member 4 |
| ABCB4 | NM_008830 | Mus | ATP-binding cassette, sub-family B |
| | | musculus | (MDR/TAP), member 4 |
| ABCG5 | NM_022436 | Homo | ATP-binding cassette, sub-family G (WHITE), |
| | | sapiens | member 5 |
| ABCG5 | NM_031884 | Mus | ATP-binding cassette, sub-family G (WHITE), |
| | | musculus | member 5 |
| ABCG8 | NM_026180 | Mus | ATP-binding cassette, sub-family G (WHITE), |
| | | musculus | member 8 |
| ABCG8 | NM_022437 | Homo | ATP-binding cassette, sub-family G (WHITE), |
| | | sapiens | member 8 |
| ADIPOQ | NM_009605 | Mus | adiponectin, C1Q and collagen domain |
| | | musculus | containing |
| ADIPOQ | NM_004797 | Homo | adiponectin, C1Q and collagen domain |
| | | sapiens | containing |
| ALB | NM_000477 | Homo | albumin |
| | | sapiens | |
| ALB | NM_009654 | Mus | albumin |
| | | musculus | |
| APOA1 | NM_000039 | Homo | apolipoprotein A-I |
| | | sapiens | |
| APOA1 | NM_009692 | Mus | apolipoprotein A-l |
| | | musculus | |
| APOE | NM_009696 | Mus | apolipoprotein E |
| | | musculus | |
| APOE | XM_001724655 | Homo | hypothetical LOC100129500; |
| | | sapiens | apolipoprotein E |
| APOE | XM_001722911 | Homo | hypothetical LOC100129500; |
| | | sapiens | apolipoprotein E |
| APOE | XM_001724653 | Homo | hypothetical LOC100129500; |
| | | sapiens | apolipoprotein E |
| APOE | NM_000041 | Homo | hypothetical LOC100129500; |
| | | sapiens | apolipoprotein E |
| APOE | XM_001722946 | Homo | hypothetical LOC100129500; |
| | | sapiens | apolipoprotein E |
| ATP2A2 | NM_009722 | Mus | ATPase, Ca++ transporting, cardiac muscle, |
| | | musculus | slow twitch 2 |
| ATP2A2 | NM_001110140 | Mus | ATPase, Ca++ transporting, cardiac muscle, |
| | | musculus | slow twitch 2 |
| ATP2A2 | NM_001135765 | Homo | ATPase, Ca++ transporting, cardiac muscle, |
| | | sapiens | slow twitch 2 |
| ATP2A2 | NM_170665 | Homo | ATPase, Ca++ transporting, cardiac muscle, |
| | | sapiens | slow twitch 2 |
| ATP2A2 | NM_001681 | Homo | ATPase, Ca++ transporting, cardiac muscle, |
| | | sapiens | slow twitch 2 |
| BCL2L11 | NM_006538 | Homo | BCL2-like 11 (apoptosis facilitator) |
| | | sapiens | |
| BCL2L11 | NM_207002 | Homo | BCL2-like 11 (apoptosis facilitator) |
| | | sapiens | |
| BCL2L11 | NM_138621 | Homo | BCL2-like 11 (apoptosis facilitator) |
| | | sapiens | |
| BCL2L11 | NM_207680 | Mus | BCL2-like 11 (apoptosis facilitator) |
| | | musculus | |
| BCL2L11 | NM_207681 | Mus | BCL2-like 11 (apoptosis facilitator) |
| | | musculus | |
| BCL2L11 | NM_009754 | Mus | BCL2-like 11 (apoptosis facilitator) |
| | | musculus | |
| BDNF | NM_001143816 | Homo | brain-derived neurotrophic factor |
| | | sapiens | |
| BDNF | NM_001143815 | Homo | brain-derived neurotrophic factor |
| | | sapiens | |
| BDNF | NM_001143814 | Homo | brain-derived neurotrophic factor |
| | | sapiens | |
| BDNF | NM_001143813 | Homo | brain-derived neurotrophic factor |
| | | sapiens | |
| BDNF | NM_001143812 | Homo | brain-derived neurotrophic factor |
| | | sapiens | |
| BDNF | NM_001143806 | Homo | brain-derived neurotrophic factor |
| | | sapiens | |
| BDNF | NM_001143811 | Homo | brain-derived neurotrophic factor |
| | | sapiens | |
| BDNF | NM_001143805 | Homo | brain-derived neurotrophic factor |
| | | sapiens | |
| BDNF | NM_001143810 | Homo | brain-derived neurotrophic factor |
| | | sapiens | |
| BDNF | NM_001709 | Homo | brain-derived neurotrophic factor |
| | | sapiens | |
| BDNF | NM_170735 | Homo | brain-derived neurotrophic factor |
| | | sapiens | |
| BDNF | NM_170734 | Homo | brain-derived neurotrophic factor |
| | | sapiens | |
| BDNF | NM_170733 | Homo | brain-derived neurotrophic factor |
| | | sapiens | |
| BDNF | NM_170732 | Homo | brain-derived neurotrophic factor |
| | | sapiens | |
| BDNF | NM_170731 | Homo | brain-derived neurotrophic factor |
| | | sapiens | |
| BDNF | NM_001143809 | Homo | brain-derived neurotrophic factor |
| | | sapiens | |
| BDNF | NM_001143807 | Homo | brain-derived neurotrophic factor |
| | | sapiens | |
| BDNF | NM_001143808 | Homo | brain-derived neurotrophic factor |
| | | sapiens | |
| BDNF | NM_007540 | Mus | brain derived neurotrophic factor |
| | | musculus | |
| BDNF | NM_001048141 | Mus | brain derived neurotrophic factor |
| | | musculus | |
| BDNF | NM_001048142 | Mus | brain derived neurotrophic factor |
| | | musculus | |
| BDNF | NM_001048139 | Mus | brain derived neurotrophic factor |
| | | musculus | |
| BRCA1 | NM_009764 | Mus | breast cancer 1 |
| | | musculus | |
| BRCA1 | NM_007296 | Homo | breast cancer 1, early onset |
| | | sapiens | |
| BRCA1 | NM_007300 | Homo | breast cancer 1, early onset |
| | | sapiens | |
| BRCA1 | NM_007297 | Homo | breast cancer 1, early onset |
| | | sapiens | |
| BRCA1 | NM_007303 | Homo | breast cancer 1, early onset |
| | | sapiens | |
| BRCA1 | NM_007298 | Homo | breast cancer 1, early onset |
| | | sapiens | |
| BRCA1 | NM_007302 | Homo | breast cancer 1, early onset |
| | | sapiens | |
| BRCA1 | NM_007299 | Homo | breast cancer 1, early onset |
| | | sapiens | |
| BRCA1 | NM_007304 | Homo | breast cancer 1, early onset |
| | | sapiens | |
| BRCA1 | NM_007294 | Homo | breast cancer 1, early onset |
| | | sapiens | |
| BRCA1 | NM_007305 | Homo | breast cancer 1, early onset |
| | | sapiens | |
| BRCA1 | NM_007295 | Homo | breast cancer 1, early onset |
| | | sapiens | |
| CD274 | NM_014143 | Homo | CD274 molecule |
| | | sapiens | |
| CD274 | NM_021893 | Mus | CD274 antigen |
| | | musculus | |
| CEP290 | NM_025114 | Homo | centrosomal protein 290 kDa |
| | | sapiens | |
| CEP290 | NM_146009 | Mus | centrosomal protein 290 |
| | | musculus | |
| CFTR | NM_000492 | Homo | cystic fibrosis transmembrane conductance |
| | | sapiens | regulator (ATP-binding cassette sub-family C, |
| | | | member 7) |
| CFTR | NM_021050 | Mus | cystic fibrosis transmembrane conductance |
| | | musculus | regulator homolog |
| EPO | NM_000799 | Homo | erythropoietin |
| | | sapiens | |
| EPO | NM_007942 | Mus | erythropoietin |
| | | musculus | |
| F7 | NM_000131 | Homo | coagulation factor VII (serum prothrombin |
| | | sapiens | conversion accelerator) |
| F7 | NM_019616 | Homo | coagulation factor VII (serum prothrombin |
| | | sapiens | conversion accelerator) |
| F7 | NM_010172 | Mus | coagulation factor VII |
| | | musculus | |
| F8 | NM_019863 | Homo | coagulation factor VIII, procoagulant |
| | | sapiens | component |
| F8 | NM_000132 | Homo | coagulation factor VIII, procoagulant |
| | | sapiens | component |
| F8 | NM_001161373 | Mus | coagulation factor VIII |
| | | musculus | |
| F8 | NM_001161374 | Mus | coagulation factor VIII |
| | | musculus | |
| F8 | NM_007977 | Mus | coagulation factor VIII |
| | | musculus | |
| FLI1 | NM_002017 | Homo | Friend leukemia virus integration 1 |
| | | sapiens | |
| FLI1 | NM_001167681 | Homo | Friend leukemia virus integration 1 |
| | | sapiens | |
| FLI1 | NM_008026 | Mus | Friend leukemia integration 1 |
| | | musculus | |
| FMR1 | NM_008031 | Mus | fragile X mental retardation syndrome 1 |
| | | musculus | homolog |
| FMR1 | NM_002024 | Homo | fragile X mental retardation 1 |
| | | sapiens | |
| FNDC5 | NM_001171941 | Homo | fibronectin type III domain containing 5 |
| | | sapiens | |
| FNDC5 | NM_153756 | Homo | fibronectin type III domain containing 5 |
| | | sapiens | |
| FNDC5 | NM_001171940 | Homo | fibronectin type III domain containing 5 |
| | | sapiens | |
| FNDC5 | NM_027402 | Mus | fibronectin type III domain containing 5 |
| | | musculus | |
| FOXP3 | NM_054039 | Mus | forkhead box P3 |
| | | musculus | |
| FOXP3 | NM_001114377 | Homo | forkhead box P3 |
| | | sapiens | |
| FOXP3 | NM_014009 | Homo | forkhead box P3 |
| | | sapiens | |
| FXN | NM_001161706 | Homo | frataxin |
| | | sapiens | |
| FXN | NM_181425 | Homo | frataxin |
| | | sapiens | |
| FXN | NM_000144 | Homo | frataxin |
| | | sapiens | |
| FXN | NM_008044 | Mus | frataxin |
| | | musculus | |
| GCH1 | NM_008102 | Mus | GTP cyclohydrolase 1 |
| | | musculus | |
| GCH1 | NM_000161 | Homo | GTP cyclohydrolase 1 |
| | | sapiens | |
| GCH1 | NM_001024070 | Homo | GTP cyclohydrolase 1 |
| | | sapiens | |
| GCH1 | NM_001024071 | Homo | GTP cyclohydrolase 1 |
| | | sapiens | |
| GCH1 | NM_001024024 | Homo | GTP cyclohydrolase 1 |
| | | sapiens | |
| GCK | NM_010292 | Mus | glucokinase |
| | | musculus | |
| GCK | NM_000162 | Homo | glucokinase (hexokinase 4) |
| | | sapiens | |
| GCK | NM_033508 | Homo | glucokinase (hexokinase 4) |
| | | sapiens | |
| GCK | NM_033507 | Homo | glucokinase (hexokinase 4) |
| | | sapiens | |
| GLP1R | NM_021332 | Mus | glucagon-like peptide 1 receptor; similar to |
| | | musculus | glucagon-like peptide-1 receptor |
| GLP1R | XM_001471951 | Mus | glucagon-like peptide 1 receptor; similar to |
| | | musculus | glucagon-like peptide-1 receptor |
| GLP1R | NM_002062 | Homo | glucagon-like peptide 1 receptor |
| | | sapiens | |
| GRN | NM_002087 | Homo | granulin |
| | | sapiens | |
| GRN | NM_008175 | Mus | granulin |
| | | musculus | |
| HAMP | NM_021175 | Homo | hepcidin antimicrobial peptide |
| | | sapiens | |
| HAMP | NM_032541 | Mus | hepcidin antimicrobial peptide |
| | | musculus | |
| HBA2 | NM_000517 | Homo | hemoglobin, alpha 2; hemoglobin, alpha 1 |
| | | sapiens | |
| HBA2 | NM_000558 | Homo | hemoglobin, alpha 2; hemoglobin, alpha 1 |
| | | sapiens | |
| HBB | NM_000518 | Homo | hemoglobin, beta |
| | | sapiens | |
| HBB | XM_921413 | Mus | hemoglobin beta chain complex |
| | | musculus | |
| HBB | XM_903245 | Mus | hemoglobin beta chain complex |
| | | musculus | |
| HBB | XM_921395 | Mus | hemoglobin beta chain complex |
| | | musculus | |
| HBB | XM_903244 | Mus | hemoglobin beta chain complex |
| | | musculus | |
| HBB | XM_903246 | Mus | hemoglobin beta chain complex |
| | | musculus | |
| HBB | XM_909723 | Mus | hemoglobin beta chain complex |
| | | musculus | |
| HBB | XM_921422 | Mus | hemoglobin beta chain complex |
| | | musculus | |
| HBB | XM_489729 | Mus | hemoglobin beta chain complex |
| | | musculus | |
| HBB | XM_903242 | Mus | hemoglobin beta chain complex |
| | | musculus | |
| HBB | XM_903243 | Mus | hemoglobin beta chain complex |
| | | musculus | |
| HBB | XM_921400 | Mus | hemoglobin beta chain complex |
| | | musculus | |
| HBD | NM_000519 | Homo | hemoglobin, delta |
| | | sapiens | |
| HBE1 | NM_005330 | Homo | hemoglobin, epsilon 1 |
| | | sapiens | |
| HBG1 | NM_000559 | Homo | hemoglobin, gamma A |
| | | sapiens | |
| HBG2 | NM_000184 | Homo | hemoglobin, gamma G |
| | | sapiens | |
| HPRT1 | NM_000194 | Homo | hypoxanthine phosphoribosyltransferase 1 |
| | | sapiens | |
| IDO1 | NM_008324 | Mus | indoleamine 2,3-dioxygenase 1 |
| | | musculus | |
| IDO1 | NM_002164 | Homo | indoleamine 2,3-dioxygenase 1 |
| | | sapiens | |
| IGF1 | NM_001111284 | Homo | insulin-like growth factor 1 (somatomedin C) |
| | | sapiens | |
| IGF1 | NM_001111285 | Homo | insulin-like growth factor 1 (somatomedin C) |
| | | sapiens | |
| IGF1 | NM_001111283 | Homo | insulin-like growth factor 1 (somatomedin C) |
| | | sapiens | |
| IGF1 | NM_000618 | Homo | insulin-like growth factor 1 (somatomedin C) |
| | | sapiens | |
| IGF1 | NM_001111274 | Mus | insulin-like growth factor 1 |
| | | musculus | |
| IGF1 | NM_010512 | Mus | insulin-like growth factor 1 |
| | | musculus | |
| IGF1 | NM_184052 | Mus | insulin-like growth factor 1 |
| | | musculus | |
| IGF1 | NM_001111276 | Mus | insulin-like growth factor 1 |
| | | musculus | |
| IGF1 | NM_001111275 | Mus | insulin-like growth factor 1 |
| | | musculus | |
| IL10 | NM_000572 | Mus | interleukin 10 |
| | | musculus | |
| IL10 | NM_010548 | Mus | interleukin 10 |
| | | musculus | |
| IL6 | NM_031168 | Mus | interleukin 6 |
| | | musculus | |
| IL6 | NM_000600 | Homo | interleukin 6 (interferon, beta 2) |
| | | sapiens | |
| KCNMA1 | NM_002247 | Homo | potassium large conductance calcium- |
| | | sapiens | activated channel, subfamily M, alpha |
| | | | member 1 |
| KCNMA1 | NM_001161352 | Homo | potassium large conductance calcium- |
| | | sapiens | activated channel, subfamily M, alpha |
| | | | member 1 |
| KCNMA1 | NM_001014797 | Homo | potassium large conductance calcium- |
| | | sapiens | activated channel, subfamily M, alpha |
| | | | member 1 |
| KCNMA1 | NM_001161353 | Homo | potassium large conductance calcium- |
| | | sapiens | activated channel, subfamily M, alpha |
| | | | member 1 |
| KCNMA1 | NM_010610 | Mus | potassium large conductance calcium- |
| | | musculus | activated channel, subfamily M, alpha |
| | | | member 1 |
| KCNMB1 | NM_031169 | Mus | potassium large conductance calcium- |
| | | musculus | activated channel, subfamily M, beta |
| | | | member 1 |
| KCNMB1 | NM_004137 | Homo | potassium large conductance calcium- |
| | | sapiens | activated channel, subfamily M, beta |
| | | | member 1 |
| KCNMB2 | NM_028231 | Mus | potassium large conductance calcium- |
| | | musculus | activated channel, subfamily M, beta |
| | | | member 2 |
| KCNMB2 | NM_005832 | Homo | potassium large conductance calcium- |
| | | sapiens | activated channel, subfamily M, beta |
| | | | member 2 |
| KCNMB2 | NM_181361 | Homo | potassium large conductance calcium- |
| | | sapiens | activated channel, subfamily M, beta |
| | | | member 2 |
| KCNMB3 | NM_171829 | Homo | potassium large conductance calcium- |
| | | sapiens | activated channel, subfamily M beta |
| | | | member 3 |
| KCNMB3 | NM_171828 | Homo | potassium large conductance calcium- |
| | | sapiens | activated channel, subfamily M beta |
| | | | member 3 |
| KCNMB3 | NM_001163677 | Homo | potassium large conductance calcium- |
| | | sapiens | activated channel, subfamily M beta |
| | | | member 3 |
| KCNMB3 | NM_014407 | Homo | potassium large conductance calcium- |
| | | sapiens | activated channel, subfamily M beta |
| | | | member 3 |
| KCNMB3 | NM_171830 | Homo | potassium large conductance calcium- |
| | | sapiens | activated channel, subfamily M beta |
| | | | member 3 |
| KCNMB3 | XM_001475546 | Mus | potassium large conductance calcium- |
| | | musculus | activated channel, subfamily M, beta |
| | | | member 3 |
| KCNMB3 | XM_912348 | Mus | potassium large conductance calcium- |
| | | musculus | activated channel, subfamily M, beta |
| | | | member 3 |
| KCNMB4 | NM_021452 | Mus | potassium large conductance calcium- |
| | | musculus | activated channel, subfamily M, beta |
| | | | member 4 |
| KCNMB4 | NM_014505 | Homo | potassium large conductance calcium- |
| | | sapiens | activated channel, subfamily M, beta |
| | | | member 4 |
| KLF1 | NM_010635 | Mus | Kruppel-like factor 1 (erythroid) |
| | | musculus | |
| KLF1 | NM_006563 | Homo | Kruppel-like factor 1 (erythroid) |
| | | sapiens | |
| KLF4 | NM_010637 | Mus | Kruppel-Iike factor 4 (gut) |
| | | musculus | |
| KLF4 | NM_004235 | Homo | Kruppel-like factor 4 (gut) |
| | | sapiens | |
| LAMA1 | NM_005559.3 | Homo | laminin, alpha 1 |
| | | sapiens | |
| LAMA1 | NM_008480.2 | Mus | laminin, alpha 1 |
| | | musculus | |
| LDLR | NM_000527 | Homo | low density lipoprotein receptor |
| | | sapiens | |
| LDLR | NM_010700 | Mus | low density lipoprotein receptor |
| | | musculus | |
| MBNL1 | NM_021038.3, | Homo | muscleblind-like splicing regulator 1 |
| | NM_020007.3, | sapiens | |
| | NM_207293.1, | | |
| | NM_207294.1, | | |
| | NM_207295.1, | | |
| | NM_207296.1, | | |
| | NM_207297.1 | | |
| MBNL1 | NM_001253708.1, | Mus | muscleblind-like 1 (Drosophila) |
| | NM_001253709.1, | musculus | |
| | NM_001253710.1, | | |
| | NM_001253711.1, | | |
| | NM_001253713.1, | | |
| | NM_020007.3 | | |
| MECP2 | NM_010788 | Mus | methyl CpG binding protein 2 |
| | | musculus | |
| MECP2 | NM_001081979 | Mus | methyl CpG binding protein 2 |
| | | musculus | |
| MECP2 | NM_001110792 | Homo | methyl CpG binding protein 2 |
| | | sapiens | (Rett syndrome) |
| MECP2 | NM_004992 | Homo | methyl CpG binding protein 2 |
| | | sapiens | (Rett syndrome) |
| MERTK | NM_006343.2 | Homo | MER proto-oncogene, tyrosine kinase |
| | | sapiens | |
| MERTK | NM_008587.1 | Mus | c-mer proto-oncogene tyrosine kinase |
| | | musculus | |
| MSX2 | NM_013601 | Mus | similar to homeobox protein; homeobox, |
| | | musculus | msh-like 2 |
| MSX2 | XM_001475886 | Mus | similar to homeobox protein; homeobox, |
| | | musculus | msh-like 2 |
| MSX2 | NM_002449 | Homo | msh homeobox 2 |
| | | sapiens | |
| MYBPC3 | NM_008653 | Mus | myosin binding protein C, cardiac |
| | | musculus | |
| MYBPC3 | NM_000256 | Homo | myosin binding protein C, cardiac |
| | | sapiens | |
| NANOG | NM_024865 | Homo | Nanog homeobox pseudogene 8; Nanog |
| | | sapiens | homeobox |
| NANOG | XM_001471588 | Mus | similar to Nanog homeobox; Nanog |
| | | musculus | homeobox |
| NANOG | NM_028016 | Mus | similar to Nanog homeobox; Nanog |
| | | musculus | homeobox |
| NANOG | NM_001080945 | Mus | similar to Nanog homeobox; Nanog |
| | | musculus | homeobox |
| NF1 | NM_000267 | Homo | neurofibromin 1 |
| | | sapiens | |
| NF1 | NM_001042492 | Homo | neurofibromin 1 |
| | | sapiens | |
| NF1 | NM_001128147 | Homo | neurofibromin 1 |
| | | sapiens | |
| NF1 | NM_010897 | Mus | neurofibromatosis 1 |
| | | musculus | |
| NKX2-1 | NM_001079668 | Homo | NK2 homeobox 1 |
| | | sapiens | |
| NKX2-1 | NM_003317 | Homo | NK2 homeobox 1 |
| | | sapiens | |
| NKX2-1 | XM_002344771 | Homo | KK2 homeobox 1 |
| | | sapiens | |
| NKX2-1 | NM_009385 | Mus | KK2 homeobox 1 |
| | | musculus | |
| NKX2-1 | NM_001146198 | Mus | NK2 homeobox 1 |
| | | musculus | |
| PAH | NM_008777 | Mus | phenylalanine hydroxylase |
| | | musculus | |
| PAH | NM_000277 | Homo | phenylalanine hydroxylase |
| | | sapiens | |
| PTEN | NM_000314 | Homo | phosphatase and tensin homolog; |
| | | sapiens | phosphatase and tensin homolog |
| | | | pseudogene 1 |
| PTEN | NM_177096 | Mus | phosphatase and tensin homolog |
| | | musculus | |
| PTEN | NM_008960 | Mus | phosphatase and tensin homolog |
| | | musculus | |
| PTGS2 | NM_011198 | Mus | prostaglandin-endoperoxide synthase 2 |
| | | musculus | |
| PTGS2 | NM_000963 | Homo | prostaglandin-endoperoxide synthase 2 |
| | | sapiens | (prostaglandin G/H synthase and |
| | | | cyclooxygenase) |
| RB1 | NM_009029 | Mus | retinoblastoma 1 |
| | | musculus | |
| RB1 | NM_000321 | Homo | retinoblastoma 1 |
| | | sapiens | |
| RPS14 | NM_020600 | Mus | predicted gene 6204; ribosomal protein S14 |
| | | musculus | |
| RPS14 | NM_001025071 | Homo | ribosomal protein S14 |
| | | sapiens | |
| RPS14 | NM_005617 | Homo | ribosomal protein S14 |
| | | sapiens | |
| RPS14 | NM_001025070 | Homo | ribosomal protein S14 |
| | | sapiens | |
| RPS19 | XM_204069 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein S19; |
| | | | predicted gene 9091; predicted gene 6636; |
| | | | predicted gene 14072 |
| RPS19 | XM_991053 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein S19; |
| | | | predicted gene 9091; predicted gene 6636; |
| | | | predicted gene 14072 |
| RPS19 | XM_905004 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein S19; |
| | | | predicted gene 9091; predicted gene 6636; |
| | | | predicted gene 14072 |
| RPS19 | XM_001005575 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein S19; |
| | | | predicted gene 9091; predicted gene 6636; |
| | | | predicted gene 14072 |
| RPS19 | NM_023133 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein S19; |
| | | | predicted gene 9091; predicted gene 6636; |
| | | | predicted gene 14072 |
| RPS19 | XM_994263 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein S19; |
| | | | predicted gene 9091; predicted gene 6636; |
| | | | predicted gene 14072 |
| RPS19 | XM_001481027 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein S19; |
| | | | predicted gene 9091; predicted gene 6636; |
| | | | predicted gene 14072 |
| RPS19 | XM_913504 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein S19; |
| | | | predicted gene 9091; predicted gene 6636; |
| | | | predicted gene 14072 |
| RPS19 | XM_001479631 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein S19; |
| | | | predicted gene 9091; predicted gene 6636; |
| | | | predicted gene 14072 |
| RPS19 | XM_902221 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein S19; |
| | | | predicted gene 9091; predicted gene 6636; |
| | | | predicted gene 14072 |
| RPS19 | XM_893968 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein 519; |
| | | | predicted gene 9091; predicted gene 6636; |
| | | | predicted gene 14072 |
| RPS19 | NM_001022 | Homo | ribosomal protein S19 pseudogene 3; |
| | | sapiens | ribosomal protein S19 |
| SCARB1 | NM_016741 | Mus | scavenger receptor class B, member 1 |
| | | musculus | |
| SCARB1 | NM_001082959 | Homo | scavenger receptor class B, member 1 |
| | | sapiens | |
| SCARB1 | NM_005505 | Homo | scavenger receptor class B, member 1 |
| | | sapiens | |
| SERPINF1 | NM_011340 | Mus | serine (or cysteine) peptidase inhibitor, |
| | | musculus | clade F, member 1 |
| SERPINF1 | NM_002615 | Homo | serpin peptidase inhibitor, clade F (alpha-2 |
| | | sapiens | antiplasmin, pigment epithelium derived |
| | | | factor), member 1 |
| SIRT1 | NM_001159590 | Mus | sirtuin 1 (silent mating type information |
| | | musculus | regulation 2, homolog) 1 (S. cerevisiae) |
| SIRT1 | NM_019812 | Mus | sirtuin 1 (silent mating type information |
| | | musculus | regulation 2, homolog) 1 (S. cerevisiae) |
| SIRT1 | NM_001159589 | Mus | sirtuin 1 (silent mating type information |
| | | musculus | regulation 2, homolog) 1 (S. cerevisiae) |
| SIRT1 | NM_012238 | Homo | sirtuin (silent mating type information |
| | | sapiens | regulation 2 homolog) 1 (S. cerevisiae) |
| SIRT1 | NM_001142498 | Homo | sirtuin (silent mating type information |
| | | sapiens | regulation 2 homolog) 1 (S. cerevisiae) |
| SIRT6 | NM_016539 | Homo | sirtuin (silent mating type information |
| | | sapiens | regulation 2 homolog) 6 (S. cerevisiae) |
| SIRT6 | NM_001163430 | Mus | sirtuin 6 (silent mating type information |
| | | musculus | regulation 2, homolog) 6 (S. cerevisiae) |
| SIRT6 | NM_181586 | Mus | sirtuin 6 (silent mating type information |
| | | musculus | regulation 2, homolog) 6 (S. cerevisiae) |
| SMAD7 | NM_005904 | Homo | SMAD family member 7 |
| | | sapiens | |
| SMAD7 | NM_001042660 | Mus | MAD homolog 7 (Drosophila) |
| | | musculus | |
| SMN1 | NM_000344.3 | Homo | Survival Motor Neuron 1 |
| | | sapiens | |
| SMN1 | NM_022874.2 | Homo | Survival Motor Neuron 1 |
| | | sapiens | |
| SMN2 | NM_017411.3 | Homo | Survival Motor Neuron 2 |
| | NM_022875.2 | sapiens | |
| | NM_022876.2 | | |
| | NM_022877.2 | | |
| SSPN | NM_001135823.1, | Homo | sarcospan |
| | NM_005086.4 | sapiens | |
| SSPN | NM_010656.2 | Homo | sarcospan |
| | | sapiens | |
| ST7 | NM_021908 | Homo | suppression of tumorigenicity 7 |
| | | sapiens | |
| ST7 | NM_018412 | Homo | suppression of tumorigenicity 7 |
| | | sapiens | |
| STAT3 | NM_213660 | Mus | similar to Stat3B; signal transducer and |
| | | musculus | activator of transcription 3 |
| STAT3 | XM_001474017 | Mus | similar to Stat3B; signal transducer and |
| | | musculus | activator of transcription 3 |
| STAT3 | NM_213659 | Mus | similar to Stat3B; signal transducer and |
| | | musculus | activator of transcription 3 |
| STAT3 | NM_011486 | Mus | similar to Stat3B; signal transducer and |
| | | musculus | activator of transcription 3 |
| STAT3 | NM_213662 | Homo | signal transducer and activator of |
| | | sapiens | transcription 3 (acute-phase response factor) |
| STAT3 | NM_003150 | Homo | signal transducer and activator of |
| | | sapiens | transcription 3 (acute-phase response factor) |
| STAT3 | NM_139276 | Homo | signal transducer and activator of |
| | | sapiens | transcription 3 (acute-phase response factor) |
| UTRN | NM_007124 | Homo | utrophin |
| | | sapiens | |
| UTRN | NM_011682 | Mus | utrophin |
| | | musculus | |
| NFE2L2 | NM_001145412.2, | Homo | nuclear factor, erythroid 2-like 2 |
| | NM_001145413.2, | sapiens | |
| | NM_006164.4 | | |
| NFE2L2 | NM_010902.3 | Mus | nuclear factor, erythroid 2-like 2 |
| | | musculus | |
| ACTB | NM_001101.3 | Homo | actin, beta |
| | | sapiens | |
| ACTB | NM_007393.3 | Mus | actin, beta |
| | | musculus | |
| ANRIL | NR_003529.3, | Homo | CDKN2B antisense RNA 1 |
| | NR_047532.1, | sapiens | (also called CDKN2B) |
| | NR_047533.1, | | |
| | NR_047534.1, | | |
| | NR_047535.1, | | |
| | NR_047536.1, | | |
| | NR_047538.1, | | |
| | NR_047539.1, | | |
| | NR_047540.1, | | |
| | NR_047541.1, | | |
| | NR_047542.1, | | |
| | NR 047543.1 | | |
| HOTAIR | NR_003716.3, | Homo | HOX transcript antisense RNA |
| | NR_047517.1, | sapiens | |
| | NR_047518.1 | | |
| HOT AIR | NR_047528.1 | Mus | HOX transcript antisense RNA |
| | | musculus | |
| DINO | JX993265 | Homo | Damage Induced NOncoding |
| | | sapiens | |
| DINO | JX993266 | Mus | Damage Induced NOncoding |
| | | musculus | |
| HOTTIP | NR_037843.3 | Homo | HOXA distal transcript antisense RNA |
| | | sapiens | |
| HOTTIP | NR_110441.1, | Mus | Hoxa distal transcript antisense RNA |
| | NR 110442.1 | musculus | |
| NEST | NR_104124.1 | Homo | Homo sapiens IFNG antisense RNA 1 |
| | | sapiens | (IFNG-AS1), transcript variant 1, long |
| | | | non-coding RNA. |
| NEST | NR_104123.1 | Mus | Theiler's murine encephalomyelitis virus |
| | | musculus | persistence candidate gene 1 |
| THRB | NM_000461.4 | Homo | thyroid hormone receptor, beta |
| | | sapiens | |
| THRB | NM_001128176.2 | Homo | thyroid hormone receptor, beta |
| | | sapiens | |
| THRB | NM_001128177.1 | Homo | thyroid hormone receptor, beta |
| | | sapiens | |
| THRB | NM_001252634.1 | Homo | thyroid hormone receptor, beta |
| | | sapiens | |
| THRB | NM_00H13417.1 | Mus | thyroid hormone receptor, beta |
| | | musculus | |
| THRB | NM_009380.3 | Mus | thyroid hormone receptor, beta |
| | | musculus | |
| NR1H4 | NM_001206977.1 | Homo | nuclear receptor subfamily 1, group H, |
| | | sapiens | member 4 |
| NR1H4 | NM_001206978.1 | Homo | nuclear receptor subfamily 1, group H, |
| | | sapiens | member 4 |
| NR1H4 | NM_001206979.1 | Homo | nuclear receptor subfamily 1, group H, |
| | | sapiens | member 4 |
| NR1H4 | NM_001206992.1 | Homo | nuclear receptor subfamily 1, group H, |
| | | sapiens | member 4 |
| NR1H4 | NM_001206993.1 | Homo | nuclear receptor subfamily 1, group H, |
| | | sapiens | member 4 |
| NR1H4 | NM_005123.3 | Homo | nuclear receptor subfamily 1, group H, |
| | | sapiens | member 4 |
| NR1H4 | NM_001163504.1 | Mus | nuclear receptor subfamily 1, group H, |
| | | musculus | member 4 |
| NR1H4 | NM_001163700.1 | Mus | nuclear receptor subfamily 1, group H, |
| | | musculus | member 4 |
| NR1H4 | NM_009108.2 | Mus | nuclear receptor subfamily 1, group H, |
| | | musculus | member 4 |
| PRKAA1 | NM_006251.5 | Homo | protein kinase, AMP-activated, alpha 1 |
| | | sapiens | catalytic subunit |
| PRKAA1 | NM_206907.3 | Homo | protein kinase, AMP-activated, alpha 1 |
| | | sapiens | catalytic subunit |
| PRKAA1 | NM_001013367.3 | Mus | protein kinase, AMP-activated, alpha 1 |
| | | musculus | catalytic subunit |
| PRKAA2 | NM_006252.3 | Homo | protein kinase, AMP-activated, alpha 2 |
| | | sapiens | catalytic subunit |
| PRKAA2 | NM_178143.2 | Mus | protein kinase, AMP-activated, alpha 2 |
| | | musculus | catalytic subunit |
| PRKAB1 | NM_006253.4 | Homo | protein kinase, AMP-activated, beta 1 |
| | | sapiens | non-catalytic subunit |
| PRKAB1 | NM_031869.2 | Mus | protein kinase, AMP-activated, beta 1 |
| | | musculus | non-catalytic subunit |
| PRKAB2 | NM_005399.4 | Homo | protein kinase, AMP-activated, beta 2 |
| | | sapiens | non-catalytic subunit |
| PRKAB2 | NM_182997.2 | Mus | protein kinase, AMP-activated, beta 2 |
| | | musculus | non-catalytic subunit |
| PRKAG1 | NM_001206709.1 | Homo | protein kinase, AMP-activated, gamma 1 |
| | | sapiens | non-catalytic subunit |
| PRKAG1 | NM_001206710.1 | Homo | protein kinase, AMP-activated, gamma 1 |
| | | sapiens | non-catalytic subunit |
| PRKAG1 | NM_002733.4 | Homo | protein kinase, AMP-activated, gamma 1 |
| | | sapiens | non-catalytic subunit |
| PRKAG1 | NM_016781.2 | Mus | protein kinase, AMP-activated, gamma 1 |
| | | musculus | non-catalytic subunit |
| PRKAG2 | NM_001040633.1 | Homo | protein kinase, AMP-activated, gamma 2 |
| | | sapiens | non-catalytic subunit |
| PRKAG2 | NM_001304527.1 | Homo | protein kinase, AMP-activated, gamma 2 |
| | | sapiens | non-catalytic subunit |
| PRKAG2 | NM_001304531.1 | Homo | protein kinase, AMP-activated, gamma 2 |
| | | sapiens | non-catalytic subunit |
| PRKAG2 | NM_016203.3 | Homo | protein kinase, AMP-activated, gamma 2 |
| | | sapiens | non-catalytic subunit |
| PRKAG2 | NM_024429.1 | Homo | protein kinase, AMP-activated, gamma 2 |
| | | sapiens | non-catalytic subunit |
| PRKAG2 | NM_001170555.1 | Mus | protein kinase, AMP-activated, gamma 2 |
| | | musculus | non-catalytic subunit |
| PRKAG2 | NM_001170556.1 | Mus | protein kinase, AMP-activated, gamma 2 |
| | | musculus | non-catalytic subunit |
| PRKAG2 | NM_145401.2 | Mus | protein kinase, AMP-activated, gamma 2 |
| | | musculus | non-catalytic subunit |
| PRKAG3 | NM_017431.2 | Homo | protein kinase, AMP-activated, gamma 3 |
| | | sapiens | non-catalytic subunit |
| PRKAG3 | NM_153744.3 | Mus | protein kinase, AMP-activated, gamma 3 |
| | | musculus | non-catalytic subunit |

Methods of modulating (e.g., upregulating or downregulating) gene expression are provided, in some embodiments, that may be carried out in vitro, ex vivo, or in vivo. It is understood that any reference to uses of compounds throughout the description contemplates use of the compound in preparation of a pharmaceutical composition or medicament for use in the treatment of a condition associated with increased or decreased levels or activity of a target gene. Thus, as one nonlimiting example, this aspect of the invention includes use of such single stranded oligonucleotides in the preparation of a medicament for use in the treatment of disease, wherein the treatment involves upregulating or downregulating expression of a target gene.

In further aspects of the invention, methods are provided for selecting a candidate oligonucleotide for modulating (e.g., upregulating or downregulating) expression of a target gene. The methods generally involve selecting as a candidate oligonucleotide, a single stranded oligonucleotide comprising a nucleotide sequence that is complementary to a lancRNA or to a chromosomal region that encodes a lancRNA, e.g., a region within 5 kb of a transcriptional boundary of a target gene. In some embodiments, sets of oligonucleotides may be selected that are enriched (e.g., compared with a random selection of oligonucleotides) in oligonucleotides that modulate (e.g., upregulate or downregulate) expression of a target gene.

### Single Stranded Oligonucleotides for Modulating Expression of a Target Gene

In one aspect of the invention, single stranded oligonucleotides complementary to a lancRNA or to a chromosomal region that encodes a lancRNA, e.g., a region within 5 kb of a transcriptional boundary of a target gene, are provided for modulating expression of the target gene in a cell. In some embodiments, expression of the target gene is upregulated or increased. The oligonucleotide may be selected using any of the methods disclosed herein for selecting a candidate oligonucleotide for modulating expression of a target gene.

The single stranded oligonucleotide may comprise a region of complementarity that is complementary with a lancRNA or with a chromosomal region that encodes a lancRNA, e.g., a region within 5 kb of a transcriptional boundary of a target gene. The region of complementarity of the single stranded oligonucleotide may be complementary with at least 5, e.g., at least 6, at least 7, at least 8, at least 9, at least 10, at least 15 or more consecutive nucleotides of the lancRNA or chromosomal region that encodes the lancRNA, e.g., a region within 5 kb of a transcriptional boundary of a target gene.

The chromosomal region encoding the lancRNA may map to a position in a chromosome between 10 kilobases (e.g., 5 kb, 4, kb, 2 kb, 1 kb, 500 bp, 400 bp, 300 bp, 200 bp, 100 bp) upstream and 10 kilobases (e.g., 5 kb, 4, kb, 2 kb, 1 kb, 500 bp, 400 bp, 300 bp, 200 bp, 100 bp) downstream of a transcriptional start site of the target gene or 10 kilobases (e.g., 5 kb, 4, kb, 2 kb, 1 kb, 500 bp, 400 bp, 300 bp, 200 bp, 100 bp) upstream and 10 kilobases (e.g., 5 kb, 4, kb, 2 kb, 1 kb, 500 bp, 400 bp, 300 bp, 200 bp, 100 bp) downstream of a transcriptional end site of the target gene.

The single stranded oligonucleotide may have a sequence that does not contain guanosine nucleotide stretches (e.g., 3 or more, 4 or more, 5 or more, 6 or more consecutive guanosine nucleotides). In some embodiments, oligonucleotides having guanosine nucleotide stretches have increased non-specific binding and/or off-target effects, compared with oligonucleotides that do not have guanosine nucleotide stretches.

The single stranded oligonucleotide may have a sequence that has less than a threshold level of sequence identity with every sequence of nucleotides, of equivalent length, that map to a genomic position encompassing or in proximity to an off-target gene. For example, an oligonucleotide may be designed to ensure that it does not have a sequence that maps to genomic positions encompassing or in proximity with all known genes (e.g., all known protein coding genes) other than the target gene. In a similar embodiment, an oligonucleotide may be designed to ensure that it does not have a sequence that maps to any other known lancRNAs. In either case, the oligonucleotide is expected to have a reduced likelihood of having off-target effects. The threshold level of sequence identity may be 50%, 60%, 70%, 80%, 85%, 90%, 95%, 99% or 100% sequence identity.

The single stranded oligonucleotide may have a sequence that is has greater than 30% G-C content, greater than 40% G-C content, greater than 50% G-C content, greater than 60% G-C content, greater than 70% G-C content, or greater than 80% G-C content. The single stranded oligonucleotide may have a sequence that has up to 100% G-C content, up to 95% G-C content, up to 90% G-C content, or up to 80% G-C content. In some embodiments in which the oligonucleotide is 8 to 10 nucleotides in length, all but 1, 2, 3, 4, or 5 of the nucleotides of the complementary sequence of the lancRNA are cytosine or guanosine nucleotides. In some embodiments, the sequence of the lancRNA to which the single stranded oligonucleotide is complementary comprises no more than 3 nucleotides selected from adenine and uracil.

The single stranded oligonucleotide may be complementary to a chromosome of a different species (e.g., a mouse, rat, rabbit, goat, monkey, etc.) at a position that encompasses or that is in proximity to that species' homolog of a target gene. The single stranded oligonucleotide may be complementary to a human genomic region encompassing or in proximity to the target gene and also be complementary to a mouse genomic region encompassing or in proximity to the mouse homolog of the target gene. Oligonucleotides having these characteristics may be tested in vivo or in vitro for efficacy in multiple species (e.g., human and mouse). This approach also facilitates development of clinical candidates for treating human disease by selecting a species in which an appropriate animal exists for the disease.

According to some aspects, single stranded oligonucleotides are provided that have a region of complementarity that is complementarty with (e.g., at least 5 consecutive nucleotides of) a lancRNA of a target gene. In some embodiments, the oligonucleotide has at least one of the following features: a) a sequence that is 5′X-Y-Z, in which X is any nucleotide and in which X is at the 5′ end of the oligonucleotide, Y is a nucleotide sequence of 6 nucleotides in length that is not a human seed sequence of a microRNA, and Z is a nucleotide sequence of 1 to 23 nucleotides in length; b) a sequence that does not comprise three or more consecutive guanosine nucleotides; c) a sequence that has less than a threshold level of sequence identity with every sequence of nucleotides, of equivalent length to the second nucleotide sequence, that are between 50 kilobases upstream of a 5′-end of an off-target gene and 50 kilobases downstream of a 3′-end of the off-target gene; and d) a sequence that has greater than 60% G-C content. In some embodiments, the single stranded oligonucleotide has at least two of features a), b), c), and d), each independently selected. In some embodiments, the single stranded oligonucleotide has at least three of features a), b), c), and d), each independently selected. In some embodiments, the single stranded oligonucleotide has at least four of features a), b), c), and d), each independently selected. In some embodiments, the single stranded oligonucleotide has each of features a), b), c), and d). In certain embodiments, the oligonucleotide has the sequence 5′X-Y-Z, in which the oligonucleotide is 8-50 nucleotides in length.

In some embodiments, the region of complementarity of the single stranded oligonucleotide is complementary with 5 to 15, 6 to 15, 7 to 15, 8 to 15, 5 to 30, 6 to 30, 7 to 30, 8 to 30, 8 to 40, or 10 to 50, or 5 to 50, or 5 to 40 bases, e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 consecutive nucleotides of a lancRNA. In some embodiments, the region of complementarity is complementary with at least 8 consecutive nucleotides of a lancRNA.

Complementary, as the term is used in the art, refers to the capacity for precise pairing between two nucleotides. For example, if a nucleotide at a certain position of an oligonucleotide is capable of hydrogen bonding with a nucleotide at the same position of lancRNA, then the single stranded nucleotide and lancRNA are considered to be complementary to each other at that position. The single stranded nucleotide and lancRNA are complementary to each other when a sufficient number of corresponding positions in each molecule are occupied by nucleotides that can hydrogen bond with each other through their bases. Thus, “complementary” is a term which is used to indicate a sufficient degree of complementarity or precise pairing such that stable and specific binding occurs between the single stranded nucleotide and lancRNA. For example, if a base at one position of a single stranded nucleotide is capable of hydrogen bonding with a base at the corresponding position of a lancRNA, then the bases are considered to be complementary to each other at that position. 100% complementarity is not required.

The single stranded oligonucleotide may be at least 80% complementary to (optionally one of at least 85%, 90%, 91%, 92%, 93%, 94%, 95%, 96%, 97%, 98%, 99% or 100% complementary to) the consecutive nucleotides of a lancRNA. In some embodiments the single stranded oligonucleotide may contain 1, 2 or 3 base mismatches compared to the portion of the consecutive nucleotides of a lancRNA. In some embodiments the single stranded oligonucleotide may have up to 3 mismatches over 15 bases, or up to 2 mismatches over 10 bases.

It is understood in the art that a complementary nucleotide sequence need not be 100% complementary to that of its target to be specifically hybridizable. In some embodiments, a complementary nucleic acid sequence for purposes of the present disclosure is specifically hybridizable when binding of the sequence to the target molecule (e.g., lancRNA) interferes with the normal function of the target (e.g., lancRNA) to cause a loss of activity and there is a sufficient degree of complementarity to avoid non-specific binding of the sequence to non-target sequences under conditions in which avoidance of non-specific binding is desired, e.g., under physiological conditions in the case of in vivo assays or therapeutic treatment, and in the case of in vitro assays, under conditions in which the assays are performed under suitable conditions of stringency.

In some embodiments, the single stranded oligonucleotide is 7, 8, 9, 10, 11, 12, 13, 14, 15, 16, 17, 18, 19, 20, 21, 22, 23, 24, 25, 26, 27, 28, 29, 30, 35, 40, 45, 50 or more nucleotides in length. In some embodiments, the oligonucleotide is 8 to 30 nucleotides in length.

In some embodiments, the chromosomal region encoding the lancRNA occurs on the same DNA strand as a gene sequence (sense). In some embodiments, the chromosomal region encoding the lancRNA occurs on the opposite DNA strand as a gene sequence (anti-sense). Oligonucleotides complementary to a lancRNA or the chromosomal region encoding the lancRNA can bind either sense or anti-sense sequences. Base pairings may include both canonical Watson-Crick base pairing and non-Watson-Crick base pairing (e.g., Wobble base pairing and Hoogsteen base pairing). It is understood that for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

In some embodiments, any one or more thymidine (T) nucleotides (or modified nucleotide thereof) or uridine (U) nucleotides (or a modified nucleotide thereof) in a sequence provided herein, including a sequence provided in the sequence listing, may be replaced with any other nucleotide suitable for base pairing (e.g., via a Watson-Crick base pair) with an adenosine nucleotide. In some embodiments, any one or more thymidine (T) nucleotides (or modified nucleotide thereof) or uridine (U) nucleotides (or a modified nucleotide thereof) in a sequence provided herein, including a sequence provided in the sequence listing, may be suitably replaced with a different pyrimidine nucleotide or vice versa. In some embodiments, any one or more thymidine (T) nucleotides (or modified nucleotide thereof) in a sequence provided herein, including a sequence provided in the sequence listing, may be suitably replaced with a uridine (U) nucleotide (or a modified nucleotide thereof) or vice versa.

In some embodiments, GC content of the single stranded oligonucleotide is preferably between about 30-60%. Contiguous runs of three or more Gs or Cs may not be preferable in some embodiments. Accordingly, in some embodiments, the oligonucleotide does not comprise a stretch of three or more guanosine nucleotides.

In some embodiments, it has been found that single stranded oligonucleotides disclosed herein may increase expression of mRNA corresponding to the gene by at least about 50% (i.e. 150% of normal or 1.5 fold), or by about 2 fold to about 5 fold. In some embodiments, expression may be increased by at least about 15 fold, 20 fold, 30 fold, 40 fold, 50 fold or 100 fold, or any range between any of the foregoing numbers. It has also been found that increased mRNA expression has been shown to correlate to increased protein expression.

In some or any of the embodiments of oligonucleotides described herein, or processes for designing or synthesizing them, the oligonucleotides will upregulate gene expression and may specifically bind or specifically hybridize or be complementary to a lancRNA that is transcribed from the same strand (the sense strand) of a protein coding reference gene. In some or any of the embodiments of oligonucleotides described herein, or processes for designing or synthesizing them, the oligonucleotides will upregulate gene expression and may specifically bind or specifically hybridize or be complementary to a lancRNA that is transcribed from the opposite strand (the antisense strand) of a protein coding reference gene. The oligonucleotide may bind to a region of the lancRNA that is transcribed from a region within or overlaps with an 5′ UTR, 3′ UTR, a translation initiation region, or a translation termination region of a target gene. The oligonucleotide may bind to a region of the lancRNA that is transcribed from a region upstream of an 5′ UTR or a translation initiation region or from a region downstream of a 3′ UTR or a translation termination region of a target gene.

The oligonucleotides described herein may be modified, e.g., comprise a modified sugar moiety, a modified internucleoside linkage, a modified nucleotide and/or combinations thereof.

Any of the oligonucleotides disclosed herein may be linked to one or more other oligonucleotides disclosed herein by a linker, e.g., a cleavable linker.

### Method for Selecting Candidate Oligonucleotides for Modulating Expression of a Target Gene

Methods are provided herein for selecting a candidate oligonucleotide for modulating (e.g., activating or enhancing) expression of a target gene. The target selection methods may generally involve steps for selecting single stranded oligonucleotides having any of the structural and functional characteristics disclosed herein. Typically, the methods involve one or more steps aimed at identifying oligonucleotides that target a lancRNA that is functionally related to a target gene, for example a lancRNA that regulates expression of a target gene (e.g., in a cis-regulatory manner). In some embodiments, “cis-regulatory manner” means that the lancRNA regulates expression of genes in the locus from which the lancRNA is expressed.

Methods of selecting a candidate oligonucleotide may involve selecting a region that encodes a lancRNA that maps to a chromosomal position encompassing or in proximity to a transcriptional boundary of the target gene. The region encoding the lancRNA may map to the strand of the chromosome comprising the sense strand of the target gene, in which case the candidate oligonucleotide is complementary to the sense strand of the target gene (i.e., the oligonucleotide is antisense to the target gene). Alternatively, the region encoding the lancRNA may map to the strand of the chromosome comprising the antisense strand of the target gene, in which case the oligonucleotide is complementary to the antisense strand (the template strand) of the target gene (i.e., the oligonucleotide is sense to the target gene).

Methods for selecting a set of candidate oligonucleotides that is enriched in oligonucleotides that modulate (e.g., activate) expression of a target gene may involve selecting one or more regions that encode lancRNAs that map to a chromosomal position that encompasses or that is in proximity to a transcriptional boundary of the target gene and selecting a set of oligonucleotides, in which each oligonucleotide in the set comprises a nucleotide sequence that is complementary with the one or more regions. As used herein, the phrase, “a set of oligonucleotides that is enriched in oligonucleotides that modulate (e.g., activate) expression of” refers to a set of oligonucleotides that has a greater number of oligonucleotides that modulate (e.g., activate) expression of a target gene compared with a random selection of oligonucleotides of the same physicochemical properties (e.g., the same GC content, Tₘ, length etc.) as the enriched set.

Where the design and/or synthesis of a single stranded oligonucleotide involves design and/or synthesis of a sequence that is complementary to a nucleic acid or lancRNA described by such sequence information, the skilled person is readily able to determine the complementary sequence, e.g., through understanding of Watson Crick base pairing rules which form part of the common general knowledge in the field.

In some embodiments design and/or synthesis of a single stranded oligonucleotide involves manufacture of an oligonucleotide from starting materials by techniques known to those of skill in the art, where the synthesis may be based on a sequence of a lancRNA, a region encoding a lancRNA, or portion thereof.

Methods of design and/or synthesis of a single stranded oligonucleotide may involve one or more of the steps of:

Identifying and/or selecting a chromosomal region within 5 kb of a transcriptional boundary;

Designing a nucleic acid sequence having a desired degree of sequence identity or complementarity to the region or a portion thereof;

Synthesizing a single stranded oligonucleotide to the designed sequence;

Purifying the synthesized single stranded oligonucleotide; and

Optionally mixing the synthesized single stranded oligonucleotide with at least one pharmaceutically acceptable diluent, carrier or excipient to form a pharmaceutical composition or medicament.

Single stranded oligonucleotides so designed and/or synthesized may be useful in method of modulating gene expression as described herein.

Preferably, single stranded oligonucleotides of the invention are synthesized chemically. Oligonucleotides used to practice this invention can be synthesized in vitro by well-known chemical synthesis techniques.

Oligonucleotides of the invention can be stabilized against nucleolytic degradation such as by the incorporation of a modification, e.g., a nucleotide modification. For example, nucleic acid sequences of the invention include a phosphorothioate at least the first, second, or third internucleotide linkage at the 5′ or 3′ end of the nucleotide sequence. As another example, the nucleic acid sequence can include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). As another example, the nucleic acid sequence can include at least one 2′-O-methyl-modified nucleotide, and in some embodiments, all of the nucleotides include a 2′-O-methyl modification. In some embodiments, the nucleic acids are “locked,” i.e., comprise nucleic acid analogues in which the ribose ring is “locked” by a methylene bridge connecting the 2′-O atom and the 4′-C atom.

It is understood that any of the modified chemistries or formats of single stranded oligonucleotides described herein can be combined with each other, and that one, two, three, four, five, or more different types of modifications can be included within the same molecule.

In some embodiments, the method may further comprise the steps of amplifying the synthesized single stranded oligonucleotide, and/or purifying the single stranded oligonucleotide (or amplified single stranded oligonucleotide), and/or sequencing the single stranded oligonucleotide so obtained.

As such, the process of preparing a single stranded oligonucleotide may be a process that is for use in the manufacture of a pharmaceutical composition or medicament for use in the treatment of disease, optionally wherein the treatment involves modulating expression of a target gene.

In the methods described above a lancRNA may be, or have been, identified, or obtained, by a method that involves a detection of the lancRNA. Exemplary methods include RNase protection assays, FISH (fluorescence in situ hybridization), single molecule imaging, deep and/or targeted next generation sequencing. and Northern blots, which are known in the art.

Where the single stranded oligonucleotide is based on a lancRNA sequence, or a portion of such a sequence, it may be based on information about that sequence, e.g., sequence information available in written or electronic form, which may include sequence information contained in publicly available scientific publications or sequence databases.

### Nucleotide Analogues

In some embodiments, the oligonucleotide may comprise at least one ribonucleotide, at least one deoxyribonucleotide, and/or at least one bridged nucleotide. In some embodiments, the oligonucleotide may comprise a bridged nucleotide, such as a locked nucleic acid (LNA) nucleotide, a constrained ethyl (cEt) nucleotide, or an ethylene bridged nucleic acid (ENA) nucleotide. Examples of such nucleotides are disclosed herein and known in the art. In some embodiments, the oligonucleotide comprises a nucleotide analog disclosed in one of the following United States patent or patent application Publications: U.S. Pat. No. 7,399,845, U.S. Pat. No. 7,741,457, U.S. Pat. No. 8,022,193, U.S. Pat. No. 7,569,686, U.S. Pat. No. 7,335,765, U.S. Pat. No. 7,314,923, U.S. Pat. No. 7,335,765, and U.S. Pat. No. 7,816,333, US 20110009471, the entire contents of each of which are incorporated herein by reference for all purposes. The oligonucleotide may have one or more 2′ O-methyl nucleotides. The oligonucleotide may consist entirely of 2′ O-methyl nucleotides.

Often the single stranded oligonucleotide has one or more nucleotide analogues. For example, the single stranded oligonucleotide may have at least one nucleotide analogue that results in an increase in Tₘ of the oligonucleotide in a range of 1° C., 2° C., 3° C., 4° C., or 5° C. compared with an oligonucleotide that does not have the at least one nucleotide analogue. The single stranded oligonucleotide may have a plurality of nucleotide analogues that results in a total increase in Tₘ of the oligonucleotide in a range of 2° C., 3° C., 4° C., 5° C., 6° C., 7° C., 8° C., 9° C., 10° C., 15° C., 20° C., 25° C., 30° C., 35° C., 40° C., 45° C. or more compared with an oligonucleotide that does not have the nucleotide analogue.

The oligonucleotide may be of up to 50 nucleotides in length in which 2 to 10, 2 to 15, 2 to 16, 2 to 17, 2 to 18, 2 to 19, 2 to 20, 2 to 25, 2 to 30, 2 to 40, 2 to 45, or more nucleotides of the oligonucleotide are nucleotide analogues. The oligonucleotide may be of 8 to 30 nucleotides in length in which 2 to 10, 2 to 15, 2 to 16, 2 to 17, 2 to 18, 2 to 19, 2 to 20, 2 to 25, 2 to 30 nucleotides of the oligonucleotide are nucleotide analogues.

The oligonucleotide may be of 8 to 15 nucleotides in length in which 2 to 4, 2 to 5, 2 to 6, 2 to 7, 2 to 8, 2 to 9, 2 to 10, 2 to 11, 2 to 12, 2 to 13, 2 to 14 nucleotides of the oligonucleotide are nucleotide analogues. Optionally, the oligonucleotides may have every nucleotide except 1, 2, 3, 4, 5, 6, 7, 8, 9, or 10 nucleotides modified.

The oligonucleotide may consist entirely of bridged nucleotides (e.g., LNA nucleotides, cEt nucleotides, ENA nucleotides). The oligonucleotide may comprise alternating deoxyribonucleotides and 2′-fluoro-deoxyribonucleotides. The oligonucleotide may comprise alternating deoxyribonucleotides and 2′-O-methyl nucleotides. The oligonucleotide may comprise alternating deoxyribonucleotides and ENA nucleotide analogues. The oligonucleotide may comprise alternating deoxyribonucleotides and LNA nucleotides. The oligonucleotide may comprise alternating LNA nucleotides and 2′-O-methyl nucleotides. The oligonucleotide may have a 5′ nucleotide that is a bridged nucleotide (e.g., a LNA nucleotide, cEt nucleotide, ENA nucleotide). The oligonucleotide may have a 5′ nucleotide that is a deoxyribonucleotide.

The oligonucleotide may comprise deoxyribonucleotides flanked by at least one bridged nucleotide (e.g., a LNA nucleotide, cEt nucleotide, ENA nucleotide) on each of the 5′ and 3′ ends of the deoxyribonucleotides. The oligonucleotide may comprise deoxyribonucleotides flanked by 1, 2, 3, 4, 5, 6, 7, 8 or more bridged nucleotides (e.g., LNA nucleotides, cEt nucleotides, ENA nucleotides) on each of the 5′ and 3′ ends of the deoxyribonucleotides. The 3′ position of the oligonucleotide may have a 3′ hydroxyl group. The 3′ position of the oligonucleotide may have a 3′ thiophosphate.

The oligonucleotide may be conjugated with a label. For example, the oligonucleotide may be conjugated with a biotin moiety, cholesterol, Vitamin A, folate, sigma receptor ligands, aptamers, peptides, such as CPP, hydrophobic molecules, such as lipids, ASGPR or dynamic polyconjugates and variants thereof at its 5′ or 3′ end.

Preferably the single stranded oligonucleotide comprises one or more modifications comprising: a modified sugar moiety, and/or a modified internucleoside linkage, and/or a modified nucleotide and/or combinations thereof. It is not necessary for all positions in a given oligonucleotide to be uniformly modified, and in fact more than one of the modifications described herein may be incorporated in a single oligonucleotide or even at within a single nucleoside within an oligonucleotide.

In some embodiments, the single stranded oligonucleotides are chimeric oligonucleotides that contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric single stranded oligonucleotides of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the single stranded oligonucleotide comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH₂—NH—O—CH₂, CH, ˜N(CH₃)˜O˜CH₂ (known as a methylene(methylimino) or MMI backbone, CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH); amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

Cyclohexenyl nucleic acid oligonucleotide mimetics are described in Wang et al., J. Am. Chem. Soc., 2000, 122, 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264, 562; 5, 264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

Modified oligonucleotides are also known that include oligonucleotides that are based on or constructed from arabinonucleotide or modified arabinonucleotide residues. Arabinonucleosides are stereoisomers of ribonucleosides, differing only in the configuration at the 2′-position of the sugar ring. In some embodiments, a 2′-arabino modification is 2′-F arabino. In some embodiments, the modified oligonucleotide is 2′-fluoro-D-arabinonucleic acid (FANA) (as described in, for example, Lon et al., Biochem., 41:3457-3467, 2002 and Min et al., Bioorg. Med. Chem. Lett., 12:2651-2654, 2002; the disclosures of which are incorporated herein by reference in their entireties). Similar modifications can also be made at other positions on the sugar, particularly the 3′ position of the sugar on a 3′ terminal nucleoside or in 2′-5′ linked oligonucleotides and the 5′ position of 5′ terminal nucleotide.

PCT Publication No. WO 99/67378 discloses arabinonucleic acids (ANA) oligomers and their analogues for improved sequence specific inhibition of gene expression via association to complementary messenger RNA.

Other preferred modifications include ethylene-bridged nucleic acids (ENAs) (e.g., International Patent Publication No. WO 2005/042777, Morita et al., Nucleic Acid Res., Suppl 1:241-242, 2001; Surono et al., Hum. Gene Ther., 15:749-757, 2004; Koizumi, Curr. Opin. Mol. Ther., 8:144-149, 2006 and Horie et al., Nucleic Acids Symp. Ser (Oxf), 49:171-172, 2005; the disclosures of which are incorporated herein by reference in their entireties). Preferred ENAs include, but are not limited to, 2′-0,4′-C-ethylene-bridged nucleic acids.

Examples of LNAs are described in WO/2008/043753 and include compounds of the following general formula.

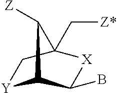

where X and Y are independently selected among the groups —O—,

—S—, —N(H)—, N(R)—, —CH₂— or —CH— (if part of a double bond),

—CH₂—O—, —CH₂—S—, —CH₂—N(H)—, —CH₂—N(R)—, —CH₂—CH₂— or —CH₂—CH— (if part of a double bond),

—CH═CH—, where R is selected from hydrogen and C₁₋₄-alkyl; Z and Z* are independently selected among an internucleoside linkage, a terminal group or a protecting group; B constitutes a natural or non-natural nucleotide base moiety; and the asymmetric groups may be found in either orientation.

Preferably, the LNA used in the oligonucleotides described herein comprises at least one LNA unit according any of the formulas

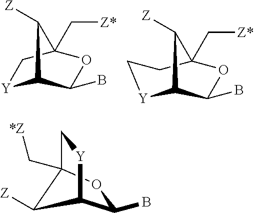

wherein Y is —O—, —S—, —NH—, or N(R^{H}); Z and Z* are independently selected among an internucleoside linkage, a terminal group or a protecting group; B constitutes a natural or non-natural nucleotide base moiety, and RH is selected from hydrogen and C₁₋₄-alkyl.

In some embodiments, the Locked Nucleic Acid (LNA) used in the oligonucleotides described herein comprises at least one Locked Nucleic Acid (LNA) unit according any of the formulas shown in Scheme 2 of PCT/DK2006/000512.

In some embodiments, the LNA used in the oligomer of the invention comprises internucleoside linkages selected from —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —S—P(O)₂—S—, —O—PO(R^{H})—O—, O—PO(OCH₃)—O—, —O—PO(NR^{H})—O—, —O—PO(OCH₂CH₂S—R)—O—, —O—PO(BH₃)—O—, —O—PO(NHR^{H})—O—, —O—P(O)₂—NR^{H}—, —NR^{H}—P(O)₂—O—, —NR^{H}—CO—O—, where R^{H} is selected from hydrogen and C₁₋₄-alkyl.

Specifically preferred LNA units are shown in scheme 2:

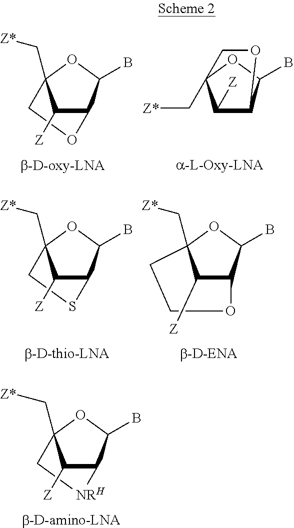

The term “thio-LNA” comprises a locked nucleotide in which at least one of X or Y in the general formula above is selected from S or —CH₂—S—. Thio-LNA can be in both beta-D and alpha-L-configuration.

The term “amino-LNA” comprises a locked nucleotide in which at least one of X or Y in the general formula above is selected from —N(H)—, N(R)—, CH₂—N(H)—, and —CH₂—N(R)— where R is selected from hydrogen and C₁₋₄-alkyl. Amino-LNA can be in both beta-D and alpha-L-configuration.

The term “oxy-LNA” comprises a locked nucleotide in which at least one of X or Y in the general formula above represents —O— or —CH₂—O—. Oxy-LNA can be in both beta-D and alpha-L-configuration.

The term “ena-LNA” comprises a locked nucleotide in which Y in the general formula above is —CH₂—O— (where the oxygen atom of —CH₂—O— is attached to the 2′-position relative to the base B).

LNAs are described in additional detail herein.

One or more substituted sugar moieties can also be included, e.g., one of the following at the 2′ position: OH, SH, SCH₃, F, OCN, OCH₃OCH₃, OCH₃O(CH₂)n CH₃, O(CH₂)n NH₂ or O(CH₂)n CH₃ where n is from 1 to about 10; C1 to C10 lower alkyl, alkoxyalkoxy, substituted lower alkyl, alkaryl or aralkyl; Cl; Br; CN; CF₃; OCF₃; O-, S-, or N-alkyl; O-, S-, or N-alkenyl; SOCH₃; SO₂CH₃; ONO₂; NO₂; N₃; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-O—CH₂CH₂OCH₃, also known as 2′-O-(2-methoxyethyl)] (Martin et al, HeIv. Chim. Acta, 1995, 78, 486). Other preferred modifications include 2′-methoxy (2′-O—CH₃), 2′-propoxy (2′-OCH₂CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Single stranded oligonucleotides can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, isocytosine, pseudoisocytosine, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 5-propynyluracil, 8-azaguanine, 7-deazaguanine, N6 (6-aminohexyl)adenine, 6-aminopurine, 2-aminopurine, 2-chloro-6-aminopurine and 2,6-diaminopurine or other diaminopurines. See, e.g., Kornberg, “DNA Replication,” W. H. Freeman & Co., San Francisco, 1980, pp 75-′7′7; and Gebeyehu, G., et al. Nucl. Acids Res., 15:4513 (1987)). A “universal” base known in the art, e.g., inosine, can also be included. 5-Me-C substitutions have been shown to increase nucleic acid duplex stability by 0.6-1.2° C. (Sanghvi, in Crooke, and Lebleu, eds., Antisense Research and Applications, CRC Press, Boca Raton, 1993, pp. 276-278) and may be used as base substitutions.

It is not necessary for all positions in a given oligonucleotide to be uniformly modified, and in fact more than one of the modifications described herein may be incorporated in a single oligonucleotide or even at within a single nucleoside within an oligonucleotide.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Single stranded oligonucleotides can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine. In some embodiments, a cytosine is substituted with a 5-methylcytosine. In some embodiments, an oligonucleotide has 2, 3, 4, 5, 6, 7, or more cytosines substituted with 5-methylcytosines. In some embodiments, an oligonucleotide does not have 2, 3, 4, 5, 6, 7, or more consecutive 5-methylcytosines. In some embodiments, an LNA cytosine nucleotide is replaced with an LNA 5-methylcytosine nucleotide.

Further, nucleobases comprise those disclosed in U.S. Pat. No. 3,687,808, those disclosed in “The Concise Encyclopedia of Polymer Science And Engineering”, pages 858-859, Kroschwitz, ed. John Wiley & Sons, 1990; those disclosed by Englisch et al., Angewandle Chemie, International Edition, 1991, 30, page 613, and those disclosed by Sanghvi, Chapter 15, Antisense Research and Applications,” pages 289-302, Crooke, and Lebleu, eds., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by 0.6-1.2<0>C (Sanghvi, et al., eds, “Antisense Research and Applications,” CRC Press, Boca Raton, 1993, pp. 276-278) and are presently preferred base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175, 273; 5, 367,066; 5,432,272; 5,457,187; 5,459,255; 5,484,908; 5,502,177; 5,525,711; 5,552,540; 5,587,469; 5,596,091; 5,614,617; 5,750,692, and 5,681,941, each of which is herein incorporated by reference.

In some embodiments, the single stranded oligonucleotides are chemically linked to one or more moieties or conjugates that enhance the activity, cellular distribution, or cellular uptake of the oligonucleotide. For example, one or more single stranded oligonucleotides, of the same or different types, can be conjugated to each other; or single stranded oligonucleotides can be conjugated to targeting moieties with enhanced specificity for a cell type or tissue type. Such moieties include, but are not limited to, lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al, Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-t oxycholesterol moiety (Crooke et al., J. Pharmacol. Exp. Ther., 1996, 277, 923-937). See also U.S. Pat. Nos. 4,828,979; 4,948,882; 5,218,105; 5,525,465; 5,541,313; 5,545,730; 5,552, 538; 5,578,717, 5,580,731; 5,580,731; 5,591,584; 5,109,124; 5,118,802; 5,138,045; 5,414,077; 5,486, 603; 5,512,439; 5,578,718; 5,608,046; 4,587,044; 4,605,735; 4,667,025; 4,762, 779; 4,789,737; 4,824,941; 4,835,263; 4,876,335; 4,904,582; 4,958,013; 5,082, 830; 5,112,963; 5,214,136; 5,082,830; 5,112,963; 5,214,136; 5, 245,022; 5,254,469; 5,258,506; 5,262,536; 5,272,250; 5,292,873; 5,317,098; 5,371,241, 5,391, 723; 5,416,203, 5,451,463; 5,510,475; 5,512,667; 5,514,785; 5, 565,552; 5,567,810; 5,574,142; 5,585,481; 5,587,371; 5,595,726; 5,597,696; 5,599,923; 5,599, 928 and 5,688,941, each of which is herein incorporated by reference.

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

In some embodiments, single stranded oligonucleotide modification include modification of the 5′ or 3′ end of the oligonucleotide. In some embodiments, the 3′ end of the oligonucleotide comprises a hydroxyl group or a thiophosphate. It should be appreciated that additional molecules (e.g. a biotin moiety or a fluorophor) can be conjugated to the 5′ or 3′ end of the single stranded oligonucleotide. In some embodiments, the single stranded oligonucleotide comprises a biotin moiety conjugated to the 5′ nucleotide.

In some embodiments, the single stranded oligonucleotide comprises locked nucleic acids (LNA), ENA modified nucleotides, 2′-O-methyl nucleotides, or 2′-fluoro-deoxyribonucleotides. In some embodiments, the single stranded oligonucleotide comprises alternating deoxyribonucleotides and 2′-fluoro-deoxyribonucleotides. In some embodiments, the single stranded oligonucleotide comprises alternating deoxyribonucleotides and 2′-O-methyl nucleotides. In some embodiments, the single stranded oligonucleotide comprises alternating deoxyribonucleotides and ENA modified nucleotides. In some embodiments, the single stranded oligonucleotide comprises alternating deoxyribonucleotides and locked nucleic acid nucleotides. In some embodiments, the single stranded oligonucleotide comprises alternating locked nucleic acid nucleotides and 2′-O-methyl nucleotides.

In some embodiments, the 5′ nucleotide of the oligonucleotide is a deoxyribonucleotide. In some embodiments, the 5′ nucleotide of the oligonucleotide is a locked nucleic acid nucleotide. In some embodiments, the nucleotides of the oligonucleotide comprise deoxyribonucleotides flanked by at least one locked nucleic acid nucleotide on each of the 5′ and 3′ ends of the deoxyribonucleotides. In some embodiments, the nucleotide at the 3′ position of the oligonucleotide has a 3′ hydroxyl group or a 3′ thiophosphate.

In some embodiments, the single stranded oligonucleotide comprises phosphorothioate internucleotide linkages. In some embodiments, the single stranded oligonucleotide comprises phosphorothioate internucleotide linkages between at least two nucleotides. In some embodiments, the single stranded oligonucleotide comprises phosphorothioate internucleotide linkages between all nucleotides.

It should be appreciated that the single stranded oligonucleotide can have any combination of modifications as described herein.

In some embodiments, an oligonucleotide described herein may be a mixmer or comprise a mixmer sequence pattern. The term ‘mixmer’ refers to oligonucleotides which comprise both naturally and non-naturally occurring nucleotides or comprise two different types of non-naturally occurring nucleotides. Mixmers are generally known in the art to have a higher binding affinity than unmodified oligonucleotides and may be used to specifically bind a target molecule, e.g., to block a binding site on the target molecule. Generally, mixmers do not recruit an RNAse to the target molecule and thus do not promote cleavage of the target molecule. Accordingly, in some embodiments, an oligonucleotide provided herein may be cleavage promoting (e.g., an siRNA or gapmer) or not cleavage promoting (e.g., a mixmer, siRNA, single stranded RNA or double stranded RNA).

In some embodiments, the mixmer comprises or consists of a repeating pattern of nucleotide analogues and naturally occurring nucleotides, or one type of nucleotide analogue and a second type of nucleotide analogue. However, it is to be understood that the mixmer need not comprise a repeating pattern and may instead comprise any arrangement of nucleotide analogues and naturally occurring nucleotides or any arrangement of one type of nucleotide analogue and a second type of nucleotide analogue. The repeating pattern, may, for instance be every second or every third nucleotide is a nucleotide analogue, such as LNA, and the remaining nucleotides are naturally occurring nucleotides, such as DNA, or are a 2′ substituted nucleotide analogue such as 2′MOE or 2′ fluoro analogues, or any other nucleotide analogues described herein. It is recognised that the repeating pattern of nucleotide analogues, such as LNA units, may be combined with nucleotide analogues at fixed positions—e.g. at the 5′ or 3′ termini.

In some embodiments, the mixmer does not comprise a region of more than 5, more than 4, more than 3, or more than 2 consecutive naturally occurring nucleotides, such as DNA nucleotides. In some embodiments, the mixmer comprises at least a region consisting of at least two consecutive nucleotide analogues, such as at least two consecutive LNAs. In some embodiments, the mixmer comprises at least a region consisting of at least three consecutive nucleotide analogue units, such as at least three consecutive LNAs.

In some embodiments, the mixmer does not comprise a region of more than 7, more than 6, more than 5, more than 4, more than 3, or more than 2 consecutive nucleotide analogues, such as LNAs. It is to be understood that the LNA units may be replaced with other nucleotide analogues, such as those referred to herein.

In some embodiments, the mixmer comprises at least one nucleotide analogue in one or more of six consecutive nucleotides. The substitution pattern for the nucleotides may be selected from the group consisting of Xxxxxx, xXxxxx, xxXxxx, xxxXxx, xxxxXx and xxxxxX, wherein “X” denotes a nucleotide analogue, such as an LNA, and “x” denotes a naturally occurring nucleotide, such as DNA or RNA.

In some embodiments, the mixmer comprises at least two nucleotide analogues in one or more of six consecutive nucleotides. The substitution pattern for the nucleotides may be selected from the group consisting of XXxxxx, XxXxxx, XxxXxx, XxxxXx, XxxxxX, xXXxxx, xXxXxx, xXxxXx, xXxxxX, xxXXxx, xxXxXx, xxXxxX, xxxXXx, xxxXxX and xxxxXX, wherein “X” denotes a nucleotide analogue, such as an LNA, and “x” denotes a naturally occurring nucleotide, such as DNA or RNA. In some embodiments, the substitution pattern for the nucleotides may be selected from the group consisting of XxXxxx, XxxXxx, XxxxXx, XxxxxX, xXxXxx, xXxxXx, xXxxxX, xxXxXx, xxXxxX and xxxXxX. In some embodiments, the substitution pattern is selected from the group consisting of xXxXxx, xXxxXx, xXxxxX, xxXxXx, xxXxxX and xxxXxX. In some embodiments, the substitution pattern is selected from the group consisting of xXxXxx, xXxxXx and xxXxXx. In some embodiments, the substitution pattern for the nucleotides is xXxXxx.

In some embodiments, the mixmer comprises at least three nucleotide analogues in one or more of six consecutive nucleotides. The substitution pattern for the nucleotides may be selected from the group consisting of XXXxxx, xXXXxx, xxXXXx, xxxXXX, XXxXxx, XXxxXx, XXxxxX, xXXxXx, xXXxxX, xxXXxX, XxXXxx, XxxXXx, XxxxXX, xXxXXx, xXxxXX, xxXxXX, xXxXxX and XxXxXx, wherein “X” denotes a nucleotide analogue, such as an LNA, and “x” denotes a naturally occuring nucleotide, such as DNA or RNA. In some embodiments, the substitution pattern for the nucleotides is selected from the group consisting of XXxXxx, XXxxXx, XXxxxX, xXXxXx, xXXxxX, xxXXxX, XxXXxx, XxxXXx, XxxxXX, xXxXXx, xXxxXX, xxXxXX, xXxXxX and XxXxXx. In some embodiments, the substitution pattern for the nucleotides is selected from the group consisting of xXXxXx, xXXxxX, xxXXxX, xXxXXx, xXxxXX, xxXxXX and xXxXxX. n some embodiments, the substitution pattern for the nucleotides is xXxXxX or XxXxXx. In some embodiments, the substitution pattern for the nucleotides is xXxXxX.

In some embodiments, the mixmer comprises at least four nucleotide analogues in one or more of six consecutive nucleotides. The substitution pattern for the nucleotides may be selected from the group consisting of xXXXX, xXxXXX, xXXxXX, xXXXxX, xXXXXx, XxxXXX, XxXxXX, XxXXxX, XxXXXx, XXxxXX, XXxXxX, XXxXXx, XXXxxX, XXXxXx and XXXXxx, wherein “X” denotes a nucleotide analogue, such as an LNA, and “x” denotes a naturally occurring nucleotide, such as DNA or RNA.

In some embodiments, the mixmer comprises at least five nucleotide analogues in one or more of six consecutive nucleotides. The substitution pattern for the nucleotides may be selected from the group consisting of xXXXXX, XxXXXX, XXxXXX, XXXxXX, XXXXxX and XXXXXx, wherein “X” denotes a nucleotide analogue, such as an LNA, and “x” denotes a naturally occurring nucleotide, such as DNA or RNA.

The oligonucleotide may comprise a nucleotide sequence having one or more of the following modification patterns.

(a) (X)Xxxxxx, (X)xXxxxx, (X)xxXxxx, (X)xxxXxx, (X)xxxxXx and (X)xxxxxX,

(b) (X)XXxxxx, (X)XxXxxx, (X)XxxXxx, (X)XxxxXx, (X)XxxxxX, (X)xXXxxx, (X)xXxXxx, (X)xXxxXx, (X)xXxxxX, (X)xxXXxx, (X)xxXxXx, (X)xxXxxX, (X)xxxXXx, (X)xxxXxX and (X)xxxxXX,

(c) (X)XXXxxx, (X)xXXXxx, (X)xxXXXx, (X)xxxXXX, (X)XXxXxx, (X)XXxxXx, (X)XXxxxX, (X)xXXxXx, (X)xXXxxX, (X)xxXXxX, (X)XxXXxx, (X)XxxXXx (X)XxxxXX, (X)xXxXXx, (X)xXxxXX, (X)xxXxXX, (X)xXxXxX and (X)XxXxXx,

(d) (X)xxXXX, (X)xXxXXX, (X)xXXxXX, (X)xXXXxX, (X)xXXXXx, (X)XxxXXXX, (X)XxXxXX, (X)XxXXxX, (X)XxXXx, (X)XXxxXX, (X)XXxXxX, (X)XXxXXx, (X)XXXxxX, (X)XXXxXx, and (X)XXXXxx,

(e) (X)xXXXXX, (X)XxXXXX, (X)XXxXXX, (X)XXXxXX, (X)XXXXxX and (X)XXXXXx, and

(f) XXXXXX, XxXXXXX, XXxXXXX, XXXxXXX, XXXXxXX, XXXXXxX and XXXXXXx, in which “X” denotes a nucleotide analogue, (X) denotes an optional nucleotide analogue, and “x” denotes a DNA or RNA nucleotide unit. Each of the above listed patterns may appear one or more times within an oligonucleotide, alone or in combination with any of the other disclosed modification patterns.

In some embodiments, the mixmer contains a modified nucleotide, e.g., an LNA, at the 5′ end. In some embodiments, the mixmer contains a modified nucleotide, e.g., an LNA, at the first two positions, counting from the 5′ end.

In some embodiments, the mixmer is incapable of recruiting RNAseH. Oligonucleotides that are incapable of recruiting RNAseH are well known in the literature, in example see WO2007/112754, WO2007/112753, or PCT/DK2008/000344. Mixmers may be designed to comprise a mixture of affinity enhancing nucleotide analogues, such as in non-limiting example LNA nucleotides and 2′-O-methyl nucleotides. In some embodiments, the mixmer comprises modified internucleoside linkages (e.g., phosphorothioate internucleoside linkages or other linkages) between at least two, at least three, at least four, at least five or more nucleotides.

A mixmer may be produced using any method known in the art or described herein. Representative U.S. patents, U.S. patent publications, and PCT publications that teach the preparation of mixmers include U.S. patent publication Nos. US20060128646, US20090209748, US20090298916, US20110077288, and US20120322851, and U.S. Pat. No. 7,687,617.

In some embodiments, the oligonucleotide is a gapmer. A gapmer oligonucleotide generally has the formula 5′-X-Y-Z-3′, with X and Z as flanking regions around a gap region Y. In some embodiments, the Y region is a contiguous stretch of nucleotides, e.g., a region of at least 6 DNA nucleotides, which are capable of recruiting an RNAse, such as RNAseH. Without wishing to be bound by theory, it is thought that the gapmer binds to the target nucleic acid, at which point an RNAse is recruited and can then cleave the target nucleic acid. In some embodiments, the Y region is flanked both 5′ and 3′ by regions X and Z comprising high-affinity modified nucleotides, e.g., 1-6 modified nucleotides. Exemplary modified oligonucleotides include, but are not limited to, 2′ MOE or 2′OMe or Locked Nucleic Acid bases (LNA). The flanks X and Z may be have a of length 1-20 nucleotides, preferably 1-8 nucleotides and even more preferred 1-5 nucleotides. The flanks X and Z may be of similar length or of dissimilar lengths. The gap-segment Y may be a nucleotide sequence of length 5-20 nucleotides, preferably 6-12 nucleotides and even more preferred 6-10 nucleotides. In some aspects, the gap region of the gapmer oligonucleotides of the invention may contain modified nucleotides known to be acceptable for efficient RNase H action in addition to DNA nucleotides, such as C4′-substituted nucleotides, acyclic nucleotides, and arabino-configured nucleotides. In some embodiments, the gap region comprises one or more unmodified internucleosides. In some embodiments, one or both flanking regions each independently comprise one or more phosphorothioate internucleoside linkages (e.g., phosphorothioate internucleoside linkages or other linkages) between at least two, at least three, at least four, at least five or more nucleotides. In some embodiments, the gap region and two flanking regions each independently comprise modified internucleoside linkages (e.g., phosphorothioate internucleoside linkages or other linkages) between at least two, at least three, at least four, at least five or more nucleotides.

A gapmer may be produced using any method known in the art or described herein. Representative U.S. patents, U.S. patent publications, and PCT publications that teach the preparation of gapmers include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; 5,700,922; 5,898,031; 7,432,250; and 7,683,036; U.S. patent publication Nos. US20090286969, US20100197762, and US20110112170; and PCT publication Nos. WO2008049085 and WO2009090182, each of which is herein incorporated by reference in its entirety.

In some embodiments, an oligonucleotide described herein comprises a synthetic cap, e.g., to increase efficiency of translation, RNA half-life and/or function within cells. Synthetic caps are known in the art. Exemplary synthetic caps include, but are not limited to, N7-Methyl-Guanosine-5′-Triphosphate-5′-Guanosine, Guanosine-5′-Triphosphate-5*-Guanosine, N7-Methyl-3′-O-Methyl-Guanosine-5′-Triphosphate-5′-Guanosine (see, e.g., products available from TrilinkBiotech), and N7-benzylated dinucleoside tetraphosphate analogs (see, e.g., Grudzien et al. Novel cap analogs for in vitro synthesis of mRNAs with high translational efficiency. RNA. 2004 September; 10(9): 1479-1487).

### Methods for Modulating Gene Expression

In one aspect, the invention relates to methods for modulating (e.g., upregulating or downregulating) gene expression in a cell (e.g., a cell for which levels of the target gene are reduced or enhanced) for research purposes (e.g., to study the function of the gene in the cell). In another aspect, the invention relates to methods for modulating gene expression in a cell (e.g., a cell for which levels of the target gene are reduced or enhanced) for gene or epigenetic therapy. The cells can be in vitro, ex vivo, or in vivo (e.g., in a subject who has a disease resulting from reduced expression or activity of a target gene. In some embodiments, methods for modulating gene expression in a cell comprise delivering a single stranded oligonucleotide as described herein. In some embodiments, delivery of the single stranded oligonucleotide to the cell results in a level of expression of gene that is at least 5%, 10%, 20%, 30%, 40%, 50%, 60%, 70%, 80%, 90%, 100%, 200% or more greater than a level of expression of gene in a control cell to which the single stranded oligonucleotide has not been delivered. In certain embodiments, delivery of the single stranded oligonucleotide to the cell results in a level of expression of gene that is at least 50% greater than a level of expression of gene in a control cell to which the single stranded oligonucleotide has not been delivered. In some embodiments, delivery of the single stranded oligonucleotide to the cell results in a level of expression of gene that is at least 5%, 10%, 20%, 30%, 40%, 50%, 60%, 70%, 80%, 90%, 100%, 200% or more less than a level of expression of gene in a control cell to which the single stranded oligonucleotide has not been delivered.

In another aspect of the invention, methods comprise administering to a subject (e.g. a human) a composition comprising a single stranded oligonucleotide as described herein to increase protein levels in the subject. In some embodiments, the increase in protein levels is at least 5%, 10%, 20%, 30%, 40%, 50%, 60%, 70%, 80%, 90%, 100%, 200%, or more, higher than the amount of a protein in the subject before administering.

In another aspect of the invention, methods comprise administering to a subject (e.g. a human) a composition comprising a single stranded oligonucleotide as described herein to decrease protein levels in the subject. In some embodiments, the decrease in protein levels is a decrease of at least 5%, 10%, 20%, 30%, 40%, 50%, 60%, 70%, 80%, 90%, 100%, 200%, or more, compared to the amount of a protein in the subject before administering.

As another example, to increase or decrease expression of a target gene in a cell, the methods include introducing into the cell a single stranded oligonucleotide that is sufficiently complementary to a lancRNA that maps to a genomic position encompassing or in proximity to a transcriptional boundary of the target gene.

In another aspect of the invention provides methods of treating a disease or condition associated with decreased levels of expression of a target gene in a subject, the method comprising administering a single stranded oligonucleotide as described herein. Exemplary diseases and condition associated with certain genes are provided in Table 2.

TABLE 2
Examples of diseases or conditions treatable with oligonucleotides
targeting lancRNAs associated with genes.

| Gene | Disease of conditions |
|---|---|
| FXN | Friedreich's Ataxia |
| SMN | Spinal muscular atrophy (SMA) types I-IV |
| UTRN | Muscular dystrophy (MD) (e.g., Duchenne's muscular dystrophy, |
| | Becker's muscular dystrophy, myotonic dystrophy) |
| HEMOGLOBIN | Anemia, microcytic anemia, sickle cell anemia and/or thalassemia (e.g., |
| | alpha-thalassemia, beta-thalaseemia, delta-thalessemia), beta-thalaseemia |
| | (e.g., thalassemia minor/intermedia/major) |
| ATP2A2 | Cardiac conditions (e.g., congenital heart disease, aortic aneurysms, |
| | aortic dissections, arrhythmia, cardiomyopathy, and congestive heart |
| | failure), Darier-White disease and Acrokeratosis verruciformi |
| APOA1/ | Dyslipidemia (e.g. Hyperlipidemia) and atherosclerosis (e.g. coronary |
| ABCA1 | artery disease (CAD) and myocardial infarction (MI)) |
| PTEN | Cancer, such as, leukemias, lymphomas, myelomas, carcinomas, |
| | metastatic carcinomas, sarcomas, adenomas, nervous system cancers and |
| | genito-urinary cancers. In some embodiments, the cancer is adult and |
| | pediatric acute lymphoblastic leukemia, acute myeloid leukemia, |
| | adrenocortical carcinoma, AIDS-related cancers, anal cancer, cancer of |
| | the appendix, astrocytoma, basal cell carcinoma, bile duct cancer, |
| | bladder cancer, bone cancer, osteosarcoma, fibrous histiocytoma, brain |
| | cancer, brain stem glioma, cerebellar astrocytoma, malignant glioma, |
| | ependymoma, medulloblastoma, supratentorial primitive |
| | neuroectodermal tumors, hypothalamic glioma, breast cancer, male |
| | breast cancer, bronchial adenomas, Burkitt lymphoma, carcinoid tumor, |
| | carcinoma of unknown origin, central nervous system lymphoma, |
| | cerebellar astrocytoma, malignant glioma, cervical cancer, childhood |
| | cancers, chronic lymphocytic leukemia, chronic myelogenous leukemia, |
| | chronic myeloproliferative disorders, colorectal cancer, cutaneous T-cell |
| | lymphoma, endometrial cancer, ependymoma, esophageal cancer, Ewing |
| | family tumors, extracranial germ cell tumor, extragonadal germ cell |
| | tumor, extrahepatic bile duct cancer, intraocular melanoma, |
| | retinoblastoma, gallbladder cancer, gastric cancer, gastrointestinal |
| | stromal tumor, extracranial germ cell tumor, extragonadal germ cell |
| | tumor, ovarian germ cell tumor, gestational trophoblastic tumor, glioma, |
| | hairy cell leukemia, head and neck cancer, hepatocellular cancer, |
| | Hodgkin lymphoma, non-Hodgkin lymphoma, hypopharyngeal cancer, |
| | hypothalamic and visual pathway glioma, intraocular melanoma, islet |
| | cell tumors, Kaposi sarcoma, kidney cancer, renal cell cancer, laryngeal |
| | cancer, lip and oral cavity cancer, small cell lung cancer, non-small cell |
| | lung cancer, primary central nervous system lymphoma, Waldenstrom |
| | macroglobulinema, malignant fibrous histiocytoma, medulloblastoma, |
| | melanoma, Merkel cell carcinoma, malignant mesothelioma, squamous |
| | neck cancer, multiple endocrine neoplasia syndrome, multiple myeloma, |
| | mycosis fungoides, myelodysplastic syndromes, myeloproliferative |
| | disorders, chronic myeloproliferative disorders, nasal cavity and |
| | paranasal sinus cancer, nasopharyngeal cancer, neuroblastoma, |
| | oropharyngeal cancer, ovarian cancer, pancreatic cancer, parathyroid |
| | cancer, penile cancer, pharyngeal cancer, pheochromocytoma, |
| | pineoblastoma and supratentorial primitive neuroectodermal tumors, |
| | pituitary cancer, plasma cell neoplasms, pleuropulmonary blastoma, |
| | prostate cancer, rectal cancer, rhabdomyosarcoma, salivary gland cancer, |
| | soft tissue sarcoma, uterine sarcoma, Sezary syndrome, non-melanoma |
| | skin cancer, small intestine cancer, squamous cell carcinoma, squamous |
| | neck cancer, supratentorial primitive neuroectodermal tumors, testicular |
| | cancer, throat cancer, thymoma and thymic carcinoma, thyroid cancer, |
| | transitional cell cancer, trophoblastic tumors, urethral cancer, uterine |
| | cancer, uterine sarcoma, vaginal cancer, vulvar cancer, or Wilms tumor |
| BDNF | Amyotrophic lateral sclerosis (ALS, also known as Lou Gehrig's |
| | disease), Alzheimer's Disease (AD), and Parkinson's Disease (PD), |
| | Neurodegeneration |
| MECP2 | Rett Syndrome, MECP2-related severe neonatal encephalopathy, |
| | Angelman syndrome, or PPM-X syndrome |
| FOXP3 | Diseases or disorders associated with aberrant immune cell (e.g., T cell) |
| | activation, e.g., autoimmune or inflammatory diseases or disorders. |
| | Examples of autoimmune diseases and disorders that may be treated |
| | according to the methods disclosed herein include, but are not limited to, |
| | Acute Disseminated Encephalomyelitis (ADEM), Acute necrotizing |
| | hemorrhagic leukoencephalitis, Addison's disease, |
| | Agammaglobulinemia, Alopecia areata, Amyloidosis, Ankylosing |
| | spondylitis, Anti-GBM/Anti-TBM nephritis, Antiphospholipid syndrome |
| | (APS), Autoimmune angioedema, Autoimmune aplastic anemia, |
| | Autoimmune dysautonomia, Autoimmune hepatitis, Autoimmune |
| | hyperlipidemia, Autoimmune immunodeficiency, Autoimmune inner ear |
| | disease (AIED), Autoimmune myocarditis, Autoimmune oophoritis, |
| | Autoimmune pancreatitis, Autoimmune retinopathy, Autoimmune |
| | thrombocytopenic purpura (ATP), Autoimmune thyroid disease, |
| | Autoimmune urticaria, Axonal & neuronal neuropathies, Balo disease, |
| | Behcet's disease, Bullous pemphigoid, Cardiomyopathy, Castleman |
| | disease, Celiac disease, Chagas disease, Chronic inflammatory |
| | demyelinating polyneuropathy (CIDP), Chronic recurrent multifocal |
| | ostomyelitis (CRMO), Churg-Strauss syndrome, Cicatricial |
| | pemphigoid/benign mucosal pemphigoid, inflammatory bowel disease |
| | (e.g., Crohn's disease or Ulcerative colitis), Cogans syndrome, Cold |
| | agglutinin disease, Congenital heart block, Coxsackie myocarditis, |
| | CREST disease, Essential mixed cryoglobulinemia, Demyelinating |
| | neuropathies, Dermatitis herpetiformis, Dermatomyositis, Devic's |
| | disease (neuromyelitis optica), Discoid lupus, Dressler's syndrome, |
| | Endometriosis, Eosinophilic esophagitis, Eosinophilic fasciitis, Erythema |
| | nodosum, Experimental allergic encephalomyelitis, Evans syndrome, |
| | Fibrosing alveolitis, Giant cell arteritis (temporal arteritis), Giant cell |
| | myocarditis, Glomerulonephritis, Goodpasture's syndrome, |
| | Granulomatosis with Polyangiitis (GPA) (formerly called Wegener's |
| | Granulomatosis), Graves' disease, Guillain-Barre syndrome, Hashimoto's |
| | encephalitis, Hashimoto's thyroiditis, Hemolytic anemia, Henoch- |
| | Schonlein purpura, Herpes gestationis, Hypogammaglobulinemia, |
| | Idiopathic thrombocytopenic purpura (ITP), IgA nephropathy, IgG4- |
| | related sclerosing disease, Immunoregulatory lipoproteins, Inclusion |
| | body myositis, Interstitial cystitis, IPEX (Immunodysregulation, |
| | Polyendocrinopathy, and Enteropathy, X-linked) syndrome, Juvenile |
| | arthritis, Juvenile diabetes (Type 1 diabetes), Juvenile myositis, |
| | Kawasaki syndrome, Lambert-Eaton syndrome, Leukocytoclastic |
| | vasculitis, Lichen planus, Lichen sclerosus, Ligneous conjunctivitis, |
| | Linear IgA disease (LAD), systemic lupus erythematosus (SLE), chronic |
| | Lyme disease, Meniere's disease, Microscopic polyangiitis, Mixed |
| | connective tissue disease (MCTD), Mooren's ulcer, Mucha-Habermann |
| | disease, Multiple sclerosis, Myasthenia gravis, Myositis, Narcolepsy, |
| | Neuromyelitis optica (Devic's), Neutropenia, Ocular cicatricial |
| | pemphigoid, Optic neuritis, Palindromic rheumatism, PANDAS |
| | (Pediatric Autoimmune Neuropsychiatric Disorders Associated with |
| | Streptococcus), Paraneoplastic cerebellar degeneration, Paroxysmal |
| | nocturnal hemoglobinuria (PNH), Parry Romberg syndrome, |
| | Parsonnage-Turner syndrome, Pars planitis (peripheral uveitis), |
| | Pemphigus, Peripheral neuropathy, Perivenous encephalomyelitis, |
| | Pernicious anemia, POEMS syndrome, Polyarteritis nodosa, Type I, II, |
| | & III autoimmune polyglandular syndromes, Polymyalgia rheumatica, |
| | Polymyositis, Postmyocardial infarction syndrome, Postpericardiotomy |
| | syndrome, Progesterone dermatitis, Primary biliary cirrhosis, Primary |
| | sclerosing cholangitis, Psoriasis, Psoriatic arthritis, Idiopathic pulmonary |
| | fibrosis, Pyoderma gangrenosum, Pure red cell aplasia, Raynauds |
| | phenomenon, Reactive Arthritis, Reflex sympathetic dystrophy, Reiter's |
| | syndrome, Relapsing polychondritis, Restless legs syndrome, |
| | Retroperitoneal fibrosis, Rheumatic fever, Rheumatoid arthritis, |
| | Sarcoidosis, Schmidt syndrome, Scleritis, Scleroderma, Sjogren's |
| | syndrome, Sperm & testicular autoimmunity, Stiff person syndrome, |
| | Subacute bacterial endocarditis (SBE), Susac's syndrome, Sympathetic |
| | ophthalmia, Takayasu's arteritis, Temporal arteritis/Giant cell arteritis, |
| | Thrombocytopenic purpura (TTP), Tolosa-Hunt syndrome, Transverse |
| | myelitis, Type 1 diabetes, Undifferentiated connective tissue disease |
| | (UCTD), Uveitis, Vasculitis, Vesiculobullous dermatosis, Vitiligo, and |
| | Wegener's granulomatosis (also called Granulomatosis with Polyangiitis |
| | (GPA)). Further examples of autoimmune disease or disorder include |
| | inflammatory bowel disease (e.g., Crohn's disease or Ulcerative colitis), |
| | IPEX syndrome, Multiple sclerosis, Psoriasis, Rheumatoid arthritis, SLE |
| | or Type 1 diabetes. Examples of inflammatory diseases or disorders that |
| | may be treated according to the methods disclosed herein include, but are |
| | not limited to, Acne Vulgaris, Appendicitis, Arthritis, Asthma, |
| | Atherosclerosis, Allergies (Type 1 Hypersensitivity), Bursitis, Colitis, |
| | Chronic Prostatitis, Cystitis, Dermatitis, Glomerulonephritis, |
| | Inflammatory Bowel Disease, Inflammatory Myopathy (e.g., |
| | Polymyositis, Dermatomyositis, or Inclusion-body Myositis), |
| | Inflammatory Lung Disease, Interstitial Cystitis, Meningitis, Pelvic |
| | Inflammatory Disease, Phlebitis, Psoriasis, Reperfusion Injury, |
| | Rheumatoid Arthritis, Sarcoidosis, Tendonitis, Tonsilitis, Transplant |
| | Rejection, and Vasculitis. In some embodiments, the inflammatory |
| | disease or disorder is asthma. |
| THRB | Thyroid hormone resistance, mixed dyslipidemia, dyslipidemia, |
| | hypercholesterolemia |
| NR1H4 | Byler disease, cholestasis, cholestasis intrahepatic, dyslipidemia, biliary |
| | cirrhosis primary, fragile x syndrome, hypercholesterolemia, |
| | atherosclerosis, biliary atresia |
| HAMP | Hemochromatosis (juvenile), hemochromatosis , iron overload, |
| | hereditary hemochromatosis, anemia, inflammation, thalassemia |

A subject can include a non-human mammal, e.g. mouse, rat, guinea pig, rabbit, cat, dog, goat, cow, or horse. In preferred embodiments, a subject is a human. Single stranded oligonucleotides have been employed as therapeutic moieties in the treatment of disease states in animals, including humans. Single stranded oligonucleotides can be useful therapeutic modalities that can be configured to be useful in treatment regimens for the treatment of cells, tissues and animals, especially humans.

For therapeutics, an animal, preferably a human, suspected of having a disease or condition is treated by administering single stranded oligonucleotide in accordance with this invention. For example, in one non-limiting embodiment, the methods comprise the step of administering to the animal in need of treatment, a therapeutically effective amount of a single stranded oligonucleotide as described herein.

### Formulation, Delivery, and Dosing

The oligonucleotides described herein can be formulated for administration to a subject for treating a condition or disease associated with increased or decreased levels of a target gene. It should be understood that the formulations, compositions and methods can be practiced with any of the oligonucleotides disclosed herein.

The formulations may conveniently be presented in unit dosage form and may be prepared by any methods well known in the art of pharmacy. The amount of active ingredient (e.g., an oligonucleotide or compound of the invention) which can be combined with a carrier material to produce a single dosage form will vary depending upon the host being treated, the particular mode of administration, e.g., intradermal or inhalation. The amount of active ingredient which can be combined with a carrier material to produce a single dosage form will generally be that amount of the compound which produces a therapeutic effect, e.g. tumor regression.

Pharmaceutical formulations of this invention can be prepared according to any method known to the art for the manufacture of pharmaceuticals. Such formulations can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

A formulated single stranded oligonucleotide composition can assume a variety of states. In some examples, the composition is at least partially crystalline, uniformly crystalline, and/or anhydrous (e.g., less than 80, 50, 30, 20, or 10% water). In another example, the single stranded oligonucleotide is in an aqueous phase, e.g., in a solution that includes water. The aqueous phase or the crystalline compositions can, e.g., be incorporated into a delivery vehicle, e.g., a liposome (particularly for the aqueous phase) or a particle (e.g., a microparticle as can be appropriate for a crystalline composition). Generally, the single stranded oligonucleotide composition is formulated in a manner that is compatible with the intended method of administration.

In some embodiments, the composition is prepared by at least one of the following methods: spray drying, lyophilization, vacuum drying, evaporation, fluid bed drying, or a combination of these techniques; or sonication with a lipid, freeze-drying, condensation and other self-assembly.

A single stranded oligonucleotide preparation can be formulated or administered (together or separately) in combination with another agent, e.g., another therapeutic agent or an agent that stabilizes a single stranded oligonucleotide, e.g., a protein that complexes with single stranded oligonucleotide. Still other agents include chelators, e.g., EDTA (e.g., to remove divalent cations such as Mg²⁺), salts, RNAse inhibitors (e.g., a broad specificity RNAse inhibitor such as RNAsin) and so forth.

In one embodiment, the single stranded oligonucleotide preparation includes another single stranded oligonucleotide, e.g., a second single stranded oligonucleotide that modulates expression of a second gene or a second single stranded oligonucleotide that modulates expression of the first gene. Still other preparation can include at least 3, 5, ten, twenty, fifty, or a hundred or more different single stranded oligonucleotide species. Such single stranded oligonucleotides can mediated gene expression with respect to a similar number of different genes. In one embodiment, the single stranded oligonucleotide preparation includes at least a second therapeutic agent (e.g., an agent other than an oligonucleotide).

### Route of Delivery

A composition that includes a single stranded oligonucleotide can be delivered to a subject by a variety of routes. Exemplary routes include: intravenous, intradermal, topical, rectal, parenteral, anal, intravaginal, intranasal, pulmonary, ocular, and oral. The term “therapeutically effective amount” is the amount of oligonucleotide present in the composition that is needed to provide the desired level of target gene expression in the subject to be treated to give the anticipated physiological response. The term “physiologically effective amount” is that amount delivered to a subject to give the desired palliative or curative effect. The term “pharmaceutically acceptable carrier” means that the carrier can be administered to a subject with no significant adverse toxicological effects to the subject.

The single stranded oligonucleotide molecules of the invention can be incorporated into pharmaceutical compositions suitable for administration. Such compositions typically include one or more species of single stranded oligonucleotide and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of the present invention may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, subcutaneous, intraperitoneal or intramuscular injection, or intrathecal or intraventricular administration.

The route and site of administration may be chosen to enhance targeting. For example, to target muscle cells, intramuscular injection into the muscles of interest would be a logical choice. Lung cells might be targeted by administering the single stranded oligonucleotide in aerosol form. The vascular endothelial cells could be targeted by coating a balloon catheter with the single stranded oligonucleotide and mechanically introducing the oligonucleotide.

Topical administration refers to the delivery to a subject by contacting the formulation directly to a surface of the subject. The most common form of topical delivery is to the skin, but a composition disclosed herein can also be directly applied to other surfaces of the body, e.g., to the eye, a mucous membrane, to surfaces of a body cavity or to an internal surface. As mentioned above, the most common topical delivery is to the skin. The term encompasses several routes of administration including, but not limited to, topical and transdermal. These modes of administration typically include penetration of the skin's permeability barrier and efficient delivery to the target tissue or stratum. Topical administration can be used as a means to penetrate the epidermis and dermis and ultimately achieve systemic delivery of the composition. Topical administration can also be used as a means to selectively deliver oligonucleotides to the epidermis or dermis of a subject, or to specific strata thereof, or to an underlying tissue.

Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Transdermal delivery is a valuable route for the administration of lipid soluble therapeutics. The dermis is more permeable than the epidermis and therefore absorption is much more rapid through abraded, burned or denuded skin. Inflammation and other physiologic conditions that increase blood flow to the skin also enhance transdermal adsorption. Absorption via this route may be enhanced by the use of an oily vehicle (inunction) or through the use of one or more penetration enhancers. Other effective ways to deliver a composition disclosed herein via the transdermal route include hydration of the skin and the use of controlled release topical patches. The transdermal route provides a potentially effective means to deliver a composition disclosed herein for systemic and/or local therapy. In addition, iontophoresis (transfer of ionic solutes through biological membranes under the influence of an electric field), phonophoresis or sonophoresis (use of ultrasound to enhance the absorption of various therapeutic agents across biological membranes, notably the skin and the cornea), and optimization of vehicle characteristics relative to dose position and retention at the site of administration may be useful methods for enhancing the transport of topically applied compositions across skin and mucosal sites.

Both the oral and nasal membranes offer advantages over other routes of administration. For example, oligonucleotides administered through these membranes may have a rapid onset of action, provide therapeutic plasma levels, avoid first pass effect of hepatic metabolism, and avoid exposure of the oligonucleotides to the hostile gastrointestinal (GI) environment. Additional advantages include easy access to the membrane sites so that the oligonucleotide can be applied, localized and removed easily.

In oral delivery, compositions can be targeted to a surface of the oral cavity, e.g., to sublingual mucosa which includes the membrane of ventral surface of the tongue and the floor of the mouth or the buccal mucosa which constitutes the lining of the cheek. The sublingual mucosa is relatively permeable thus giving rapid absorption and acceptable bioavailability of many agents. Further, the sublingual mucosa is convenient, acceptable and easily accessible.

A pharmaceutical composition of single stranded oligonucleotide may also be administered to the buccal cavity of a human being by spraying into the cavity, without inhalation, from a metered dose spray dispenser, a mixed micellar pharmaceutical formulation as described above and a propellant. In one embodiment, the dispenser is first shaken prior to spraying the pharmaceutical formulation and propellant into the buccal cavity.

Compositions for oral administration include powders or granules, suspensions or solutions in water, syrups, slurries, emulsions, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the nucleic acid compositions can be combined with emulsifying and suspending agents. If desired, certain sweetening and/or flavoring agents can be added.

Parenteral administration includes intravenous drip, subcutaneous, intraperitoneal or intramuscular injection, intrathecal or intraventricular administration. In some embodiments, parental administration involves administration directly to the site of disease (e.g. injection into a tumor).

Formulations for parenteral administration may include sterile aqueous solutions which may also contain buffers, diluents and other suitable additives. Intraventricular injection may be facilitated by an intraventricular catheter, for example, attached to a reservoir. For intravenous use, the total concentration of solutes should be controlled to render the preparation isotonic.

Any of the single stranded oligonucleotides described herein can be administered to ocular tissue. For example, the compositions can be applied to the surface of the eye or nearby tissue, e.g., the inside of the eyelid. For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or poly(vinyl alcohol), preservatives such as sorbic acid, EDTA or benzylchronium chloride, and the usual quantities of diluents and/or carriers. The single stranded oligonucleotide can also be administered to the interior of the eye, and can be introduced by a needle or other delivery device which can introduce it to a selected area or structure.

Pulmonary delivery compositions can be delivered by inhalation by the patient of a dispersion so that the composition, preferably single stranded oligonucleotides, within the dispersion can reach the lung where it can be readily absorbed through the alveolar region directly into blood circulation. Pulmonary delivery can be effective both for systemic delivery and for localized delivery to treat diseases of the lungs.

Pulmonary delivery can be achieved by different approaches, including the use of nebulized, aerosolized, micellular and dry powder-based formulations. Delivery can be achieved with liquid nebulizers, aerosol-based inhalers, and dry powder dispersion devices. Metered-dose devices are preferred. One of the benefits of using an atomizer or inhaler is that the potential for contamination is minimized because the devices are self-contained. Dry powder dispersion devices, for example, deliver agents that may be readily formulated as dry powders. A single stranded oligonucleotide composition may be stably stored as lyophilized or spray-dried powders by itself or in combination with suitable powder carriers. The delivery of a composition for inhalation can be mediated by a dosing timing element which can include a timer, a dose counter, time measuring device, or a time indicator which when incorporated into the device enables dose tracking, compliance monitoring, and/or dose triggering to a patient during administration of the aerosol medicament.

The term “powder” means a composition that consists of finely dispersed solid particles that are free flowing and capable of being readily dispersed in an inhalation device and subsequently inhaled by a subject so that the particles reach the lungs to permit penetration into the alveoli. Thus, the powder is said to be “respirable.” Preferably the average particle size is less than about 10 μm in diameter preferably with a relatively uniform spheroidal shape distribution. More preferably the diameter is less than about 7.5 μm and most preferably less than about 5.0 μm. Usually the particle size distribution is between about 0.1 μm and about 5 μm in diameter, particularly about 0.3 μm to about 5 μm.

The term “dry” means that the composition has a moisture content below about 10% by weight (% w) water, usually below about 5% w and preferably less it than about 3% w. A dry composition can be such that the particles are readily dispersible in an inhalation device to form an aerosol.

The types of pharmaceutical excipients that are useful as carrier include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Suitable pH adjusters or buffers include organic salts prepared from organic acids and bases, such as sodium citrate, sodium ascorbate, and the like; sodium citrate is preferred. Pulmonary administration of a micellar single stranded oligonucleotide formulation may be achieved through metered dose spray devices with propellants such as tetrafluoroethane, heptafluoroethane, dimethylfluoropropane, tetrafluoropropane, butane, isobutane, dimethyl ether and other non-CFC and CFC propellants.

Exemplary devices include devices which are introduced into the vasculature, e.g., devices inserted into the lumen of a vascular tissue, or which devices themselves form a part of the vasculature, including stents, catheters, heart valves, and other vascular devices. These devices, e.g., catheters or stents, can be placed in the vasculature of the lung, heart, or leg.

Other devices include non-vascular devices, e.g., devices implanted in the peritoneum, or in organ or glandular tissue, e.g., artificial organs. The device can release a therapeutic substance in addition to a single stranded oligonucleotide, e.g., a device can release insulin.

In one embodiment, unit doses or measured doses of a composition that includes single stranded oligonucleotide are dispensed by an implanted device. The device can include a sensor that monitors a parameter within a subject. For example, the device can include pump, e.g., and, optionally, associated electronics.

Tissue, e.g., cells or organs can be treated with a single stranded oligonucleotide, ex vivo and then administered or implanted in a subject. The tissue can be autologous, allogeneic, or xenogeneic tissue. E.g., tissue can be treated to reduce graft v. host disease. In other embodiments, the tissue is allogeneic and the tissue is treated to treat a disorder characterized by unwanted gene expression in that tissue. E.g., tissue, e.g., hematopoietic cells, e.g., bone marrow hematopoietic cells, can be treated to inhibit unwanted cell proliferation. Introduction of treated tissue, whether autologous or transplant, can be combined with other therapies. In some implementations, the single stranded oligonucleotide treated cells are insulated from other cells, e.g., by a semi-permeable porous barrier that prevents the cells from leaving the implant, but enables molecules from the body to reach the cells and molecules produced by the cells to enter the body. In one embodiment, the porous barrier is formed from alginate.

In one embodiment, a contraceptive device is coated with or contains a single stranded oligonucleotide. Exemplary devices include condoms, diaphragms, IUD (implantable uterine devices, sponges, vaginal sheaths, and birth control devices.

### Dosage

In one aspect, the invention features a method of administering a single stranded oligonucleotide (e.g., as a compound or as a component of a composition) to a subject (e.g., a human subject). In one embodiment, the unit dose is between about 10 mg and 25 mg per kg of bodyweight. In one embodiment, the unit dose is between about 1 mg and 100 mg per kg of bodyweight. In one embodiment, the unit dose is between about 0.1 mg and 500 mg per kg of bodyweight. In some embodiments, the unit dose is more than 0.001, 0.005, 0.01, 0.05, 0.1, 0.5, 1, 2, 5, 10, 25, 50 or 100 mg per kg of bodyweight.

The defined amount can be an amount effective to treat or prevent a disease or condition, e.g., a disease or condition associated with the target gene. The unit dose, for example, can be administered by injection (e.g., intravenous or intramuscular), an inhaled dose, or a topical application.

In some embodiments, the unit dose is administered daily. In some embodiments, less frequently than once a day, e.g., less than every 2, 4, 8 or 30 days. In another embodiment, the unit dose is not administered with a frequency (e.g., not a regular frequency). For example, the unit dose may be administered a single time. In some embodiments, the unit dose is administered more than once a day, e.g., once an hour, two hours, four hours, eight hours, twelve hours, etc.

In one embodiment, a subject is administered an initial dose and one or more maintenance doses of a single stranded oligonucleotide. The maintenance dose or doses are generally lower than the initial dose, e.g., one-half less of the initial dose. A maintenance regimen can include treating the subject with a dose or doses ranging from 0.0001 to 100 mg/kg of body weight per day, e.g., 100, 10, 1, 0.1, 0.01, 0.001, or 0.0001 mg per kg of bodyweight per day. The maintenance doses may be administered no more than once every 1, 5, 10, or 30 days. Further, the treatment regimen may last for a period of time which will vary depending upon the nature of the particular disease, its severity and the overall condition of the patient. In some embodiments the dosage may be delivered no more than once per day, e.g., no more than once per 24, 36, 48, or more hours, e.g., no more than once for every 5 or 8 days. Following treatment, the patient can be monitored for changes in his condition and for alleviation of the symptoms of the disease state. The dosage of the oligonucleotide may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable.

In some embodiments, the oligonucleotide pharmaceutical composition includes a plurality of single stranded oligonucleotide species. In another embodiment, the single stranded oligonucleotide species has sequences that are non-overlapping and non-adjacent to another species with respect to a naturally occurring target sequence (e.g., a lancRNA). In another embodiment, the plurality of single stranded oligonucleotide species is specific for different lancRNAs. In another embodiment, the single stranded oligonucleotide is allele specific. In some cases, a patient is treated with a single stranded oligonucleotide in conjunction with other therapeutic modalities.

Following successful treatment, it may be desirable to have the patient undergo maintenance therapy to prevent the recurrence of the disease state, wherein the compound of the invention is administered in maintenance doses, ranging from 0.0001 mg to 100 mg per kg of body weight.

The concentration of the single stranded oligonucleotide composition is an amount sufficient to be effective in treating or preventing a disorder or to regulate a physiological condition in humans. The concentration or amount of single stranded oligonucleotide administered will depend on the parameters determined for the agent and the method of administration, e.g. nasal, buccal, pulmonary. For example, nasal formulations may tend to require much lower concentrations of some ingredients in order to avoid irritation or burning of the nasal passages. It is sometimes desirable to dilute an oral formulation up to 10-100 times in order to provide a suitable nasal formulation.

Certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a single stranded oligonucleotide can include a single treatment or, preferably, can include a series of treatments. It will also be appreciated that the effective dosage of a single stranded oligonucleotide used for treatment may increase or decrease over the course of a particular treatment. For example, the subject can be monitored after administering a single stranded oligonucleotide composition. Based on information from the monitoring, an additional amount of the single stranded oligonucleotide composition can be administered.

Dosing is dependent on severity and responsiveness of the disease or condition to be treated, with the course of treatment lasting from several days to several months, or until a cure is effected or a diminution of disease state is achieved. Optimal dosing schedules can be calculated from measurements of target gene expression levels in the body of the patient. Persons of ordinary skill can easily determine optimum dosages, dosing methodologies and repetition rates. Optimum dosages may vary depending on the relative potency of individual compounds, and can generally be estimated based on EC50s found to be effective in in vitro and in vivo animal models. In some embodiments, the animal models include transgenic animals that express a human target gene. In another embodiment, the composition for testing includes a single stranded oligonucleotide that is complementary, at least in an internal region, to a sequence that is conserved between the target gene in the animal model and the target gene in a human.

In one embodiment, the administration of the single stranded oligonucleotide composition is parenteral, e.g. intravenous (e.g., as a bolus or as a diffusible infusion), intradermal, intraperitoneal, intramuscular, intrathecal, intraventricular, intracranial, subcutaneous, transmucosal, buccal, sublingual, endoscopic, rectal, oral, vaginal, topical, pulmonary, intranasal, urethral or ocular. Administration can be provided by the subject or by another person, e.g., a health care provider. The composition can be provided in measured doses or in a dispenser which delivers a metered dose. Selected modes of delivery are discussed in more detail below.

### Kits

In certain aspects of the invention, kits are provided, comprising a container housing a composition comprising a single stranded oligonucleotide. In some embodiments, the composition is a pharmaceutical composition comprising a single stranded oligonucleotide and a pharmaceutically acceptable carrier. In some embodiments, the individual components of the pharmaceutical composition may be provided in one container. Alternatively, it may be desirable to provide the components of the pharmaceutical composition separately in two or more containers, e.g., one container for single stranded oligonucleotides, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

The invention is further described in the following examples, which do not limit the scope of the invention described in the claims.

### Example 1. APOA1 and FXN lancRNA-Targeting Oligonucleotides

### Oligo Design

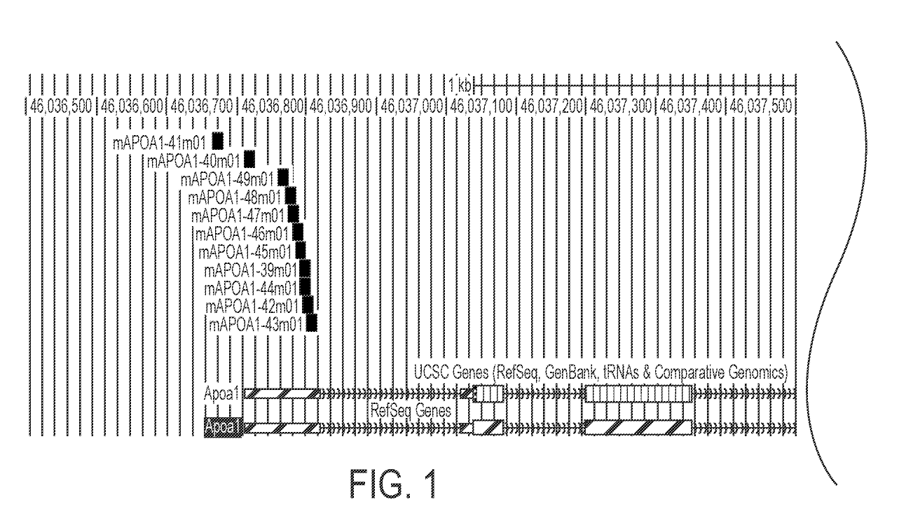

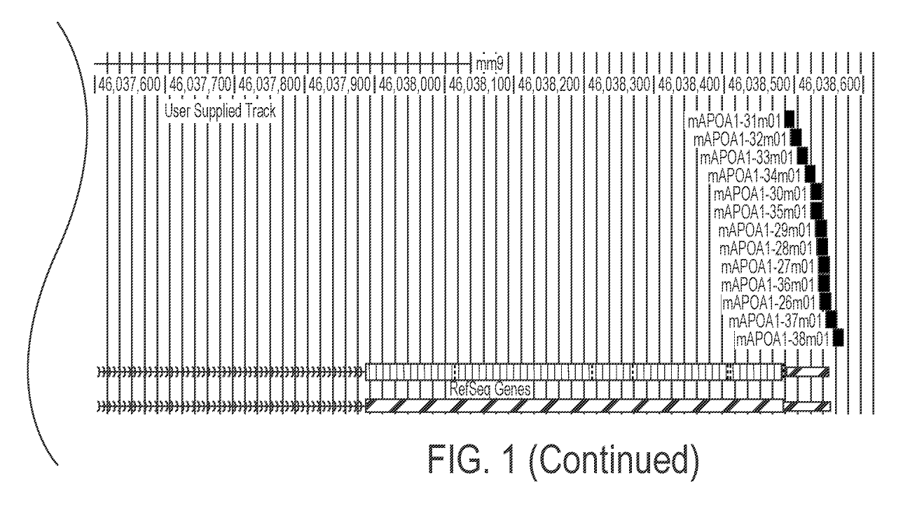

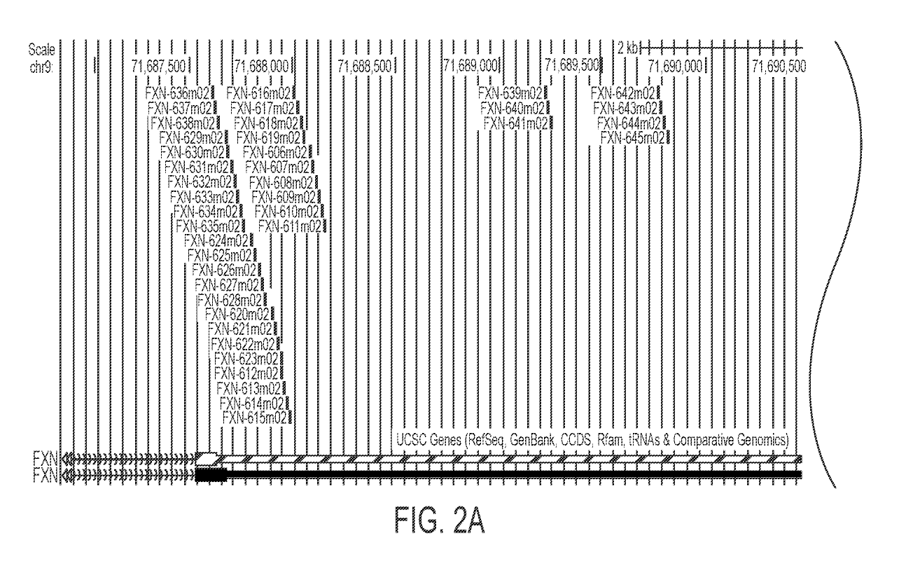

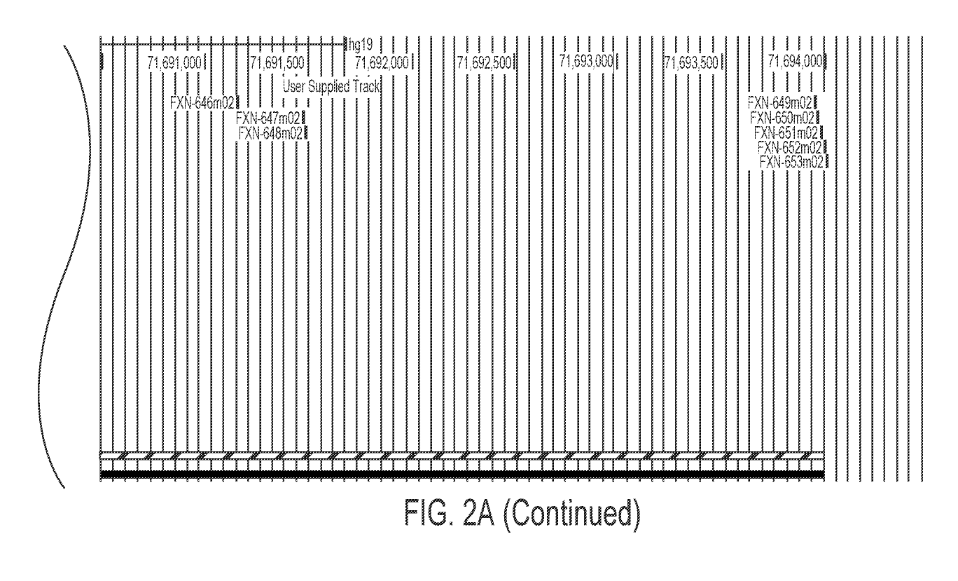

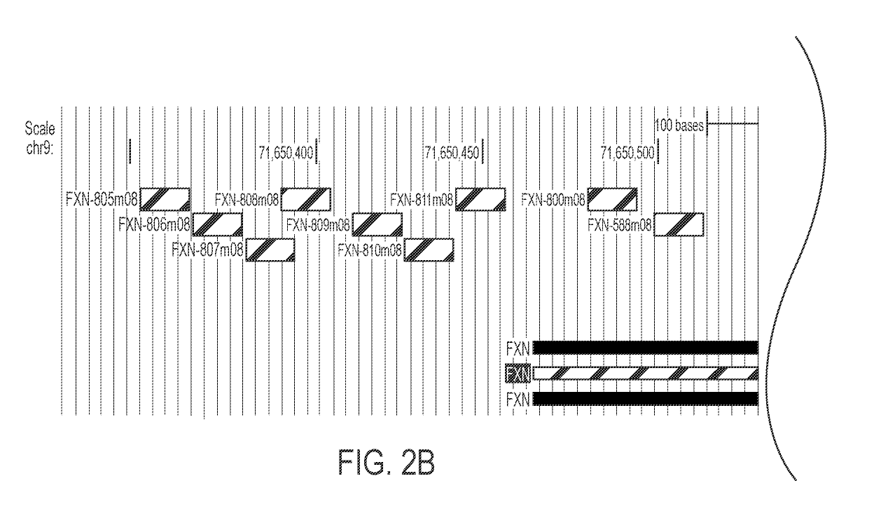

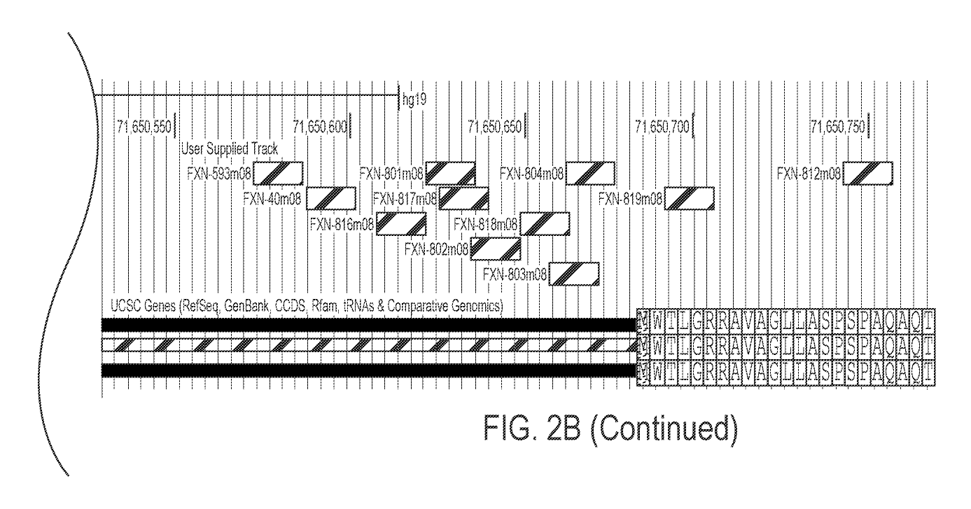

Oligonucleotides were designed to target sense and antisense regions located within a 500 nucleotide window of the transcription start and end sites of APOA1 and FXN. The oligonucleotide sequence and modification (“formatted”) patterns are provided in Table 3 below. Table 4 provides a description of the nucleotide analogs, modifications and intranucleotide linkages used for certain oligonucleotides tested and described in Table 3. A map of each gene showing where each oligonucleotide binds is provided in FIGS. 1 and 2.

TABLE 3
Oligonucleotides test

| Oligo | | Gene | SEQ | | |
|---|---|---|---|---|---|
| Name | Organism | Name | ID NO | Base Sequence | Formatted Sequence |
| FXN- | human | FXN | 1 | CGCAGTAGCCGGCCT | InaCs;omeGs;InaCs;omeAs;InaGs;omeU |
| 62 | | | | | s;InaAs;omeGs;InaCs;omeCs;InaGs;ome |
| m01 | | | | | Gs;InaCs;omeCs;InaT-Sup |
| FXN- | human | FXN | 2 | CCTGGCGTCACCCAG | InaCs;omeCs;InaTs;omeGs;InaGs;omeC |
| 601 | | | | | s;InaGs;omeUs;InaCs;omeAs;InaCs;ome |
| m01 | | | | | Cs;InaCs;omeAs;InaG-Sup |
| FXN- | human | FXN | 3 | CCCAGCCCAGGCCCA | InaCs;omeCs;InaCs;omeAs;InaGs;omeC |
| 602 | | | | | s;InaCs;omeCs;InaAs;omeGs;InaGs;ome |
| m01 | | | | | Cs;InaCs;omeCs;InaA-Sup |
| FXN- | human | FXN | 4 | CCCAGACCCTCACCC | InaCs;omeCs;InaCs;omeAs;InaGs;omeA |
| 603 | | | | | s;InaCs;omeCs;InaCs;omeUs;InaCs;ome |
| m01 | | | | | As;InaCs;omeCs;InaC-Sup |
| FXN- | human | FXN | 5 | TCCCGCGGCCGGCAG | InaTs;omeCs;InaCs;omeCs;InaGs;omeCs |
| 604 | | | | | ;InaGs;omeGs;InaCs;omeCs;InaGs;ome |
| m01 | | | | | Gs;InaCs;omeAs;InaG-Sup |
| FXN- | human | FXN | 6 | AGAGTTGGCCCCACT | InaAs;omeGs;InaAs;omeGs;InaTs;omeU |
| 605 | | | | | s;InaGs;omeGs;InaCs;omeCs;InaCs;ome |
| m01 | | | | | Cs;InaAs;omeCs;InaT-Sup |
| FXN- | human | FXN | 7 | AGCAGCTGGTCAACC | dAs;InaGs;dCs;InaAs;dGs;InaCs;dTs;Ina |
| 606 | | | | | Gs;dGs;InaTs;dCs;InaAs;dAs;InaCs;dC- |
| m02 | | | | | Sup |
| FXN- | human | FXN | 8 | TGCTCACTGTTCTAT | dTs;InaGs;dCs;InaTs;dCs;InaAs;dCs;InaT |
| 607 | | | | | s;dGs;InaTs;dTs;InaCs;dTs;InaAs;dT-Sup |
| m02 | | | | | |
| FXN- | human | FXN | 9 | CTCCAAATGAGACAC | dCs;InaTs;dCs;InaCs;dAs;InaAs;dAs;InaT |
| 608 | | | | | s;dGs;InaAs;dGs;InaAs;dCs;InaAs;dC- |
| m02 | | | | | Sup |
| FXN- | human | FXN | 10 | ATTAAAGGGTAGCCT | dAs;InaTs;dTs;InaAs;dAs;InaAs;dGs;Ina |
| 609 | | | | | Gs;dGs;InaTs;dAs;InaGs;dCs;InaCs;dT- |
| m02 | | | | | Sup |
| FXN- | human | FXN | 11 | ACAAATGTTTTCAGG | dAs;InaCs;dAs;InaAs;dAs;InaTs;dGs;InaT |
| 610 | | | | | s;dTs;InaTs;dTs;InaCs;dAs;InaGs;dG-Sup |
| m02 | | | | | |
| FXN- | human | FXN | 12 | CTTCTTTCAAAGTGT | dCs;InaTs;dTs;InaCs;dTs;InaTs;dTs;InaCs |
| 611 | | | | | ;dAs;InaAs;dAs;InaGs;dTs;InaGs;dT-Sup |
| m02 | | | | | |
| FXN- | human | FXN | 13 | TAATGTCTTATGCCT | dTs;InaAs;dAs;InaTs;dGs;InaTs;dCs;InaT |
| 612 | | | | | s;dTs;InaAs;dTs;InaGs;dCs;InaCs;dT-Sup |
| m02 | | | | | |
| FXN- | human | FXN | 14 | ATACCTGAATATAAC | dAs;InaTs;dAs;InaCs;dCs;InaTs;dGs;InaA |
| 613 | | | | | s;dAs;InaTs;dAs;InaTs;dAs;InaAs;dC-Sup |
| m02 | | | | | |
| FXN- | human | FXN | 15 | AACCTTTAAAAAAGC | dAs;InaAs;dCs;InaCs;dTs;InaTs;dTs;InaA |
| 614 | | | | | s;dAs;InaAs;dAs;InaAs;dAs;InaGs;dC- |
| m02 | | | | | Sup |
| FXN- | human | FXN | 16 | AAAATAATAAGAAGG | dAs;InaAs;dAs;InaAs;dTs;InaAs;dAs;InaT |
| 615 | | | | | s;dAs;InaAs;dGs;InaAs;dAs;InaGs;dG- |
| m02 | | | | | Sup |
| FXN- | human | FXN | 17 | AAAAATTCCAGGAGG | dAs;InaAs;dAs;InaAs;dAs;InaTs;dTs;InaC |
| 616 | | | | | s;dCs;InaAs;dGs;InaGs;dAs;InaGs;dG- |
| m02 | | | | | Sup |
| FXN- | human | FXN | 18 | GAAAATGAATTGTCT | dGs;InaAs;dAs;InaAs;dAs;InaTs;dGs;Ina |
| 617 | | | | | As;dAs;InaTs;dTs;InaGs;dTs;InaCs;dT- |
| m02 | | | | | Sup |
| FXN- | human | FXN | 19 | TCACTCTTCATTCTT | dTs;InaCs;dAs;InaCs;dTs;InaCs;dTs;InaT |
| 618 | | | | | s;dCs;InaAs;dTs;InaTs;dCs;InaTs;dT-Sup |
| m02 | | | | | |
| FXN- | human | FXN | 20 | TGAAGGATTTACTGC | dTs;InaGs;dAs;InaAs;dGs;InaGs;dAs;Ina |
| 619 | | | | | Ts;dTs;InaTs;dAs;InaCs;dTs;InaGs;dC- |
| m02 | | | | | Sup |
| FXN- | human | FXN | 21 | TTGGATTGTCGGAT | dTs;InaTs;dGs;InaGs;dAs;InaTs;dTs;InaG |
| 620 | | | | | s;dTs;InaCs;dGs;InaGs;dAs;InaT-Sup |
| m02 | | | | | |
| FXN- | human | FXN | 22 | TTCCTCCCTCACATG | dTs;InaTs;dCs;InaCs;dTs;InaCs;dCs;InaC |
| 621 | | | | | s;dTs;InaCs;dAs;InaCs;dAs;InaTs;dG-Sup |
| m02 | | | | | |
| FXN- | human | FXN | 23 | ATACCCCTTATCTTT | dAs;InaTs;dAs;InaCs;dCs;InaCs;dCs;InaT |
| 622 | | | | | s;dTs;InaAs;dTs;InaCs;dTs;InaTs;dT-Sup |
| m02 | | | | | |
| FXN- | human | FXN | 24 | TATAATGTCTTATGC | dTs;InaAs;dTs;InaAs;dAs;InaTs;dGs;InaT |
| 623 | | | | | s;dCs;InaTs;dTs;InaAs;dTs;InaGs;dC-Sup |
| m02 | | | | | |
| FXN- | human | FXN | 25 | TGAGGACAGTTGGGC | dTs;InaGs;dAs;InaGs;dGs;InaAs;dCs;Ina |
| 624 | | | | | As;dGs;InaTs;dTs;InaGs;dGs;InaGs;dC- |
| m02 | | | | | Sup |
| FXN- | human | FXN | 26 | TATGTGTCACAGCTC | dTs;InaAs;dTs;InaGs;dTs;InaGs;dTs;InaC |
| 625 | | | | | s;dAs;InaCs;dAs;InaGs;dCs;InaTs;dC-Sup |
| m02 | | | | | |
| FXN- | human | FXN | 27 | TGTAGAAAGAATGTG | dTs;InaGs;dTs;InaAs;dGs;InaAs;dAs;Ina |
| 626 | | | | | As;dGs;InaAs;dAs;InaTs;dGs;InaTs;dG- |
| m02 | | | | | Sup |
| FXN- | human | FXN | 28 | TTGCCTCCTACCTTG | dTs;InaTs;dGs;InaCs;dCs;InaTs;dCs;InaC |
| 627 | | | | | s;dTs;InaAs;dCs;InaCs;dTs;InaTs;dG-Sup |
| m02 | | | | | |
| FXN- | human | FXN | 29 | CCCCCAAGTTCTGAT | dCs;InaCs;dCs;InaCs;dCs;InaAs;dAs;InaG |
| 628 | | | | | s;dTs;InaTs;dCs;InaTs;dGs;InaAs;dT-Sup |
| m02 | | | | | |
| FXN- | human | FXN | 30 | ATGCTTGATGCCCAG | dAs;InaTs;dGs;InaCs;dTs;InaTs;dGs;InaA |
| 629 | | | | | s;dTs;InaGs;dCs;InaCs;dCs;InaAs;dG- |
| m02 | | | | | Sup |
| FXN- | human | FXN | 31 | CCCCGTTTTAAGGAC | dCs;InaCs;dCs;InaCs;dGs;InaTs;dTs;InaT |
| 630 | | | | | s;dTs;InaAs;dAs;InaGs;dGs;InaAs;dC- |
| m02 | | | | | Sup |
| FXN- | human | FXN | 32 | ATTAAAAGCTATCAG | dAs;InaTs;dTs;InaAs;dAs;InaAs;dAs;InaG |
| 631 | | | | | s;dCs;InaTs;dAs;InaTs;dCs;InaAs;dG-Sup |
| m02 | | | | | |
| FXN- | human | FXN | 33 | GCCAAGACCCCAGCT | dGs;InaCs;dCs;InaAs;dAs;InaGs;dAs;Ina |
| 632 | | | | | Cs;dCs;InaCs;dCs;InaAs;dGs;InaCs;dT- |
| m02 | | | | | Sup |
| FXN- | human | FXN | 34 | TCATTATGCAGCTGA | dTs;InaCs;dAs;InaTs;dTs;InaAs;dTs;InaG |
| 633 | | | | | s;dCs;InaAs;dGs;InaCs;dTs;InaGs;dA- |
| m02 | | | | | Sup |
| FXN- | human | FXN | 35 | GGTCTGTTTTTTGTT | dGs;InaGs;dTs;InaCs;dTs;InaGs;dTs;InaT |
| 634 | | | | | s;dTs;InaTs;dTs;InaTs;dGs;InaTs;dT-Sup |
| m02 | | | | | |
| FXN- | human | FXN | 36 | GTTGTTGTTGTTTAT | dGs;InaTs;dTs;InaGs;dTs;InaTs;dGs;InaT |
| 635 | | | | | s;dTs;InaGs;dTs;InaTs;dTs;InaAs;dT-Sup |
| m02 | | | | | |
| FXN- | human | FXN | 37 | GCAGAGCTCACTAAA | dGs;InaCs;dAs;InaGs;dAs;InaGs;dCs;Ina |
| 636 | | | | | Ts;dCs;InaAs;dCs;InaTs;dAs;InaAs;dA- |
| m02 | | | | | Sup |
| FXN- | human | FXN | 38 | GCCTTAAAAACCAAA | dGs;InaCs;dCs;InaTs;dTs;InaAs;dAs;InaA |
| 637 | | | | | s;dAs;InaAs;dCs;InaCs;dAs;InaAs;dA- |
| m02 | | | | | Sup |
| FXN- | human | FXN | 39 | CTGGACTTGTCTTCC | dCs;InaTs;dGs;InaGs;dAs;InaCs;dTs;InaT |
| 638 | | | | | s;dGs;InaTs;dCs;InaTs;dTs;InaCs;dC-Sup |
| m02 | | | | | |
| FXN- | human | FXN | 40 | CAGGACATTAAAATT | dCs;InaAs;dGs;InaGs;dAs;InaCs;dAs;Ina |
| 640 | | | | | Ts;dTs;InaAs;dAs;InaAs;dAs;InaTs;dT- |
| m02 | | | | | Sup |
| FXN- | human | FXN | 41 | CATGCAAAGTTATGC | dCs;InaAs;dTs;InaGs;dCs;InaAs;dAs;InaA |
| 641 | | | | | s;dGs;InaTs;dTs;InaAs;dTs;InaGs;dC-Sup |
| m02 | | | | | |
| FXN- | human | FXN | 42 | AAGTGCAGTAGGCCA | dAs;InaAs;dGs;InaTs;dGs;InaCs;dAs;Ina |
| 642 | | | | | Gs;dTs;InaAs;dGs;InaGs;dCs;InaCs;dA- |
| m02 | | | | | Sup |
| FXN- | human | FXN | 43 | GTGCCAGTGAGAAAA | dGs;InaTs;dGs;InaCs;dCs;InaAs;dGs;Ina |
| 643 | | | | | Ts;dGs;InaAs;dGs;InaAs;dAs;InaAs;dA- |
| m02 | | | | | Sup |
| FXN- | human | FXN | 44 | TAAATAACATCATAC | dTs;InaAs;dAs;InaAs;dTs;InaAs;dAs;InaC |
| 644 | | | | | s;dAs;InaTs;dCs;InaAs;dTs;InaAs;dC-Sup |
| m02 | | | | | |
| FXN- | human | FXN | 45 | ATGTTTGTATGTGTT | dAs;InaTs;dGs;InaTs;dTs;InaTs;dGs;InaT |
| 645 | | | | | s;dAs;InaTs;dGs;InaTs;dGs;InaTs;dT-Sup |
| m02 | | | | | |
| FXN- | human | FXN | 46 | AATCTATAAAATGGA | dAs;InaAs;dTs;InaCs;dTs;InaAs;dTs;InaA |
| 646 | | | | | s;dAs;InaAs;dAs;InaTs;dGs;InaGs;dA- |
| m02 | | | | | Sup |
| FXN- | human | FXN | 47 | CAGTTTGCATTAAAT | dCs;InaAs;dGs;InaTs;dTs;InaTs;dGs;InaC |
| 647 | | | | | s;dAs;InaTs;dTs;InaAs;dAs;InaAs;dT-Sup |
| m02 | | | | | |
| FXN- | human | FXN | 48 | TATAGGTTTACAATA | dTs;InaAs;dTs;InaAs;dGs;InaGs;dTs;InaT |
| 648 | | | | | s;dTs;InaAs;dCs;InaAs;dAs;InaTs;dA-Sup |
| m02 | | | | | |
| FXN- | human | FXN | 49 | GTTATAATTATATGT | dGs;InaTs;dTs;InaAs;dTs;InaAs;dAs;InaT |
| 649 | | | | | s;dTs;InaAs;dTs;InaAs;dTs;InaGs;dT-Sup |
| m02 | | | | | |
| FXN- | human | FXN | 50 | TTAAGATAGTTGTTC | dTs;InaTs;dAs;InaAs;dGs;InaAs;dTs;InaA |
| 650 | | | | | s;dGs;InaTs;dTs;InaGs;dTs;InaTs;dC-Sup |
| m02 | | | | | |
| FXN- | human | FXN | 51 | AATAAACTCTAAATA | dAs;InaAs;dTs;InaAs;dAs;InaAs;dCs;InaT |
| 651 | | | | | s;dCs;InaTs;dAs;InaAs;dAs;InaTs;dA-Sup |
| m02 | | | | | |
| FXN- | human | FXN | 52 | ACCCCAACTCCAAGA | dAs;InaCs;dCs;InaCs;dCs;InaAs;dAs;InaC |
| 652 | | | | | s;dTs;InaCs;dCs;InaAs;dAs;InaGs;dA-Sup |
| m02 | | | | | |
| FXN- | human | FXN | 53 | GTGTTAGCAAGAAAT | dGs;InaTs;dGs;InaTs;dTs;InaAs;dGs;InaC |
| 653 | | | | | s;dAs;InaAs;dGs;InaAs;dAs;InaAs;dT- |
| m02 | | | | | Sup |
| Apoa | mouse | Apoa | 54 | TCCAAAATGGAATA | InaTs;omeCs;InaCs;omeAs;InaAs;om |
| 1_mu | | 1 | | G | eAs;InaAs;omeTs;InaGs;omeGs;InaA |
| s-26 | | | | | s;omeAs;InaTs;omeAs;InaG-Sup |
| m01 | | | | | |
| Apoa | mouse | Apoa | 55 | TTTCCAAAATGGAA | InaTs;omeTs;InaTs;omeCs;InaCs;om |
| 1_mu | | 1 | | T | eAs;InaAs;omeAs;InaAs;omeTs;InaG |
| s-27 | | | | | s;omeGs;InaAs;omeAs;InaT-Sup |
| m01 | | | | | |
| Apoa | mouse | Apoa | 56 | ACCTTTCCAAAATG | InaAs;omeCs;InaCs;omeTs;InaTs;om |
| 1_mu | | 1 | | G | eTs;InaCs;omeCs;InaAs;omeAs;InaA |
| s-28 | | | | | s;omeAs;InaTs;omeGs;InaG-Sup |
| m01 | | | | | |
| Apoa | mouse | Apoa | 57 | AACCTTTCCAAAAT | InaAs;omeAs;InaCs;omeCs;InaTs;om |
| 1_mu | | 1 | | G | eTs;InaTs;omeCs;InaCs;omeAs;InaA |
| s-29 | | | | | s;omeAs;InaAs;omeTs;InaG-Sup |
| m01 | | | | | |
| Apoa | mouse | Apoa | 58 | ACAATAAACCTTTC | InaAs;omeCs;InaAs;omeAs;InaTs;o |
| 1_mu | | 1 | | C | meAs;InaAs;omeAs;InaCs;omeCs;In |
| s-30 | | | | | aTs;omeTs;InaTs;omeCs;InaC-Sup |
| m01 | | | | | |
| Apoa | mouse | Apoa | 59 | GGTGCCCGCTTCCA | InaGs;omeGs;InaTs;omeGs;InaCs;o |
| 1_mu | | 1 | | C | meCs;InaCs;omeGs;InaCs;omeTs;Ina |
| s-31 | | | | | Ts;omeCs;InaCs;omeAs;InaC-Sup |
| m01 | | | | | |
| Apoa | mouse | Apoa | 60 | TCCACTCCCCACCC | InaTs;omeCs;InaCs;omeAs;InaCs;om |
| 1_mu | | 1 | | C | eTs;InaCs;omeCs;InaCs;omeCs;InaA |
| s-32 | | | | | s;omeCs;InaCs;omeCs;InaC-Sup |
| m01 | | | | | |
| Apoa | mouse | Apoa | 61 | ACCCCCGCATTGGC | InaAs;omeCs;InaCs;omeCs;InaCs;om |
| 1_mu | | 1 | | T | eCs;InaGs;omeCs;InaAs;omeTs;InaT |
| s-33 | | | | | s;omeGs;InaGs;omeCs;InaT-Sup |
| m01 | | | | | |
| Apoa | mouse | Apoa | 62 | TGGCTTTCTTACAA | InaTs;omeGs;InaGs;omeCs;InaTs;o |
| 1_mu | | 1 | | T | meTs;InaTs;omeCs;InaTs;omeTs;Ina |
| s-34 | | | | | As;omeCs;InaAs;omeAs;InaT-Sup |
| m01 | | | | | |
| Apoa | mouse | Apoa | 63 | GGAATAGCTTCTTT | InaGs;omeGs;InaAs;omeAs;InaTs;o |
| 1_mu | | 1 | | C | meAs;InaGs;omeCs;InaTs;omeTs;Ina |
| s-35 | | | | | Cs;omeTs;InaTs;omeTs;InaC-Sup |
| m01 | | | | | |
| Apoa | mouse | Apoa | 64 | CTTTCTTTGGGGGA | InaCs;omeTs;InaTs;omeTs;InaCs;om |
| 1_mu | | | | C | eTs;InaTs;omeTs;InaGs;omeGs;InaG |
| s-36 | | 1 | | | s;omeGs;InaGs;omeAs;InaC-Sup |
| m01 | | | | | |
| Apoa | mouse | Apoa | 65 | CACCCAGACTGTCG | InaCs;omeAs;InaCs;omeCs;InaCs;om |
| 1_mu | | 1 | | G | eAs;InaGs;omeAs;InaCs;omeTs;InaG |
| s-37 | | | | | s;omeTs;InaCs;omeGs;InaG-Sup |
| m01 | | | | | |
| Apoa | mouse | Apoa | 66 | CAGGGCCAGGCTG | InaCs;omeAs;InaGs;omeGs;InaGs;o |
| 1_mu | | 1 | | AG | meCs;InaCs;omeAs;InaGs;omeGs;In |
| s-38 | | | | | aCs;omeTs;InaGs;omeAs;InaG-Sup |
| m01 | | | | | |
| Apoa | mouse | Apoa | 67 | GCTGATCCTTGAAC | InaGs;omeCs;InaTs;omeGs;InaAs;o |
| 1_mu | | 1 | | T | meTs;InaCs;omeCs;InaTs;omeTs;Ina |
| s-39 | | | | | Gs;omeAs;InaAs;omeCs;InaT-Sup |
| m01 | | | | | |
| Apoa | mouse | Apoa | 68 | AGACTGTCGGAGA | InaAs;omeGs;InaAs;omeCs;InaTs;o |
| 1_mu | | 1 | | GC | meGs;InaTs;omeCs;InaGs;omeGs;In |
| s-40 | | | | | aAs;omeGs;InaAs;omeGs;InaC-Sup |
| m01 | | | | | |
| Apoa | mouse | Apoa | 69 | TGTCGGAGAGCTC | InaTs;omeGs;InaTs;omeCs;InaGs;o |
| 1_mu | | 1 | | CG | meGs;InaAs;omeGs;InaAs;omeGs;In |
| s-41 | | | | | aCs;omeTs;InaCs;omeCs;InaG-Sup |
| m01 | | | | | |
| Apoa | mouse | Apoa | 70 | GCTGGACACCCAG | InaGs;omeCs;InaTs;omeGs;InaGs;o |
| 1_mu | | 1 | | AC | meAs;InaCs;omeAs;InaCs;omeCs;In |
| s-42 | | | | | aCs;omeAs;InaGs;omeAs;InaC-Sup |
| m01 | | | | | |
| Apoa | mouse | Apoa | 71 | GAAGAGCTGGACA | InaGs;omeAs;InaAs;omeGs;InaAs;o |
| 1_mu | | 1 | | CC | meGs;InaCs;omeTs;InaGs;omeGs;In |
| s-43 | | | | | aAs;omeCs;InaAs;omeCs;InaC-Sup |
| m01 | | | | | |
| Apoa | mouse | Apoa | 72 | GGGAAGAAGAGCT | InaGs;omeGs;InaGs;omeAs;InaAs;o |
| 1_mu | | 1 | | GG | meGs;InaAs;omeAs;InaGs;omeAs;In |
| s-44 | | | | | aGs;omeCs;InaTs;omeGs;InaG-Sup |
| m01 | | | | | |
| Apoa | mouse | Apoa | 73 | GACCAGGGAAGAA | InaGs;omeAs;InaCs;omeCs;InaAs;o |
| 1_mu | | 1 | | GA | meGs;InaGs;omeGs;InaAs;omeAs;In |
| s-45 | | | | | aGs;omeAs;InaAs;omeGs;InaA-Sup |
| m01 | | | | | |
| Apoa | mouse | Apoa | 74 | CACATATATAGACC | InaCs;omeAs;InaCs;omeAs;InaTs;om |
| 1_mu | | 1 | | A | eAs;InaTs;omeAs;InaTs;omeAs;InaG |
| s-46 | | | | | s;omeAs;InaCs;omeCs;InaA-Sup |
| m01 | | | | | |
| FXN- | human | FXN | 75 | GTCTCCCTTGGGTC | InaGs;omeUs;InaCs;omeUs;InaCs;o |
| 800 | | | | A | meCs;InaCs;omeUs;InaTs;omeGs;In |
| m01 | | | | | aGs;omeGs;InaTs;omeCs;InaA-Sup |
| FXN- | human | FXN | 76 | TGCGGCCAGTGGC | InaTs;omeGs;InaCs;omeGs;InaGs;o |
| 801 | | | | CA | meCs;InaCs;omeAs;InaGs;omeUs;In |
| m01 | | | | | aGs;omeGs;InaCs;omeCs;InaA-Sup |
| FXN- | human | FXN | 77 | CACCAGGGGTCGC | InaCs;omeAs;InaCs;omeCs;InaAs;o |
| 802 | | | | CG | meGs;deazaGs;omeGs;InaGs;omeU |
| m01 | | | | | s;InaCs;omeGs;InaCs;omeCs;InaG- |
| | | | | | Sup |
| FXN- | human | FXN | 78 | CAGCGCTGGAGGG | InaCs;omeAs;InaGs;omeCs;InaGs;o |
| 803 | | | | CG | meCs;InaTs;omeGs;InaGs;omeAs;In |
| m01 | | | | | aGs;omeGs;deazaGs;omeCs;InaG- |
| | | | | | Sup |
| FXN- | human | FXN | 79 | CTGGAGGGCGGAG | InaCs;omeUs;InaGs;omeGs;InaAs;o |
| 804 | | | | CG | meGs;deazaGs;omeGs;InaCs;omeGs |
| m01 | | | | | ;InaGs;omeAs;InaGs;omeCs;InaG- |
| | | | | | Sup |
| FXN- | human | FXN | 80 | GTCTCCCTTGGGTC | InaGs;InaTs;InaCs;dTs;dCs;dCs;dCs;d |
| 800 | | | | A | Ts;dTs;dGs;dGs;dGs;InaTs;InaCs;Ina |
| m08 | | | | | A-Sup |
| FXN- | human | FXN | 81 | TGCGGCCAGTGGC | InaTs;InaGs;InaCs;dGs;dGs;dCs;dCs; |
| 801 | | | | CA | dAs;dGs;dTs;dGs;dGs;InaCs;InaCs;In |
| m08 | | | | | aA-Sup |
| FXN- | human | FXN | 82 | CACCAGGGGTCGC | InaCs;InaAs;InaCs;dCs;dAs;dGs;dGs; |
| 802 | | | | CG | dGs;dGs;dTs;dCs;dGs;InaCs;InaCs;In |
| m08 | | | | | aG-Sup |
| FXN- | human | FXN | 83 | CAGCGCTGGAGGG | InaCs;InaAs;InaGs;dCs;dGs;dCs;dTs; |
| 803 | | | | CG | dGs;dGs;dAs;dGs;dGs;InaGs;InaCs;1 |
| m08 | | | | | naG-Sup |
| FXN- | human | FXN | 84 | CTGGAGGGCGGAG | InaCs;InaTs;InaGs;dGs;dAs;dGs;dGs; |
| 804 | | | | CG | dGs;dCs;dGs;dGs;dAs;InaGs;InaCs;In |
| m08 | | | | | aG-Sup |
| FXN- | human | FXN | 85 | AACTGCTGTAAACC | InaAs;InaAs;InaCs;dTs;dGs;dCs;dTs; |
| 805 | | | | C | dGs;dTs;dAs;dAs;dAs;InaCs;InaCs;In |
| m08 | | | | | aC-Sup |
| FXN- | human | FXN | 86 | ATACCGGCGGCCA | InaAs;InaTs;InaAs;dCs;dCs;dGs;dGs; |
| 806 | | | | AG | dCs;dGs;dGs;dCs;dCs;InaAs;InaAs;In |
| m08 | | | | | aG-Sup |
| FXN- | human | FXN | 87 | CAGCCTCAATTTGT | InaCs;InaAs;InaGs;dCs;dCs;dTs;dCs; |
| 807 | | | | G | dAs;dAs;dTs;dTs;dTs;InaGs;InaTs;Ina |
| m08 | | | | | G-Sup |
| FXN- | human | FXN | 88 | CATGCACCCACTTC | InaCs;InaAs;InaTs;dGs;dCs;dAs;dCs; |
| 808 | | | | C | dCs;dCs;dAs;dCs;dTs;InaTs;InaCs;Ina |
| m08 | | | | | C-Sup |
| FXN- | human | FXN | 89 | CAGCAAGACAGCA | InaCs;InaAs;InaGs;dCs;dAs;dAs;dGs; |
| 809 | | | | GC | dAs;dCs;dAs;dGs;dCs;InaAs;InaGs;In |
| m08 | | | | | aC-Sup |
| FXN- | human | FXN | 90 | TCCCAAGTTCCTCC | InaTs;InaCs;InaCs;dCs;dAs;dAs;dGs; |
| 810 | | | | T | dTs;dTs;dCs;dCs;dTs;InaCs;InaCs;Ina |
| m08 | | | | | T-Sup |
| FXN- | human | FXN | 91 | GTTTAGAATTTTAG | InaGs;InaTs;InaTs;dTs;dAs;dGs;dAs; |
| 811 | | | | A | dAs;dTs;dTs;dTs;dTs;InaAs;InaGs;Ina |
| m08 | | | | | A-Sup |
| FXN- | human | FXN | 92 | GGCTGCAGTCTCCC | InaGs;InaGs;InaCs;dTs;dGs;dCs;dAs; |
| 812 | | | | T | dGs;dTs;dCs;dTs;dCs;InaCs;InaCs;Ina |
| m08 | | | | | T-Sup |
| FXN- | human | FXN | 93 | TCCTGGTTGCACTC | InaTs;InaCs;InaCs;dTs;dGs;dGs;dTs; |
| 588 | | | | C | dTs;dGs;dCs;dAs;dCs;InaTs;InaCs;In |
| m08 | | | | | aC-Sup |
| FXN- | human | FXN | 94 | AGTTCTTCCTGAGG | InaAs;InaGs;InaTs;dTs;dCs;dTs;dTs;d |
| 593 | | | | T | Cs;dCs;dTs;dGs;dAs;InaGs;InaGs;Ina |
| m08 | | | | | T-Sup |
| FXN- | human | FXN | 95 | CTAACCTCTAGCTG | InaCs;InaTs;InaAs;dAs;dCs;dCs;dTs;d |
| 40 | | | | C | Cs;dTs;dAs;dGs;dCs;InaTs;InaGs;Ina |
| m08 | | | | | C-Sup |
| FXN- | human | FXN | 96 | CACAGAAGAGTGC | InaCs;InaAs;InaCs;dAs;dGs;dAs;dAs; |
| 816 | | | | CT | dGs;dAs;dGs;dTs;dGs;InaCs;InaCs;In |
| m08 | | | | | aT-Sup |
| FXN- | human | FXN | 97 | GCCAGTGGCCACC | InaGs;InaCs;InaCs;dAs;dGs;dTs;dGs; |
| 817 | | | | AG | dGs;dCs;dCs;dAs;dCs;InaCs;InaAs;In |
| m08 | | | | | aG-Sup |
| FXN- | human | FXN | 98 | GCAGCACCCAGCG | InaGs;InaCs;InaAs;dGs;dCs;dAs;dCs; |
| 818 | | | | CT | dCs;dCs;dAs;dGs;dCs;InaGs;InaCs;In |
| m08 | | | | | aT-Sup |
| FXN- | human | FXN | 99 | GAGCAGCATGTGG | InaGs;InaAs;InaGs;dCs;dAs;dGs;dCs; |
| 819 | | | | AC | dAs;dTs;dGs;dTs;dGs;InaGs;InaAs;In |
| m08 | | | | | aC-Sup |
| FXN- | human | FXN | 100 | TCTCCCACTCAACA | InaTs;InaCs;InaTs;dCs;dCs;dCs;dAs;d |
| 820 | | | | C | Cs;dTs;dCs;dAs;dAs;InaCs;InaAs;Ina |
| m08 | | | | | C-Sup |
| FXN- | human | FXN | 101 | TCACACCTGTTAGT | InaTs;InaCs;InaAs;dCs;dAs;dCs;dCs; |
| 821 | | | | T | dTs;dGs;dTs;dTs;dAs;InaGs;InaTs;In |
| m08 | | | | | aT-Sup |
| FXN- | human | FXN | 102 | TTCCTCTTGACACTT | InaTs;InaTs;InaCs;dCs;dTs;dCs;dTs;d |
| 822 | | | | | Ts;dGs;dAs;dCs;dAs;InaCs;InaTs;Ina |
| m08 | | | | | T-Sup |
| FXN- | human | FXN | 103 | GTCATTTAGCATCC | InaGs;InaTs;InaCs;dAs;dTs;dTs;dTs;d |
| 823 | | | | T | As;dGs;dCs;dAs;dTs;InaCs;InaCs;Ina |
| m08 | | | | | T-Sup |
| FXN- | human | FXN | 104 | AAGTATGTAAACAT | InaAs;InaAs;InaGs;dTs;dAs;dTs;dGs; |
| 824 | | | | G | dTs;dAs;dAs;dAs;dCs;InaAs;InaTs;In |
| m08 | | | | | aG-Sup |
| FXN- | human | FXN | 105 | CACGATTCACAAAG | InaCs;InaAs;InaCs;dGs;dAs;dTs;dTs; |
| 825 | | | | T | dCs;dAs;dCs;dAs;dAs;InaAs;InaGs;In |
| m08 | | | | | aT-Sup |
| FXN- | human | FXN | 106 | GGCTTTGGAAGAA | InaGs;InaGs;InaCs;dTs;dTs;dTs;dGs; |
| 826 | | | | CT | dGs;dAs;dAs;dGs;dAs;InaAs;InaCs;In |
| m08 | | | | | aT-Sup |
| FXN- | human | FXN | 107 | TTAGTACCTTCCCA | InaTs;InaTs;InaAs;dGs;dTs;dAs;dCs; |
| 827 | | | | T | dCs;dTs;dTs;dCs;dCs;InaCs;InaAs;Ina |
| m08 | | | | | T-Sup |
| FXN- | human | FXN | 108 | GTCTCCCTTGGGTC | InaGs;omeUs;InaCs;omeUs;InaCs;o |
| 800 | | | | A | meCs;InaCs;omeUs;InaTs;omeGs;In |
| m01 | | | | | aGs;omeGs;InaTs;omeCs;InaA-Sup |
| FXN- | human | FXN | 109 | CGCTCCGCCCTCCAG | dCs;InaGs;dCs;InaTs;dCs;InaCs;dGs;InaC |
| 375 | | | | | s;dCs;InaCs;dTs;InaCs;dCs;InaAs;dG-Sup |
| m02 | | | | | |
| FXN- | human | FXN | 110 | ATTATTTTGCTTTTT | dAs;InaTs;dTs;InaAs;dTs;InaTs;dTs;InaTs |
| 390 | | | | | ;dGs;InaCs;dTs;InaTs;dTs;InaTs;dT-Sup |
| m02 | | | | | |
| FXN- | human | FXN | 111 | AGGCCACGGCGGCC | InaAs;omeGs;InaGs;omeCs;InaCs;omeA |
| 577 | | | | GCA | s;InaCs;omeGs;InaGs;omeCs;InaGs;ome |
| m01 | | | | | Gs;InaCs;omeCs;InaGs;omeCs;InaA-Sup |
| FXN- | human | FXN | 112 | CATCGATGTCGGTGC | InaCs;omeAs;InaTs;omeCs;InaGs;omeA |
| 578 | | | | GC | s;InaTs;omeGs;InaTs;omeCs;InaGs;ome |
| m01 | | | | | Gs;InaTs;omeGs;InaCs;omeGs;InaC-Sup |
| FXN- | human | FXN | 113 | ACACATAGCCCAACT | InaAs;InaCs;InaAs;dCs;dAs;dTs;dAs;dGs; |
| 695 | | | | | dCs;dCs;dCs;dAs;InaAs;InaCs;InaT-Sup |
| m08 | | | | | |
| HAMP- | human | HAMP | 114 | CTCAGACCACCGCC | InaCs;omeUs;InaCs;omeAs;InaGs;o |
| 17 | | | | T | meAs;InaCs;omeCs;InaAs;omeCs;In |
| m01 | | | | | aCs;omeGs;InaCs;omeCs;InaT-Sup |
| HAMP- | human | HAMP | 115 | CCACCGCCTCCCCT | InaCs;omeCs;InaAs;omeCs;InaCs;om |
| 18 | | | | G | eGs;InaCs;omeCs;InaTs;omeCs;InaC |
| m01 | | | | | s;omeCs;InaCs;omeUs;InaG-Sup |
| HAMP- | human | HAMP | 116 | CAGGCCCCATAAAA | InaCs;omeAs;InaGs;omeGs;InaCs;o |
| 19 | | | | G | meCs;InaCs;omeCs;InaAs;omeUs;In |
| m01 | | | | | aAs;omeAs;InaAs;omeAs;InaG-Sup |
| HAMP- | human | HAMP | 117 | CATAAAAGCGACT | InaCs;omeAs;InaTs;omeAs;InaAs;o |
| 20 | | | | GT | meAs;InaAs;omeGs;InaCs;omeGs;In |
| m01 | | | | | aAs;omeCs;InaTs;omeGs;InaT-Sup |
| HAMP- | human | HAMP | 118 | ACTGTCACTCGGTC | InaAs;omeCs;InaTs;omeGs;InaTs;om |
| 21 | | | | C | eCs;InaAs;omeCs;InaTs;omeCs;InaG |
| m01 | | | | | s;omeGs;InaTs;omeCs;InaC-Sup |
| HAMP- | human | HAMP | 119 | ACTCGGTCCCAGAC | InaAs;omeCs;InaTs;omeCs;InaGs;o |
| 22 | | | | A | meGs;InaTs;omeCs;InaCs;omeCs;Ina |
| m01 | | | | | As;omeGs;InaAs;omeCs;InaA-Sup |
| HAMP- | human | HAMP | 120 | AGACACCAGAGCA | InaAs;omeGs;InaAs;omeCs;InaAs;o |
| 23 | | | | AG | meCs;InaCs;omeAs;InaGs;omeAs;In |
| m01 | | | | | aGs;omeCs;InaAs;omeAs;InaG-Sup |
| HAMP- | human | HAMP | 121 | GAGCAAGCTCAAG | InaGs;omeAs;InaGs;omeCs;InaAs;o |
| 24 | | | | AC | meAs;InaGs;omeCs;InaTs;omeCs;In |
| m01 | | | | | aAs;omeAs;InaGs;omeAs;InaC-Sup |
| HAMP- | human | HAMP | 122 | GCAAGACGTAGAA | InaGs;omeCs;InaAs;omeAs;InaGs;o |
| 25 | | | | CC | meAs;InaCs;omeGs;InaTs;omeAs;In |
| m01 | | | | | aGs;omeAs;InaAs;omeCs;InaC-Sup |
| HAMP- | human | HAMP | 123 | AGACGTAGAACCT | InaAs;omeGs;InaAs;omeCs;InaGs;o |
| 26 | | | | AC | meUs;InaAs;omeGs;InaAs;omeAs;In |
| m01 | | | | | aCs;omeCs;InaTs;omeAs;InaC-Sup |
| HAMP- | human | HAMP | 124 | AGAACCTACCTGCC | InaAs;omeGs;InaAs;omeAs;InaCs;o |
| 27 | | | | C | meCs;InaTs;omeAs;InaCs;omeCs;Ina |
| m01 | | | | | Ts;omeGs;InaCs;omeCs;InaC-Sup |
| HAMP- | human | HAMP | 125 | ACATAGGTCTTGGA | InaAs;omeCs;InaAs;omeUs;InaAs;o |
| 28 | | | | A | meGs;InaGs;omeUs;InaCs;omeUs;In |
| m01 | | | | | aTs;omeGs;InaGs;omeAs;InaA-Sup |
| HAMP- | human | HAMP | 126 | AGGTCTTGGAATA | InaAs;omeGs;InaGs;omeUs;InaCs;o |
| 29 | | | | AA | meUs;InaTs;omeGs;InaGs;omeAs;In |
| m01 | | | | | aAs;omeUs;InaAs;omeAs;InaA-Sup |
| HAMP- | human | HAMP | 127 | TGGAATAAAATGG | InaTs;omeGs;InaGs;omeAs;InaAs;o |
| 30 | | | | CT | meUs;InaAs;omeAs;InaAs;omeAs;In |
| m01 | | | | | aTs;omeGs;InaGs;omeCs;InaT-Sup |
| HAMP- | human | HAMP | 128 | TGGCTGGTTCTTTT | InaTs;omeGs;InaGs;omeCs;InaTs;o |
| 31 | | | | G | meGs;InaGs;omeUs;InaTs;omeCs;In |
| m01 | | | | | aTs;omeUs;InaTs;omeUs;InaG-Sup |
| HAMP- | human | HAMP | 129 | GTTTTCCAAACCAG | InaGs;omeUs;InaTs;omeUs;InaTs;o |
| 32 | | | | A | meCs;InaCs;omeAs;InaAs;omeAs;In |
| m01 | | | | | aCs;omeCs;InaAs;omeGs;InaA-Sup |
| HAMP- | human | HAMP | 130 | ACCAGAGTGTCTGT | InaAs;omeCs;InaCs;omeAs;InaGs;o |
| 33 | | | | T | meAs;InaGs;omeUs;InaGs;omeUs;In |
| m01 | | | | | aCs;omeUs;InaGs;omeUs;InaT-Sup |
| HAMP- | human | HAMP | 131 | AGTGTCTGTTGTCC | InaAs;omeGs;InaTs;omeGs;InaTs;o |
| 34 | | | | T | meCs;InaTs;omeGs;InaTs;omeUs;In |
| m01 | | | | | aGs;omeUs;InaCs;omeCs;InaT-Sup |
| HAMP- | human | HAMP | 132 | GTTGTCCTTTCTCTC | InaGs;omeUs;InaTs;omeGs;InaTs;o |
| 35 | | | | | meCs;InaCs;omeUs;InaTs;omeUs;In |
| m01 | | | | | aCs;omeUs;InaCs;omeUs;InaC-Sup |
| HAMP- | human | HAMP | 133 | CTTTCTCTCTGCCG | InaCs;omeUs;InaTs;omeUs;InaCs;o |
| 36 | | | | A | meUs;InaCs;omeUs;InaCs;omeUs;In |
| m01 | | | | | aGs;omeCs;InaCs;omeGs;InaA-Sup |
| HAMP- | human | HAMP | 134 | CTGCCGAGTGTCTG | InaCs;omeUs;InaGs;omeCs;InaCs;o |
| 37 | | | | T | meGs;InaAs;omeGs;InaTs;omeGs;In |
| m01 | | | | | aTs;omeCs;InaTs;omeGs;InaT-Sup |
| HAMP- | human | HAMP | 135 | CTCAGACCACCGCC | InaCs;InaTs;InaCs;dAs;dGs;dAs;dCs; |
| 17 | | | | T | dCs;dAs;dCs;dCs;dGs;InaCs;InaCs;In |
| m08 | | | | | aT-Sup |
| HAMP- | human | HAMP | 136 | CCACCGCCTCCCCT | InaCs;InaCs;InaAs;dCs;dCs;dGs;dCs; |
| 18 | | | | | dCs;dTs;dCs;dCs;dCs;InaCs;InaTs;Ina |
| m08 | | | | | G-Sup |
| HAMP- | human | HAMP | 137 | CAGGCCCCATAAAA | InaCs;InaAs;InaGs;dGs;dCs;dCs;dCs; |
| 19 | | | | G | dCs;dAs;dTs;dAs;dAs;InaAs;InaAs;In |
| m08 | | | | | aG-Sup |
| HAMP- | human | HAMP | 138 | CATAAAAGCGACT | InaCs;InaAs;InaTs;dAs;dAs;dAs;dAs; |
| 20 | | | | GT | dGs;dCs;dGs;dAs;dCs;InaTs;InaGs;In |
| m08 | | | | | aT-Sup |
| HAMP- | human | HAMP | 139 | ACTGTCACTCGGTC | InaAs;InaCs;InaTs;dGs;dTs;dCs;dAs; |
| 21 | | | | C | dCs;dTs;dCs;dGs;dGs;InaTs;InaCs;In |
| m08 | | | | | aC-Sup |
| HAMP- | human | HAMP | 140 | ACTCGGTCCCAGAC | InaAs;InaCs;InaTs;dCs;dGs;dGs;dTs; |
| 22 | | | | A | dCs;dCs;dCs;dAs;dGs;InaAs;InaCs;In |
| m08 | | | | | aA-Sup |
| HAMP- | human | HAMP | 141 | AGACACCAGAGCA | InaAs;InaGs;InaAs;dCs;dAs;dCs;dCs; |
| 23 | | | | AG | dAs;dGs;dAs;dGs;dCs;InaAs;InaAs;In |
| m08 | | | | | aG-Sup |
| HAMP- | human | HAMP | 142 | GAGCAAGCTCAAG | InaGs;InaAs;InaGs;dCs;dAs;dAs;dGs; |
| 24 | | | | AC | dCs;dTs;dCs;dAs;dAs;InaGs;InaAs;In |
| m08 | | | | | aC-Sup |
| HAMP- | human | HAMP | 143 | GCAAGACGTAGAA | InaGs;InaCs;InaAs;dAs;dGs;dAs;dCs; |
| 25 | | | | CC | dGs;dTs;dAs;dGs;dAs;InaAs;InaCs;In |
| m08 | | | | | aC-Sup |
| HAMP- | human | HAMP | 144 | AGACGTAGAACCT | InaAs;InaGs;InaAs;dCs;dGs;dTs;dAs; |
| 26 | | | | AC | dGs;dAs;dAs;dCs;dCs;InaTs;InaAs;In |
| m08 | | | | | aC-Sup |
| HAMP- | human | HAMP | 145 | AGAACCTACCTGCC | InaAs;InaGs;InaAs;dAs;dCs;dCs;dTs; |
| 27 | | | | C | dAs;dCs;dCs;dTs;dGs;InaCs;InaCs;In |
| m08 | | | | | aC-Sup |
| HAMP | human- | HAMP | 146 | ACATAGGTCTTGGA | InaAs;InaCs;InaAs;dTs;dAs;dGs;dGs; |
| 28 | | | | A | dTs;dCs;dTs;dTs;dGs;InaGs;InaAs;In |
| m08 | | | | | aA-Sup |
| HAMP- | human | HAMP | 147 | AGGTCTTGGAATA | InaAs;InaGs;InaGs;dTs;dCs;dTs;dTs; |
| 29 | | | | AA | dGs;dGs;dAs;dAs;dTs;InaAs;InaAs;In |
| m08 | | | | | aA-Sup |
| HAMP- | human | HAMP | 148 | TGGAATAAAATGG | InaTs;InaGs;InaGs;dAs;dAs;dTs;dAs; |
| 30 | | | | CT | dAs;dAs;dAs;dTs;dGs;InaGs;InaCs;In |
| m08 | | | | | aT-Sup |
| HAMP- | human | HAMP | 149 | TGGCTGGTTCTTTT | InaTs;InaGs;InaGs;dCs;dTs;dGs;dGs; |
| 31 | | | | G | dTs;dTs;dCs;dTs;dTs;InaTs;InaTs;Ina |
| m08 | | | | | G-Sup |
| HAMP- | human | HAMP | 150 | GTTTTCCAAACCAG | InaGs;InaTs;InaTs;dTs;dTs;dCs;dCs;d |
| 32 | | | | A | As;dAs;dAs;dCs;dCs;InaAs;InaGs;Ina |
| m08 | | | | | A-Sup |
| HAMP- | human | HAMP | 151 | ACCAGAGTGTCTGT | InaAs;InaCs;InaCs;dAs;dGs;dAs;dGs; |
| 33 | | | | T | dTs;dGs;dTs;dCs;dTs;InaGs;InaTs;Ina |
| m08 | | | | | T-Sup |
| HAMP- | human | HAMP | 152 | AGTGTCTGTTGTCC | InaAs;InaGs;InaTs;dGs;dTs;dCs;dTs; |
| 34 | | | | T | dGs;dTs;dTs;dGs;dTs;InaCs;InaCs;In |
| m08 | | | | | aT-Sup |
| HAMP- | human | HAMP | 153 | GTTGTCCTTTCTCTC | InaGs;InaTs;InaTs;dGs;dTs;dCs;dCs; |
| 35 | | | | | dTs;dTs;dTs;dCs;dTs;InaCs;InaTs;Ina |
| m08 | | | | | C-Sup |
| HAMP- | human | HAMP | 154 | CTTTCTCTCTGCCG | InaCs;InaTs;InaTs;dTs;dCs;dTs;dCs;d |
| 36 | | | | A | Ts;dCs;dTs;dGs;dCs;InaCs;InaGs;Ina |
| m08 | | | | | A-Sup |
| HAMP- | human | HAMP | 155 | CTGCCGAGTGTCTG | InaCs;InaTs;InaGs;dCs;dCs;dGs;dAs; |
| 37 | | | | T | dGs;dTs;dGs;dTs;dCs;InaTs;InaGs;In |
| m08 | | | | | aT-Sup |
| NR1H | human | NR1 | 156 | CTCTCCCAAGGTT | InaCs;omeUs;InaCs;omeUs;InaCs;o |
| 4-27 | | H4 | | C | CmeCs;InaCs;omeAs;InaAs;omeGs;In |
| m01 | | | | | aGs;omeUs;InaTs;omeCs;InaC-Sup |
| NR1H | human | NR1 | 157 | AGGTTCCTTTCTAT | InaAs;omeGs;InaGs;omeUs;InaTs;o |
| 4-28 | | H4 | | G | meCs;InaCs;omeUs;InaTs;omeUs;In |
| m01 | | | | | aCs;omeUs;InaAs;omeUs;InaG-Sup |
| NR1H | human | NR1 | 158 | ATGTTTATATCATTT | InaAs;omeUs;InaGs;omeUs;InaTs;o |
| 4-29 | | H4 | | | meUs;InaAs;omeUs;InaAs;omeUs;In |
| m01 | | | | | aCs;omeAs;InaTs;omeUs;InaT-Sup |
| NR1H | human | NR1 | 159 | ATATCATTTAGCAG | InaAs;omeUs;InaAs;omeUs;InaCs;o |
| 4-30 | | H4 | | G | meAs;InaTs;omeUs;InaTs;omeAs;In |
| m01 | | | | | aGs;omeCs;InaAs;omeGs;InaG-Sup |
| NR1H | human | NR1 | 160 | ATTGTTAATGACT | InaAs;omeUs;InaTs;omeGs;InaTs;o |
| 4-31 | | H4 | | A | AmeUs;InaAs;omeAs;InaTs;omeGs;In |
| m01 | | | | | aAs;omeCs;InaTs;omeAs;InaA-Sup |
| NR1H | human | NR1 | 161 | AGCTTCTAGTTCAG | InaAs;omeGs;InaCs;omeUs;InaTs;o |
| 4-32 | | H4 | | T | meCs;InaTs;omeAs;InaGs;omeUs;In |
| m01 | | | | | aTs;omeCs;InaAs;omeGs;InaT-Sup |
| NR1H | human | NR1 | 162 | AGTGATAGAGCTA | InaAs;omeGs;InaTs;omeGs;InaAs;o |
| 4-33 | | H4 | | TT | meUs;InaAs;omeGs;InaAs;omeGs;In |
| m01 | | | | | aCs;omeUs;InaAs;omeUs;InaT-Sup |
| NR1H | human | NR1 | 163 | AGAGAGGGAAGAT | InaAs;omeGs;InaAs;omeGs;InaAs;o |
| 4-34 | | H4 | | GA | meGs;InaGs;omeGs;InaAs;omeAs;In |
| m01 | | | | | aGs;omeAs;InaTs;omeGs;InaA-Sup |
| NR1H | human | NR1 | 164 | AGTTGATGTGTACA | InaAs;omeGs;InaTs;omeUs;InaGs;o |
| 4-35 | | H4 | | G | meAs;InaTs;omeGs;InaTs;omeGs;In |
| m01 | | | | | aTs;omeAs;InaCs;omeAs;InaG-Sup |
| NR1H | human | NR1 | 165 | ACGGGTGCCCAGG | InaAs;omeCs;InaGs;omeGs;InaGs;o |
| 4-36 | | H4 | | AG | meUs;InaGs;omeCs;InaCs;omeCs;In |
| m01 | | | | | aAs;omeGs;InaGs;omeAs;InaG-Sup |
| NR1H | human | NR1 | 166 | CACAAAACGGCCA | InaCs;omeAs;InaCs;omeAs;InaAs;o |
| 4-37 | | H4 | | GA | meAs;InaAs;omeCs;InaGs;omeGs;In |
| m01 | | | | | aCs;omeCs;InaAs;omeGs;InaA-Sup |
| NR1H | human | NR1 | 167 | ATATTGCATATATT | InaAs;omeUs;InaAs;omeUs;InaTs;o |
| 4-38 | | H4 | | T | meGs;InaCs;omeAs;InaTs;omeAs;In |
| m01 | | | | | aTs;omeAs;InaTs;omeUs;InaT-Sup |
| NR1H | human | NR1 | 168 | ATATTTTATTAAAG | InaAs;omeUs;InaAs;omeUs;InaTs;o |
| 4-39 | | H4 | | A | meUs;InaTs;omeAs;InaTs;omeUs;In |
| m01 | | | | | aAs;omeAs;InaAs;omeGs;InaA-Sup |
| NR1H | human | NR1 | 169 | AGAGTTGTATTCAA | InaAs;omeGs;InaAs;omeGs;InaTs;o |
| 4-40 | | H4 | | T | meUs;InaGs;omeUs;InaAs;omeUs;In |
| m01 | | | | | aTs;omeCs;InaAs;omeAs;InaT-Sup |
| NR1H | human | NR1 | 170 | TGTATTCAATCTTG | InaTs;omeGs;InaTs;omeAs;InaTs;om |
| 4-41 | | H4 | | G | eUs;InaCs;omeAs;InaAs;omeUs;InaC |
| m01 | | | | | s;omeUs;InaTs;omeGs;InaG-Sup |
| NR1H | human | NR1 | 171 | CAATCTTGGCAATA | InaCs;omeAs;InaAs;omeUs;InaCs;o |
| 4-42 | | H4 | | A | meUs;InaTs;omeGs;InaGs;omeCs;In |
| m01 | | | | | aAs;omeAs;InaTs;omeAs;InaA-Sup |
| NR1H | human | NR1 | 172 | AGCAAACATAATG | InaAs;omeGs;InaCs;omeAs;InaAs;o |
| 4-43 | | H4 | | GC | meAs;InaCs;omeAs;InaTs;omeAs;In |
| m01 | | | | | aAs;omeUs;InaGs;omeGs;InaC-Sup |
| NR1H | human | NR1 | 173 | ATGGCAACAGGAT | InaAs;omeUs;InaGs;omeGs;InaCs;o |
| 4-44 | | H4 | | TT | meAs;InaAs;omeCs;InaAs;omeGs;In |
| m01 | | | | | aGs;omeAs;InaTs;omeUs;InaT-Sup |
| NR1H | human | NR1 | 174 | TTTTCTTTGGGAAC | InaTs;omeUs;InaTs;omeUs;InaCs;o |
| 4-45 | | H4 | | A | meUs;InaTs;omeUs;InaGs;omeGs;In |
| m01 | | | | | aGs;omeAs;InaAs;omeCs;InaA-Sup |
| NR1H | human | NR1 | 175 | ATTCTAATTGGCAA | InaAs;omeUs;InaTs;omeCs;InaTs;o |
| 4-46 | | H4 | | G | meAs;InaAs;omeUs;InaTs;omeGs;In |
| m01 | | | | | aGs;omeCs;InaAs;omeAs;InaG-Sup |
| NR1H | human | NR1 | 176 | ATTGGCAAGCCCTG | InaAs;omeUs;InaTs;omeGs;InaGs;o |
| 4-47 | | H4 | | T | meCs;InaAs;omeAs;InaGs;omeCs;In |
| m01 | | | | | aCs;omeCs;InaTs;omeGs;InaT-Sup |
| NR1H | human | NR1 | 177 | AGCCCTGTTTGCCT | InaAs;omeGs;InaCs;omeCs;InaCs;o |
| 4-48 | | H4 | | A | meUs;InaGs;omeUs;InaTs;omeUs;In |
| m01 | | | | | aGs;omeCs;InaCs;omeUs;InaA-Sup |
| NR1H | human | NR1 | 178 | CTAATTAAATTGAT | InaCs;omeUs;InaAs;omeAs;InaTs;o |
| 4-49 | | H4 | | T | meUs;InaAs;omeAs;InaAs;omeUs;In |
| m01 | | | | | aTs;omeGs;InaAs;omeUs;InaT-Sup |
| NR1H | human | NR1 | 179 | ATTGTTACTTCAAT | InaAs;omeUs;InaTs;omeGs;InaTs;o |
| 4-50 | | H4 | | T | meUs;InaAs;omeCs;InaTs;omeUs;In |
| m01 | | | | | aCs;omeAs;InaAs;omeUs;InaT-Sup |
| NR1H | human | NR1 | 180 | TTCTATCTGTTGA | InaTs;omeUs;InaCs;omeUs;InaAs;o |
| 4-51 | | H4 | | C | AmeUs;InaCs;omeUs;InaGs;omeUs;In |
| m01 | | | | | aTs;omeGs;InaAs;omeAs;InaC-Sup |
| NR1H | human | NR1 | 181 | CTCTCCCAAGGTTC | InaCs;InaTs;InaCs;dTs;dCs;dCs;dCs;d |
| 4-27 | | H4 | | C | As;dAs;dGs;dGs;dTs;InaTs;InaCs;Ina |
| m08 | | | | | C-Sup |
| NR1H | human | NR1 | 182 | AGGTTCCTTTCTAT | InaAs;InaGs;InaGs;dTs;dTs;dCs;dCs; |
| 4-28 | | H4 | | G | dTs;dTs;dTs;dCs;dTs;InaAs;InaTs;Ina |
| m08 | | | | | G-Sup |
| NR1H | human | NR1 | 183 | ATGTTTATATCATTT | InaAs;InaTs;InaGs;dTs;dTs;dTs;dAs;d |
| 4-29 | | H4 | | | Ts;dAs;dTs;dCs;dAs;InaTs;InaTs;InaT |
| m08 | | | | | -Sup |
| NR1H | human | NR1 | 184 | ATATCATTTAGCAG | InaAs;InaTs;InaAs;dTs;dCs;dAs;dTs;d |
| 4-30 | | H4 | | G | Ts;dTs;dAs;dGs;dCs;InaAs;InaGs;Ina |
| m08 | | | | | G-Sup |
| NR1H | human | NR1 | 185 | ATTGTTAATGACTA | InaAs;InaTs;InaTs;dGs;dTs;dTs;dAs;d |
| 4-31 | | H4 | | A | As;dTs;dGs;dAs;dCs;InaTs;InaAs;Ina |
| m08 | | | | | A-Sup |
| NR1H | human | NR1 | 186 | AGCTTCTAGTTCAG | InaAs;InaGs;InaCs;dTs;dTs;dCs;dTs;d |
| 4-32 | | H4 | | T | As;dGs;dTs;dTs;dCs;InaAs;InaGs;Ina |
| m08 | | | | | T-Sup |
| NR1H | human | NR1 | 187 | AGTGATAGAGCTA | InaAs;InaGs;InaTs;dGs;dAs;dTs;dAs; |
| 4-33 | | H4 | | TT | dGs;dAs;dGs;dCs;dTs;InaAs;InaTs;In |
| m08 | | | | | aT-Sup |
| NR1H | human | NR1 | 188 | AGAGAGGGAAGAT | InaAs;InaGs;InaAs;dGs;dAs;dGs;dGs; |
| 4-34 | | H4 | | GA | dGs;dAs;dAs;dGs;dAs;InaTs;InaGs;In |
| m08 | | | | | aA-Sup |
| NR1H | human | NR1 | 189 | AGTTGATGTGTACA | InaAs;InaGs;InaTs;dTs;dGs;dAs;dTs; |
| 4-35 | | H4 | | G | dGs;dTs;dGs;dTs;dAs;InaCs;InaAs;In |
| m08 | | | | | aG-Sup |
| NR1H | human | NR1 | 190 | ACGGGTGCCCAGG | InaAs;InaCs;InaGs;dGs;dGs;dTs;dGs; |
| 4-36 | | H4 | | AG | dCs;dCs;dCs;dAs;dGs;InaGs;InaAs;In |
| m08 | | | | | aG-Sup |
| NR1H | human | NR1 | 191 | CACAAAACGGCCA | InaCs;InaAs;InaCs;dAs;dAs;dAs;dAs; |
| 4-37 | | H4 | | GA | dCs;dGs;dGs;dCs;dCs;InaAs;InaGs;In |
| m08 | | | | | aA-Sup |
| NR1H | human | NR1 | 192 | ATATTGCATATATT | InaAs;InaTs;InaAs;dTs;dTs;dGs;dCs; |
| 4-38 | | H4 | | T | dAs;dTs;dAs;dTs;dAs;InaTs;InaTs;Ina |
| m08 | | | | | T-Sup |
| NR1H | human | NR1 | 193 | ATATTTTATTAAAG | InaAs;InaTs;InaAs;dTs;dTs;dTs;dTs;d |
| 4-39 | | H4 | | A | As;dTs;dTs;dAs;dAs;InaAs;InaGs;Ina |
| m08 | | | | | A-Sup |
| NR1H | human | NR1 | 194 | AGAGTTGTATTCAA | InaAs;InaGs;InaAs;dGs;dTs;dTs;dGs; |
| 4-40 | | H4 | | T | dTs;dAs;dTs;dTs;dCs;InaAs;InaAs;Ina |
| m08 | | | | | T-Sup |
| NR1H | human | NR1 | 195 | TGTATTCAATCTTG | InaTs;InaGs;InaTs;dAs;dTs;dTs;dCs;d |
| 4-41 | | H4 | | G | As;dAs;dTs;dCs;dTs;InaTs;InaGs;Ina |
| m08 | | | | | G-Sup |
| NR1H | human | NR1 | 196 | CAATCTTGGCAATA | InaCs;InaAs;InaAs;dTs;dCs;dTs;dTs;d |
| 4-42 | | H4 | | A | Gs;dGs;dCs;dAs;dAs;InaTs;InaAs;Ina |
| m08 | | | | | A-Sup |
| NR1H | human | NR1 | 197 | AGCAAACATAATG | InaAs;InaGs;InaCs;dAs;dAs;dAs;dCs; |
| 4-43 | | H4 | | GC | dAs;dTs;dAs;dAs;dTs;InaGs;InaGs;In |
| m08 | | | | | aC-Sup |
| NR1H | human | NR1 | 198 | ATGGCAACAGGA | InaAs;InaTs;InaGs;dGs;dCs;dAs;dAs; |
| 4-44 | | H4 | | TT | TdCs;dAs;dGs;dGs;dAs;InaTs;InaTs;In |
| m08 | | | | | aT-Sup |
| NR1H | human | NR1 | 199 | TTTTCTTTGGGAAC | InaTs;InaTs;InaTs;dTs;dCs;dTs;dTs;d |
| 4-45 | | H4 | | A | Ts;dGs;dGs;dGs;dAs;InaAs;InaCs;Ina |
| m08 | | | | | A-Sup |
| NR1H | human | NR1 | 200 | ATTCTAATTGGCAA | InaAs;InaTs;InaTs;dCs;dTs;dAs;dAs;d |
| 4-46 | | H4 | | G | Ts;dTs;dGs;dGs;dCs;InaAs;InaAs;Ina |
| m08 | | | | | G-Sup |
| NR1H | human | NR1 | 201 | ATTGGCAAGCCCTG | InaAs;InaTs;InaTs;dGs;dGs;dCs;dAs; |
| 4-47 | | H4 | | T | dAs;dGs;dCs;dCs;dCs;InaTs;InaGs;In |
| m08 | | | | | aT-Sup |
| NR1H | human | NR1 | 202 | AGCCCTGTTTGCCT | InaAs;InaGs;InaCs;dCs;dCs;dTs;dGs; |
| 4-48 | | H4 | | A | dTs;dTs;dTs;dGs;dCs;InaCs;InaTs;Ina |
| m08 | | | | | A-Sup |
| NR1H | human | NR1 | 203 | CTAATTAAATTGATA | InaCs;InaTs;InaAs;dAs;dTs;dTs;dAs;d |
| 4-49 | | H4 | | T | s;dAs;dTs;dTs;dGs;InaAs;InaTs;Ina |
| m08 | | | | | T-Sup |
| NR1H | human | NR1 | 204 | ATTGTTACTTCAATC | InaAs;InaTs;InaTs;dGs;dTs;dTs;dAs;d |
| 4-50 | | H4 | | T | s;dTs;dTs;dCs;dAs;InaAs;InaTs;InaT |
| m08 | | | | | -Sup |
| NR1H | human | NR1 | 205 | TTCTATCTGTTGAAT | InaTs;InaTs;InaCs;dTs;dAs;dTs;dCs;d |
| 4-51 | | H4 | | C | s;dGs;dTs;dTs;dGs;InaAs;InaAs;Ina |
| m08 | | | | | C-Sup |
| THRB- | human | THRB | 206 | GAATATAGTGGGC | InaGs;omeAs;InaAs;omeUs;InaAs;o |
| 91 | | | | GT | meUs;InaAs;omeGs;InaTs;omeGs;In |
| m01 | | | | | aGs;omeGs;InaCs;omeGs;InaT-Sup |
| THRB- | human | THRB | 207 | AGTGGGCGTAGAT | InaAs;omeGs;InaTs;omeGs;InaGs;o |
| 92 | | | | AA | meGs;InaCs;omeGs;InaTs;omeAs;In |
| m01 | | | | | aGs;omeAs;InaTs;omeAs;InaA-Sup |
| THRB- | human | THRB | 208 | CGTAGATAAACTCA | InaCs;omeGs;InaTs;omeAs;InaGs;o |
| 93 | | | | T | meAs;InaTs;omeAs;InaAs;omeAs;In |
| m01 | | | | | aCs;omeUs;InaCs;omeAs;InaT-Sup |
| THRB- | human | THRB | 209 | TAAACTCATAAGCT | InaTs;omeAs;InaAs;omeAs;InaCs;o |
| 94 | | | | T | meUs;InaCs;omeAs;InaTs;omeAs;In |
| m01 | | | | | aAs;omeGs;InaCs;omeUs;InaT-Sup |
| THRB- | human | THRB | 210 | CATAAGCTTAAATT | InaCs;omeAs;InaTs;omeAs;InaAs;o |
| 95 | | | | C | meGs;InaCs;omeUs;InaTs;omeAs;In |
| m01 | | | | | aAs;omeAs;InaTs;omeUs;InaC-Sup |
| THRB- | human | THRB | 211 | AAGCTTATAACAGA | InaAs;omeAs;InaGs;omeCs;InaTs;o |
| 96 | | | | T | meUs;InaAs;omeUs;InaAs;omeAs;In |
| m01 | | | | | aCs;omeAs;InaGs;omeAs;InaT-Sup |
| THRB- | human | THRB | 212 | ATAACAGATATATT | InaAs;omeUs;InaAs;omeAs;InaCs;o |
| 97 | | | | T | meAs;InaGs;omeAs;InaTs;omeAs;In |
| m01 | | | | | aTs;omeAs;InaTs;omeUs;InaT-Sup |
| THRB- | human | THRB | 213 | GATATATTTTCCTG | InaGs;omeAs;InaTs;omeAs;InaTs;o |
| 98 | | | | T | meAs;InaTs;omeUs;InaTs;omeUs;In |
| m01 | | | | | aCs;omeCs;InaTs;omeGs;InaT-Sup |
| THRB- | human | THRB | 214 | TTTCCTGTCTCTTTC | InaTs;omeUs;InaTs;omeCs;InaCs;om |
| 99 | | | | | eUs;InaGs;omeUs;InaCs;omeUs;Ina |
| m01 | | | | | Cs;omeUs;InaTs;omeUs;InaC-Sup |
| THRB- | human | THRB | 215 | ATGGATTTTTACAT | InaAs;omeUs;InaGs;omeGs;InaAs;o |
| 100 | | | | A | meUs;InaTs;omeUs;InaTs;omeUs;In |
| m01 | | | | | aAs;omeCs;InaAs;omeUs;InaA-Sup |
| THRB- | human | THRB | 216 | TGTATGCAGATATA | InaTs;omeGs;InaTs;omeAs;InaTs;om |
| 101 | | | | A | eGs;InaCs;omeAs;InaGs;omeAs;InaT |
| m01 | | | | | s;omeAs;InaTs;omeAs;InaA-Sup |
| THRB- | human | THRB | 217 | CTGTAATTATGAAT | InaCs;omeUs;InaGs;omeUs;InaAs;o |
| 102 | | | | A | meAs;InaTs;omeUs;InaAs;omeUs;In |
| m01 | | | | | aGs;omeAs;InaAs;omeUs;InaA-Sup |
| THRB- | human | THRB | 218 | TACATAGGCAAAG | InaTs;omeAs;InaCs;omeAs;InaTs;om |
| 103 | | | | AG | eAs;InaGs;omeGs;InaCs;omeAs;InaA |
| m01 | | | | | s;omeAs;InaGs;omeAs;InaG-Sup |
| THRB- | human | THRB | 219 | CAAAGAGTTGCCT | InaCs;omeAs;InaAs;omeAs;InaGs;o |
| 104 | | | | GC | meAs;InaGs;omeUs;InaTs;omeGs;In |
| m01 | | | | | aCs;omeCs;InaTs;omeGs;InaC-Sup |
| THRB- | human | THRB | 220 | CCAGCCGCTTCCTG | InaCs;omeCs;InaAs;omeGs;InaCs;o |
| 105 | | | | C | meCs;InaGs;omeCs;InaTs;omeUs;In |
| m01 | | | | | aCs;omeCs;InaTs;omeGs;InaC-Sup |
| THRB- | human | THRB | 221 | TAGACATGGATGA | InaTs;omeAs;InaGs;omeAs;InaCs;o |
| 106 | | | | AA | meAs;InaTs;omeGs;InaGs;omeAs;In |
| m01 | | | | | aTs;omeGs;InaAs;omeAs;InaA-Sup |
| THRB- | human | THRB | 222 | GATGAAATTGCCCC | InaGs;omeAs;InaTs;omeGs;InaAs;o |
| 107 | | | | T | meAs;InaAs;omeUs;InaTs;omeGs;In |
| m01 | | | | | aCs;omeCs;InaCs;omeCs;InaT-Sup |
| THRB- | human | THRB | 223 | TGCCCCTTGAATGC | InaTs;omeGs;InaCs;omeCs;InaCs;o |
| 108 | | | | G | meCs;InaTs;omeUs;InaGs;omeAs;In |
| m01 | | | | | aAs;omeUs;InaGs;omeCs;InaG-Sup |
| THRB- | human | THRB | 224 | TGAATGCGGGTAC | InaTs;omeGs;InaAs;omeAs;InaTs;o |
| 109 | | | | TT | meGs;InaCs;omeGs;InaGs;omeGs;In |
| m01 | | | | | aTs;omeAs;InaCs;omeUs;InaT-Sup |
| THRB- | human | THRB | 225 | GTACTTGAAACTAT | InaGs;omeUs;InaAs;omeCs;InaTs;o |
| 110 | | | | T | meUs;InaGs;omeAs;InaAs;omeAs;In |
| m01 | | | | | aCs;omeUs;InaAs;omeUs;InaT-Sup |
| THRB- | human | THRB | 226 | ACTATTGCATTTCG | InaAs;omeCs;InaTs;omeAs;InaTs;om |
| 111 | | | | T | eUs;InaGs;omeCs;InaAs;omeUs;InaT |
| m01 | | | | | s;omeUs;InaCs;omeGs;InaT-Sup |
| THRB- | human | THRB | 227 | CGTTCTCCGGTCCT | InaCs;omeGs;InaTs;omeUs;InaCs;o |
| 112 | | | | G | meUs;InaCs;omeCs;InaGs;omeGs;In |
| m01 | | | | | aTs;omeCs;InaCs;omeUs;InaG-Sup |
| THRB- | human | THRB | 228 | CTGTGATGTGAATG | InaCs;omeUs;InaGs;omeUs;InaGs;o |
| 113 | | | | C | meAs;InaTs;omeGs;InaTs;omeGs;In |
| m01 | | | | | aAs;omeAs;InaTs;omeGs;InaC-Sup |
| THRB- | human | THRB | 229 | GTTCGAGGATTAG | InaGs;omeUs;InaTs;omeCs;InaGs;o |
| 114 | | | | AC | meAs;InaGs;omeGs;InaAs;omeUs;In |
| m01 | | | | | aTs;omeAs;InaGs;omeAs;InaC-Sup |
| THRB- | human | THRB | 230 | ATTAGACTGACTGG | InaAs;omeUs;InaTs;omeAs;InaGs;o |
| 115 | | | | A | meAs;InaCs;omeUs;InaGs;omeAs;In |
| m01 | | | | | aCs;omeUs;InaGs;omeGs;InaA-Sup |
| THRB- | human | THRB | 231 | ACTGGATTCATTCT | InaAs;omeCs;InaTs;omeGs;InaGs;o |
| 116 | | | | C | meAs;InaTs;omeUs;InaCs;omeAs;In |
| m01 | | | | | aTs;omeUs;InaCs;omeUs;InaC-Sup |
| THRB- | human | THRB | 232 | ATTCTCATAATTCCT | InaAs;omeUs;InaTs;omeCs;InaTs;o |
| 117 | | | | | meCs;InaAs;omeUs;InaAs;omeAs;In |
| m01 | | | | | aTs;omeUs;InaCs;omeCs;InaT-Sup |
| THRB- | human | THRB | 233 | ATTCCTACAGCACT | InaAs;omeUs;InaTs;omeCs;InaCs;o |
| 118 | | | | A | meUs;InaAs;omeCs;InaAs;omeGs;In |
| m01 | | | | | aCs;omeAs;InaCs;omeUs;InaA-Sup |
| THRB- | human | THRB | 234 | TCATTTCATTCCATT | InaTs;omeCs;InaAs;omeUs;InaTs;o |
| 119 | | | | | meUs;InaCs;omeAs;InaTs;omeUs;In |
| m01 | | | | | aCs;omeCs;InaAs;omeUs;InaT-Sup |
| THRB- | human | THRB | 235 | TCCATTGCCTAGCT | InaTs;omeCs;InaCs;omeAs;InaTs;om |
| 120 | | | | C | eUs;InaGs;omeCs;InaCs;omeUs;Ina |
| m01 | | | | | As;omeGs;InaCs;omeUs;InaC-Sup |
| THRB- | human | THRB | 236 | ACCAGGTCACCGG | InaAs;omeCs;InaCs;omeAs;InaGs;o |
| 121 | | | | TT | meGs;InaTs;omeCs;InaAs;omeCs;In |
| m01 | | | | | aCs;omeGs;InaGs;omeUs;InaT-Sup |
| THRB- | human | THRB | 237 | CGCAGTAGCTTCCT | InaCs;omeGs;InaCs;omeAs;InaGs;o |
| 122 | | | | A | meUs;InaAs;omeGs;InaCs;omeUs;In |
| m01 | | | | | aTs;omeCs;InaCs;omeUs;InaA-Sup |
| THRB- | human | THRB | 238 | CAAGGAGTTGACA | InaCs;omeAs;InaAs;omeGs;InaGs;o |
| 123 | | | | TT | meAs;InaGs;omeUs;InaTs;omeGs;In |
| m01 | | | | | aAs;omeCs;InaAs;omeUs;InaT-Sup |
| THRB- | | THRB | 239 | ACATTTTGCAGGAC | InaAs;omeCs;InaAs;omeUs;InaTs;o |
| 124 | human | | | T | meUs;InaTs;omeGs;InaCs;omeAs;In |
| m01 | | | | | aGs;omeGs;InaAs;omeCs;InaT-Sup |
| THRB- | human | THRB | 240 | CAAGGAAGGCGCA | InaCs;omeAs;InaAs;omeGs;InaGs;o |
| 125 | | | | CA | meAs;InaAs;omeGs;InaGs;omeCs;In |
| m01 | | | | | aGs;omeCs;InaAs;omeCs;InaA-Sup |
| THRB- | human | THRB | 241 | ATTAACTTTGCATG | InaAs;omeUs;InaTs;omeAs;InaAs;o |
| 126 | | | | A | meCs;InaTs;omeUs;InaTs;omeGs;In |
| m01 | | | | | aCs;omeAs;InaTs;omeGs;InaA-Sup |
| THRB- | human | THRB | 242 | TGAATAATGTGAGT | InaTs;omeGs;InaAs;omeAs;InaTs;o |
| 127 | | | | G | meAs;InaAs;omeUs;InaGs;omeUs;In |
| m01 | | | | | aGs;omeAs;InaGs;omeUs;InaG-Sup |
| THRB- | human | THRB | 243 | GTAATTTGGCTAGA | InaGs;omeUs;InaAs;omeAs;InaTs;o |
| 128 | | | | G | meUs;InaTs;omeGs;InaGs;omeCs;In |
| m01 | | | | | aTs;omeAs;InaGs;omeAs;InaG-Sup |
| THRB- | human | THRB | 244 | ACAGTTCCAACTGT | InaAs;omeCs;InaAs;omeGs;InaTs;o |
| 129 | | | | C | meUs;InaCs;omeCs;InaAs;omeAs;In |
| m01 | | | | | aCs;omeUs;InaGs;omeUs;InaC-Sup |
| THRB- | human | THRB | 245 | ATCACTCTGAACAT | InaAs;InaTs;InaCs;dAs;dCs;dTs;dCs;d |
| 130 | | | | T | Ts;dGs;dAs;dAs;dCs;InaAs;InaTs;Ina |
| m08 | | | | | T-Sup |
| THRB- | human | THRB | 246 | GAGCCTATATTCAT | InaGs;InaAs;InaGs;dCs;dCs;dTs;dAs; |
| 131 | | | | A | dTs;dAs;dTs;dTs;dCs;InaAs;InaTs;Ina |
| m08 | | | | | A-Sup |
| THRB- | human | THRB | 247 | ATGCATTTAGGTCT | InaAs;InaTs;InaGs;dCs;dAs;dTs;dTs; |
| 132 | | | | A | dTs;dAs;dGs;dGs;dTs;InaCs;InaTs;In |
| m08 | | | | | aA-Sup |
| THRB- | human | THRB | 248 | ATGCTGTGATAGA | InaAs;InaTs;InaGs;dCs;dTs;dGs;dTs; |
| 133 | | | | GT | dGs;dAs;dTs;dAs;dGs;InaAs;InaGs;In |
| m08 | | | | | aT-Sup |
| THRB- | human | THRB | 249 | CATATTAATGCATT | InaCs;InaAs;InaTs;dAs;dTs;dTs;dAs;d |
| 134 | | | | T | As;dTs;dGs;dCs;dAs;InaTs;InaTs;InaT |
| m08 | | | | | -Sup |
| THRB- | human | THRB | 250 | TCATCAGCCTGATT | InaTs;InaCs;InaAs;dTs;dCs;dAs;dGs; |
| 135 | | | | A | dCs;dCs;dTs;dGs;dAs;InaTs;InaTs;Ina |
| m08 | | | | | A-Sup |
| THRB- | human | THRB | 251 | TACGGAGTGGACA | InaTs;InaAs;InaCs;dGs;dGs;dAs;dGs; |
| 136 | | | | GT | dTs;dGs;dGs;dAs;dCs;InaAs;InaGs;In |
| m08 | | | | | aT-Sup |
| THRB- | human | THRB | 252 | CAATCGCAGCGGC | InaCs;InaAs;InaAs;dTs;dCs;dGs;dCs; |
| 137 | | | | TC | dAs;dGs;dCs;dGs;dGs;InaCs;InaTs;In |
| m08 | | | | | aC-Sup |
| THRB- | human | THRB | 253 | CAGCTGTTGACATG | InaCs;InaAs;InaGs;dCs;dTs;dGs;dTs; |
| 138 | | | | T | dTs;dGs;dAs;dCs;dAs;InaTs;InaGs;In |
| m08 | | | | | aT-Sup |
| THRB- | human | THRB | 254 | ATGGAGTTTGGCAT | InaAs;InaTs;InaGs;dGs;dAs;dGs;dTs; |
| 139 | | | | C | dTs;dTs;dGs;dGs;dCs;InaAs;InaTs;In |
| m08 | | | | | aC-Sup |
| THRB- | human | THRB | 255 | CATGATGAGGAAG | InaCs;InaAs;InaTs;dGs;dAs;dTs;dGs; |
| 140 | | | | TT | dAs;dGs;dGs;dAs;dAs;InaGs;InaTs;In |
| m08 | | | | | aT-Sup |
| THRB- | human | THRB | 256 | CTCTGTTCCTCAAA | InaCs;InaTs;InaCs;dTs;dGs;dTs;dTs;d |
| 141 | | | | C | Cs;dCs;dTs;dCs;dAs;InaAs;InaAs;Ina |
| m08 | | | | | C-Sup |
| THRB- | human | THRB | 257 | GAATATAGTGGGC | InaGs;InaAs;InaAs;dTs;dAs;dTs;dAs; |
| 91 | | | | GT | dGs;dTs;dGs;dGs;dGs;InaCs;InaGs;In |
| m08 | | | | | aT-Sup |
| THRB- | human | THRB | 258 | AGTGGGCGTAGAT | InaAs;InaGs;InaTs;dGs;dGs;dGs;dCs; |
| 92 | | | | AA | dGs;dTs;dAs;dGs;dAs;InaTs;InaAs;In |
| m08 | | | | | aA-Sup |
| THRB- | human | THRB | 259 | CGTAGATAAACTCA | InaCs;InaGs;InaTs;dAs;dGs;dAs;dTs; |
| 93 | | | | T | dAs;dAs;dAs;dCs;dTs;InaCs;InaAs;In |
| m08 | | | | | aT-Sup |
| THRB- | human | THRB | 260 | TAAACTCATAAGCT | InaTs;InaAs;InaAs;dAs;dCs;dTs;dCs; |
| 94 | | | | T | dAs;dTs;dAs;dAs;dGs;InaCs;InaTs;In |
| m08 | | | | | aT-Sup |
| THRB- | human | THRB | 261 | CATAAGCTTAAATT | InaCs;InaAs;InaTs;dAs;dAs;dGs;dCs; |
| 95 | | | | C | dTs;dTs;dAs;dAs;dAs;InaTs;InaTs;Ina |
| m08 | | | | | C-Sup |
| THRB- | human | THRB | 262 | AAGCTTATAACAGA | InaAs;InaAs;InaGs;dCs;dTs;dTs;dAs; |
| 96 | | | | T | dTs;dAs;dAs;dCs;dAs;InaGs;InaAs;In |
| m08 | | | | | aT-Sup |
| THRB- | human | THRB | 263 | ATAACAGATATATT | InaAs;InaTs;InaAs;dAs;dCs;dAs;dGs; |
| 97 | | | | T | dAs;dTs;dAs;dTs;dAs;InaTs;InaTs;Ina |
| m08 | | | | | T-Sup |
| THRB- | human | THRB | 264 | GATATATTTTCCTG | InaGs;InaAs;InaTs;dAs;dTs;dAs;dTs; |
| 98 | | | | T | dTs;dTs;dTs;dCs;dCs;InaTs;InaGs;Ina |
| m08 | | | | | T-Sup |
| THRB- | human | THRB | 265 | TTTCCTGTCTCTTTC | InaTs;InaTs;InaTs;dCs;dCs;dTs;dGs;d |
| 99 | | | | | Ts;dCs;dTs;dCs;dTs;InaTs;InaTs;InaC |
| m08 | | | | | -Sup |
| THRB- | human | THRB | 266 | ATGGATTTTTACAT | InaAs;InaTs;InaGs;dGs;dAs;dTs;dTs; |
| 100 | | | | A | dTs;dTs;dTs;dAs;dCs;InaAs;InaTs;Ina |
| m08 | | | | | A-Sup |
| THRB- | human | THRB | 267 | TGTATGCAGATATA | InaTs;InaGs;InaTs;dAs;dTs;dGs;dCs; |
| 101 | | | | A | dAs;dGs;dAs;dTs;dAs;InaTs;InaAs;In |
| m08 | | | | | aA-Sup |
| THRB- | human | THRB | 268 | CTGTAATTATGAAT | InaCs;InaTs;InaGs;dTs;dAs;dAs;dTs; |
| 102 | | | | A | dTs;dAs;dTs;dGs;dAs;InaAs;InaTs;In |
| m08 | | | | | aA-Sup |
| THRB- | human | THRB | 269 | TACATAGGCAAAG | InaTs;InaAs;InaCs;dAs;dTs;dAs;dGs; |
| 103 | | | | AG | dGs;dCs;dAs;dAs;dAs;InaGs;InaAs;In |
| m08 | | | | | aG-Sup |
| THRB- | human | THRB | 270 | CAAAGAGTTGCCT | InaCs;InaAs;InaAs;dAs;dGs;dAs;dGs; |
| 104 | | | | GC | dTs;dTs;dGs;dCs;dCs;InaTs;InaGs;In |
| m08 | | | | | aC-Sup |
| THRB- | human | THRB | 271 | CCAGCCGCTTCCTG | InaCs;InaCs;InaAs;dGs;dCs;dCs;dGs; |
| 105 | | | | C | dCs;dTs;dTs;dCs;dCs;InaTs;InaGs;Ina |
| m08 | | | | | C-Sup |
| THRB- | human | THRB | 272 | TAGACATGGATGA | InaTs;InaAs;InaGs;dAs;dCs;dAs;dTs; |
| 106 | | | | AA | dGs;dGs;dAs;dTs;dGs;InaAs;InaAs;In |
| m08 | | | | | aA-Sup |
| THRB- | human | THRB | 273 | GATGAAATTGCCCC | InaGs;InaAs;InaTs;dGs;dAs;dAs;dAs; |
| 107 | | | | T | dTs;dTs;dGs;dCs;dCs;InaCs;InaCs;Ina |
| m08 | | | | | T-Sup |
| THRB- | human | THRB | 274 | TGCCCCTTGAATGC | InaTs;InaGs;InaCs;dCs;dCs;dCs;dTs;d |
| 108 | | | | G | Ts;dGs;dAs;dAs;dTs;InaGs;InaCs;Ina |
| m08 | | | | | G-Sup |
| THRB- | human | THRB | 275 | TGAATGCGGGTAC | InaTs;InaGs;InaAs;dAs;dTs;dGs;dCs; |
| 109 | | | | TT | dGs;dGs;dGs;dTs;dAs;InaCs;InaTs;In |
| m08 | | | | | aT-Sup |
| THRB- | human | THRB | 276 | GTACTTGAAACTAT | InaGs;InaTs;InaAs;dCs;dTs;dTs;dGs; |
| 110 | | | | T | dAs;dAs;dAs;dCs;dTs;InaAs;InaTs;In |
| m08 | | | | | aT-Sup |
| THRB- | human | THRB | 277 | ACTATTGCATTTCG | InaAs;InaCs;InaTs;dAs;dTs;dTs;dGs; |
| 111 | | | | T | dCs;dAs;dTs;dTs;dTs;InaCs;InaGs;Ina |
| m08 | | | | | T-Sup |
| THRB- | human | THRB | 278 | CGTTCTCCGGTCCT | InaCs;InaGs;InaTs;dTs;dCs;dTs;dCs;d |
| 112 | | | | G | Cs;dGs;dGs;dTs;dCs;InaCs;InaTs;Ina |
| m08 | | | | | G-Sup |
| THRB- | human | THRB | 279 | CTGTGATGTGAATG | InaCs;InaTs;InaGs;dTs;dGs;dAs;dTs; |
| 113 | | | | C | dGs;dTs;dGs;dAs;dAs;InaTs;InaGs;In |
| m08 | | | | | aC-Sup |
| THRB- | human | THRB | 280 | GTTCGAGGATTAG | InaGs;InaTs;InaTs;dCs;dGs;dAs;dGs; |
| 114 | | | | AC | dGs;dAs;dTs;dTs;dAs;InaGs;InaAs;In |
| m08 | | | | | aC-Sup |
| THRB- | human | THRB | | ATTAGACTGACTGG | InaAs;InaTs;InaTs;dAs;dGs;dAs;dCs; |
| 115 | | | 281 | A | dTs;dGs;dAs;dCs;dTs;InaGs;InaGs;In |
| m08 | | | | | aA-Sup |
| THRB- | human | THRB | | ACTGGATTCATTCT | InaAs;InaCs;InaTs;dGs;dGs;dAs;dTs; |
| 116 | | | 282 | C | dTs;dCs;dAs;dTs;dTs;InaCs;InaTs;Ina |
| m08 | | | | | C-Sup |
| THRB- | human | THRB | 283 | ATTCTCATAATTCCT | InaAs;InaTs;InaTs;dCs;dTs;dCs;dAs;d |
| 117 | | | | | Ts;dAs;dAs;dTs;dTs;InaCs;InaCs;InaT |
| m08 | | | | | -Sup |
| THRB- | human | THRB | 284 | ATTCCTACAGCACT | InaAs;InaTs;InaTs;dCs;dCs;dTs;dAs;d |
| 118 | | | | A | Cs;dAs;dGs;dCs;dAs;InaCs;InaTs;Ina |
| m08 | | | | | A-Sup |
| THRB- | human | THRB | 285 | TCATTTCATTCCATT | InaTs;InaCs;InaAs;dTs;dTs;dTs;dCs;d |
| 119 | | | | | As;dTs;dTs;dCs;dCs;InaAs;InaTs;InaT |
| m08 | | | | | -Sup |
| THRB- | human | THRB | 286 | TCCATTGCCTAGCT | InaTs;InaCs;InaCs;dAs;dTs;dTs;dGs;d |
| 120 | | | | C | Cs;dCs;dTs;dAs;dGs;InaCs;InaTs;Ina |
| m08 | | | | | C-Sup |
| THRB- | human | THRB | 287 | ACCAGGTCACCGG | InaAs;InaCs;InaCs;dAs;dGs;dGs;dTs; |
| 121 | | | | TT | dCs;dAs;dCs;dCs;dGs;InaGs;InaTs;In |
| m08 | | | | | aT-Sup |
| THRB- | human | THRB | 288 | CGCAGTAGCTTCCT | InaCs;InaGs;InaCs;dAs;dGs;dTs;dAs; |
| 122 | | | | A | dGs;dCs;dTs;dTs;dCs;InaCs;InaTs;Ina |
| m08 | | | | | A-Sup |
| THRB- | human | THRB | 289 | CAAGGAGTTGACA | InaCs;InaAs;InaAs;dGs;dGs;dAs;dGs; |
| 123 | | | | TT | dTs;dTs;dGs;dAs;dCs;InaAs;InaTs;In |
| m08 | | | | | aT-Sup |
| THRB- | human | THRB | 290 | ACATTTTGCAGGAC | InaAs;InaCs;InaAs;dTs;dTs;dTs;dTs;d |
| 124 | | | | T | Gs;dCs;dAs;dGs;dGs;InaAs;InaCs;Ina |
| m08 | | | | | T-Sup |
| THRB- | human | THRB | 291 | CAAGGAAGGCGCA | InaCs;InaAs;InaAs;dGs;dGs;dAs;dAs; |
| 125 | | | | CA | dGs;dGs;dCs;dGs;dCs;InaAs;InaCs;In |
| m08 | | | | | aA-Sup |
| THRB- | human | THRB | 292 | ATTAACTTTGCATG | InaAs;InaTs;InaTs;dAs;dAs;dCs;dTs;d |
| 126 | | | | A | Ts;dTs;dGs;dCs;dAs;InaTs;InaGs;Ina |
| m08 | | | | | A-Sup |
| THRB- | human | THRB | 293 | TGAATAATGTGAGT | InaTs;InaGs;InaAs;dAs;dTs;dAs;dAs; |
| 127 | | | | G | dTs;dGs;dTs;dGs;dAs;InaGs;InaTs;In |
| m08 | | | | | aG-Sup |
| THRB- | human | THRB | 294 | GTAATTTGGCTAGA | InaGs;InaTs;InaAs;dAs;dTs;dTs;dTs;d |
| 128 | | | | G | Gs;dGs;dCs;dTs;dAs;InaGs;InaAs;Ina |
| m08 | | | | | G-Sup |
| THRB- | human | THRB | 295 | ACAGTTCCAACTGT | InaAs;InaCs;InaAs;dGs;dTs;dTs;dCs; |
| 129 | | | | C | dCs;dAs;dAs;dCs;dTs;InaGs;InaTs;In |
| m08 | | | | | aC-Sup |

TABLE 4
A listing of oligonucleotide modifications.

| Symbol | Feature Description |
|---|---|
| bio | 5′ biotin |
| dAs | DNA w/3′ thiophosphate |
| dCs | DNA w/3′ thiophosphate |
| dGs | DNA w/3′ thiophosphate |
| dTs | DNA w/3′ thiophosphate |
| dG | DNA |
| enaAs | ENA w/3′ thiophosphate |
| enaCs | ENA w/3′ thiophosphate |
| enaGs | ENA w/3′ thiophosphate |
| enaTs | ENA w/3′ thiophosphate |
| fluAs | 2′-fluoro w/3′ thiophosphate |
| fluCs | 2′-fluoro w/3′ thiophosphate |
| fluGs | 2′-fluoro w/3′ thiophosphate |
| fluUs | 2′-fluoro w/3′ thiophosphate |
| lnaAs | LNA w/3′ thiophosphate |
| lnaCs | LNA w/3′ thiophosphate |
| lnaGs | LNA w/3′ thiophosphate |
| lnaTs | LNA w/3′ thiophosphate |
| omeAs | 2′-OMe w/3′ thiophosphate |
| omeCs | 2′-OMe w/3′ thiophosphate |
| omeGs | 2′-OMe w/3′ thiophosphate |
| omeTs | 2′-OMe w/3′ thiophosphate |
| lnaAs-Sup | LNA w/3′ thiophosphate at 3′ terminus |
| lnaCs-Sup | LNA w/3′ thiophosphate at 3′ terminus |
| lnaGs-Sup | LNA w/3′ thiophosphate at 3′ terminus |
| lnaTs-Sup | LNA w/3′ thiophosphate at 3′ terminus |
| lnaA-Sup | LNA w/3′ OH at 3′ terminus |
| lnaC-Sup | LNA w/3′ OH at 3′ terminus |
| lnaG-Sup | LNA w/3′ OH at 3′ terminus |
| lnaT-Sup | LNA w/3′ OH at 3′ terminus |
| omeA-Sup | 2′-OMe w/3′ OH at 3′ terminus |
| omeC-Sup | 2′-OMe w/3′ OH at 3′ terminus |
| omeG-Sup | 2′-OMe w/3′ OH at 3′ terminus |
| omeU-Sup | 2′-OMe w/3′ OH at 3′ terminus |
| dAs-Sup | DNA w/3′ thiophosphate at 3′ terminus |
| dCs-Sup | DNA w/3′ thiophosphate at 3′ terminus |
| dGs-Sup | DNA w/3′ thiophosphate at 3′ terminus |
| dTs-Sup | DNA w/3′ thiophosphate at 3′ terminus |
| dA-Sup | DNA w/3′ OH at 3′ terminus |
| dC-Sup | DNA w/3′ OH at 3′ terminus |
| dG-Sup | DNA w/3′ OH at 3′ terminus |
| dT-Sup | DNA w/3′ OH at 3′ terminus |
| Symbol | Feature Description |
| bio | 5′ biotin |
| dAs | DNA w/3′ thiophosphate |
| dCs | DNA w/3′ thiophosphate |
| dGs | DNA w/3′ thiophosphate |
| dTs | DNA w/3′ thiophosphate |
| dG | DNA |
| enaAs | ENA w/3′ thiophosphate |

The suffix “Sup” in Table 4 indicates that a 3′ end nucleotide may, for synthesis purposes, be conjugated to a solid support. It should be appreciated that in general when conjugated to a solid support for synthesis, the synthesized oligonucleotide is released such that the solid support is not part of the final oligonucleotide product.

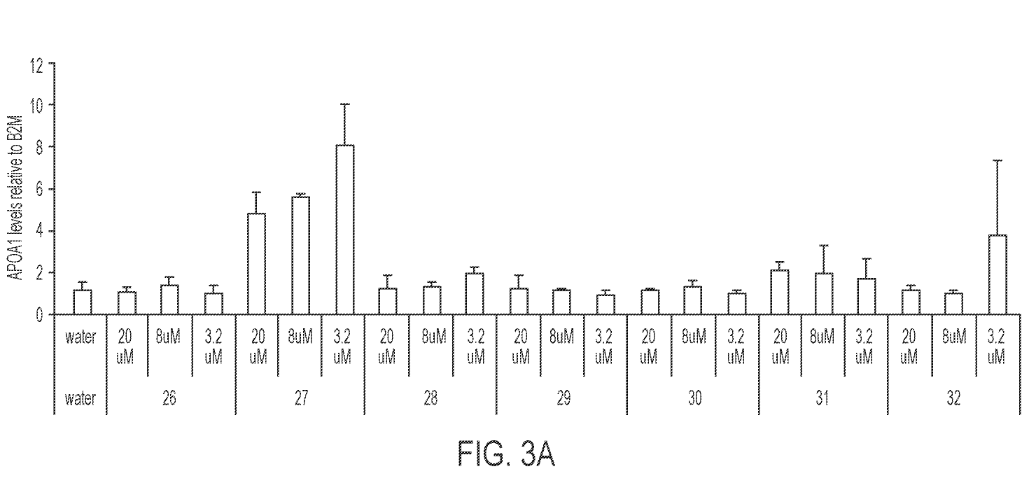

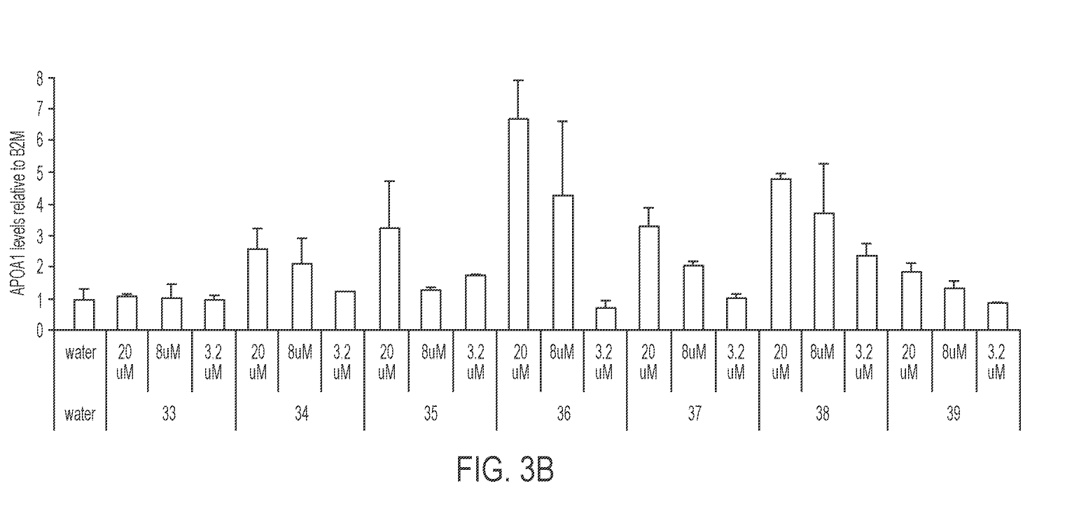

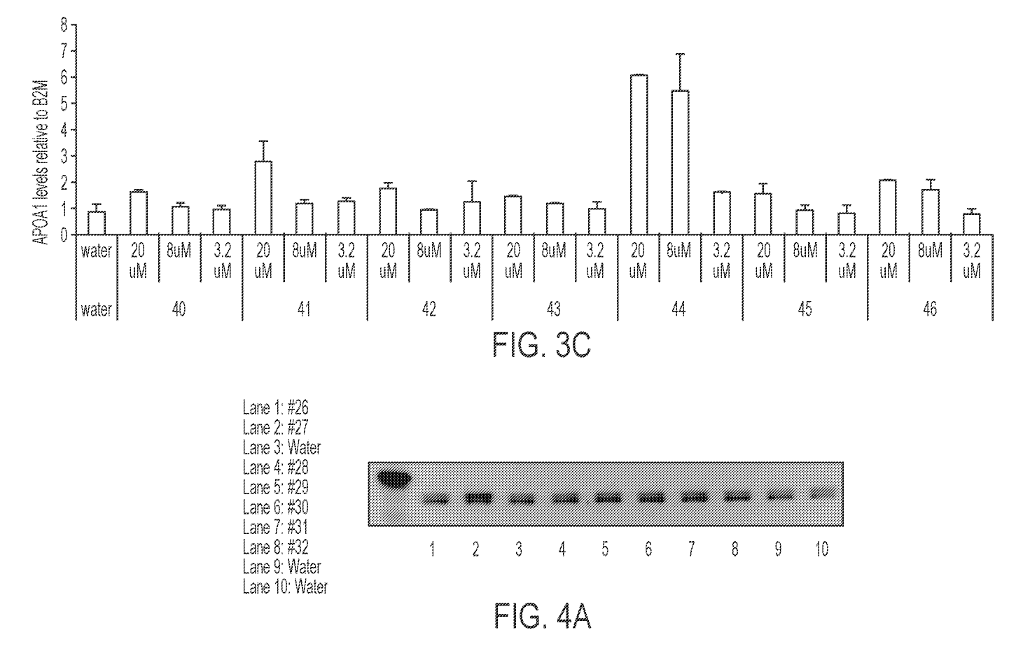

Mouse APOA1 5′ and 3′ termini lancRNA targeting oligos were screened in primary mouse hepatocytes gymnotically at 20 uM, 8 uM and 3.2 uM concentrations in duplicates. APOA1 mRNA was measured and normalized relative to the water control well and B2M housekeeper. As shown in FIGS. 3A-3C, some of the oligos tested (such as oligos Apoa1_mus-27, 34, 35, 36, 37, 38, and 44) resulted in upregulation of APOA1 mRNA levels.

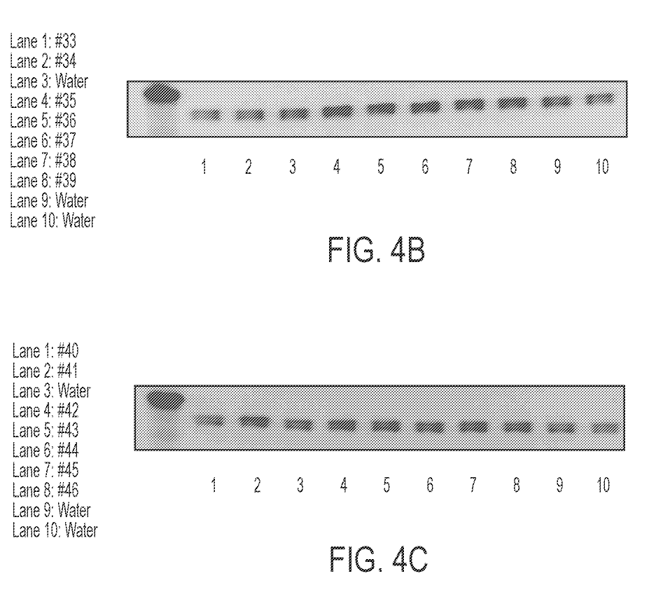

Next, mouse APOA1 5′ and 3′ termini lancRNA targeting oligos were screened in primary mouse hepatocytes gymnotically. APOA1 protein levels were measured in culture media at day5 at 8 uM oligo treatment condition. Abcam ab20453 was used as APOA1 antibody. Treatment with several oligos including Apoa1_mus-27, 35-39, and 41-45 resulted in increased APOA1 protein secretion (FIGS. 4A-4C). These results show that oligos targeting regions that encode APOA1 lancRNAs were useful for upregulation of APOA1 levels.

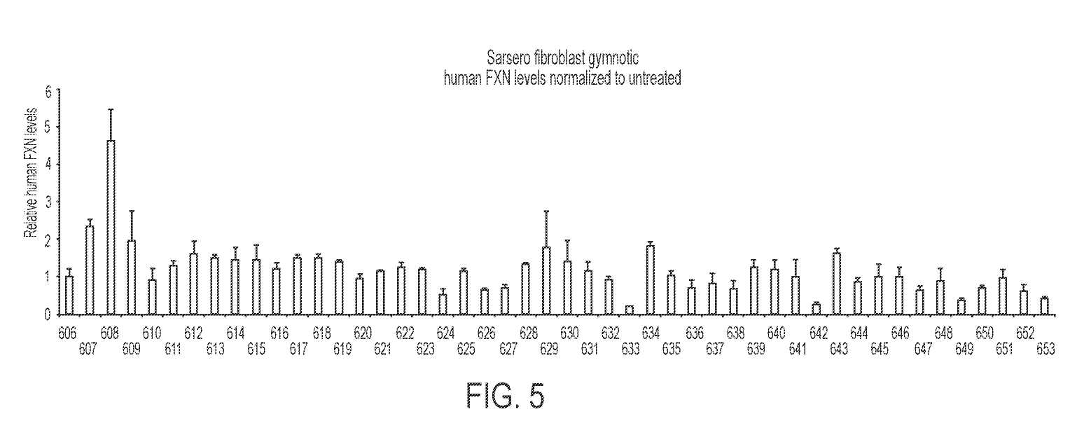

Oligos targeting FXN 3′ termini regions in antisense orientation were screened in Sarsero mouse-model derived skin fibroblasts via gymnosis and human FXN mRNA levels were measured. Oligos were screened at 10 uM concentration. Oligo and media changes were performed at day1, day4, day8. Data collection was done at day11. As shown in FIG. 5, some of the oligos tested (such as oligos FXN-607, 608, 609, 629, and 634) resulted in upregulation of FXN mRNA levels.

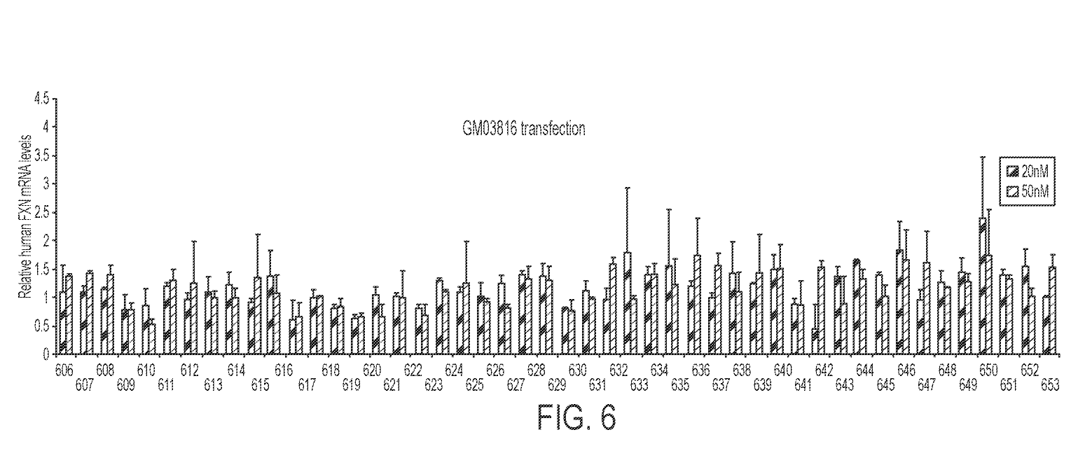

Oligos targeting FXN 3′ termini regions in antisense orientation were also screened in GM03816 cells via transfection and human FXN mRNA levels were measured. Oligos were screened at 20 nM and 50 nM concentration. Data collection was done at day4. As shown in FIG. 6, some of the oligos tested (such as oligo FXN-650) resulted in upregulation of FXN mRNA levels.

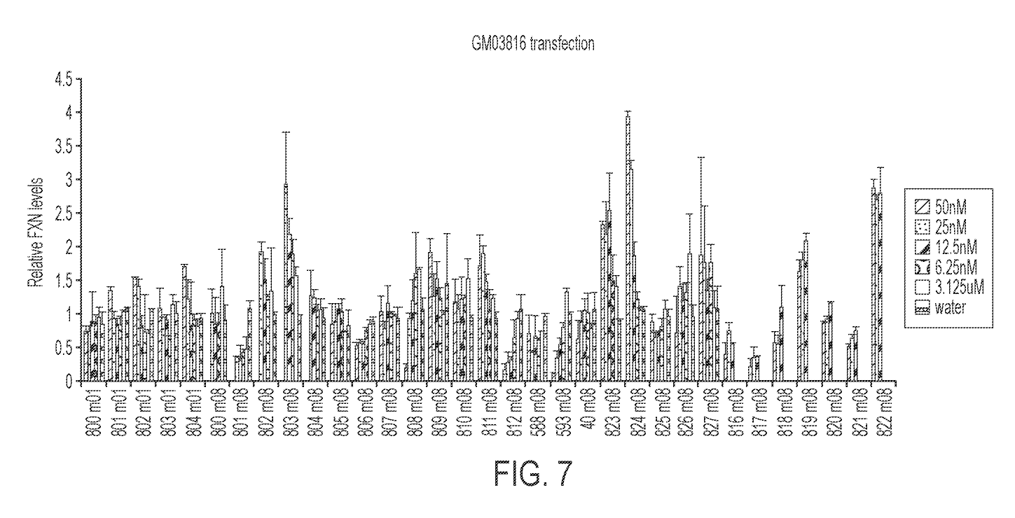

Oligos targeting FXN 5′ promoter associated regions in antisense orientation were also screened in GM03816 cells via transfection and human FXN mRNA levels were measured. Except for the oligos FXN-816 to 822, which were screened at three doses, the other oligos were screened at 5 doses. Measurements were taken at day3. As shown in FIG. 7, some of the oligos tested (such as oligos FXN-803, 823, 824, 819, and 822) resulted in upregulation of FXN mRNA levels.

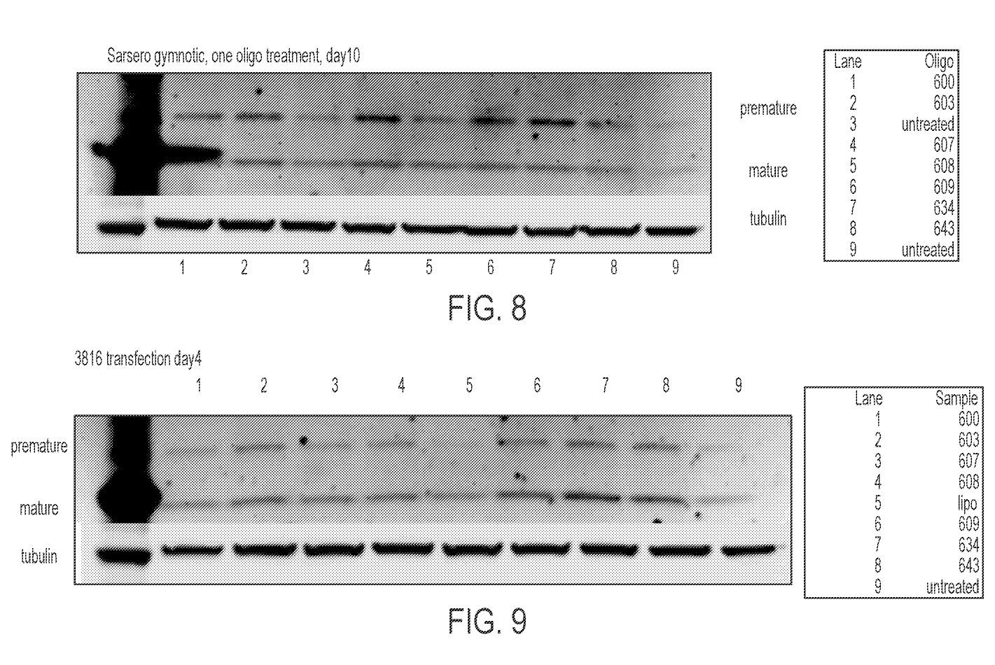

Oligos targeting FXN 3′ termini regions in antisense orientation were also screened in Sarsero mouse-model derived skin fibroblasts via gymnosis for human FXN protein levels. Measurements were taken at day10. As shown in FIG. 8, all of the oligos tested (such as oligos FXN-603, 607, 609, 634, 643) resulted in upregulation of FXN mRNA levels.

Oligos targeting FXN 3′ termini regions in antisense orientation were also screened in GM03816 cells via transfection for human FXN mRNA levels. Oligos were screened at 30 nM concentration. Data collection was done at day4. As shown in FIG. 9, all of the oligos tested (such as oligos FXN-600, 603, 607, 609, 634, 643) resulted in upregulation of FXN mRNA levels.

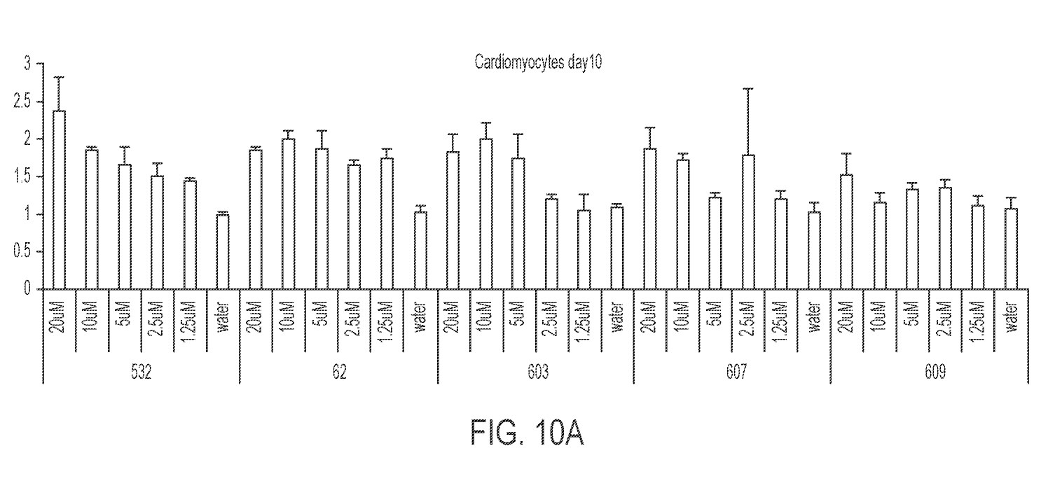

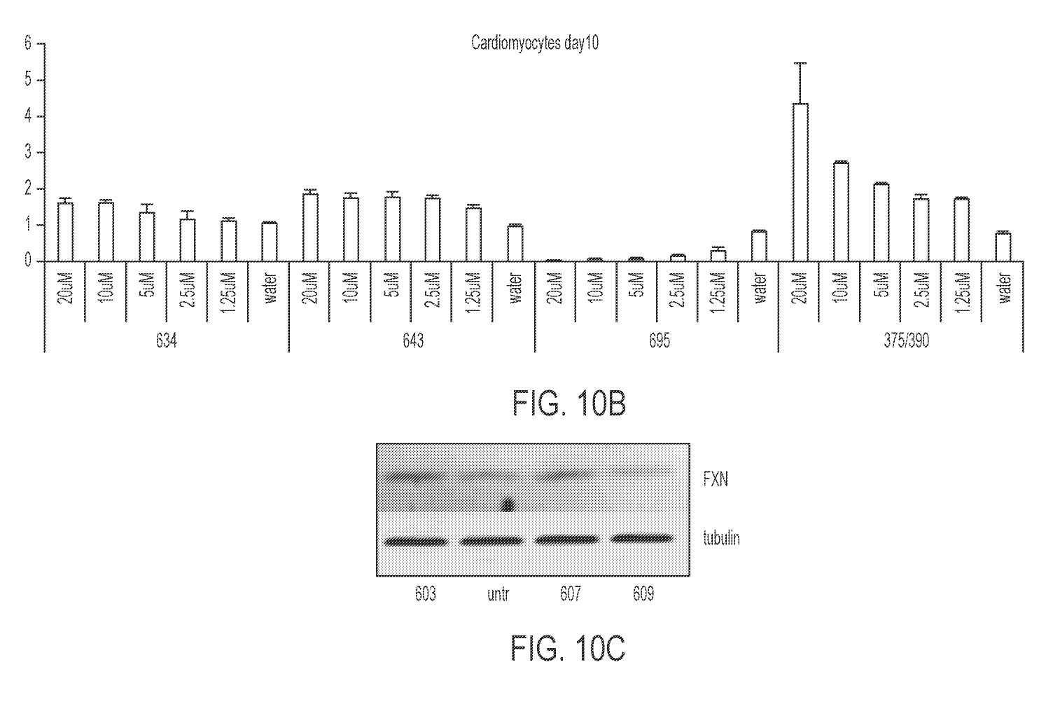

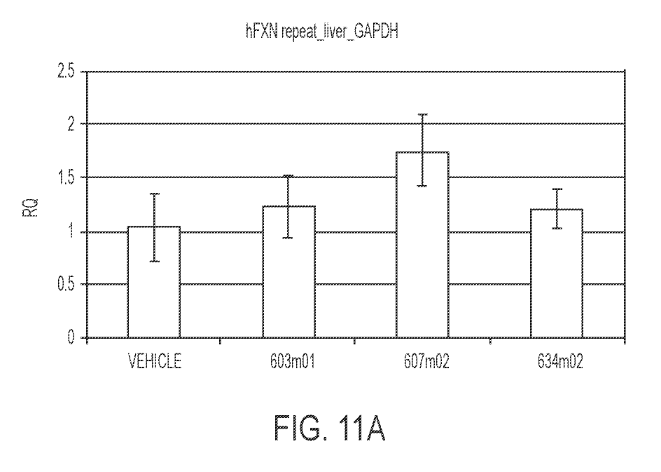

Next, screens were performed in human normal terminally differentiated cardiomyocytes. 10 uM of oligos were gymnotically delivered to human normal cardiomyocytes. Oligo treatment and media changes were done at day1, day4 and day7. Measurements for RNA and protein were taken at day10. The FXN mRNA data was normalized to GAPDH. FXN-607 showed slight FXN RNA and protein upregulation (FIGS. 10A-C). FXN-695 is a FXN gapmer and therefore downregulated FXN levels.

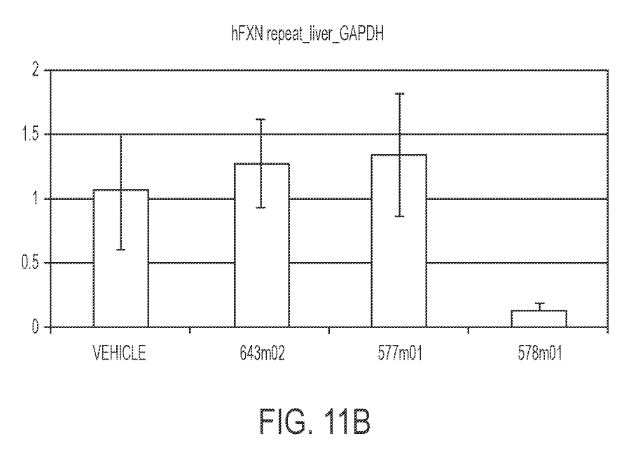

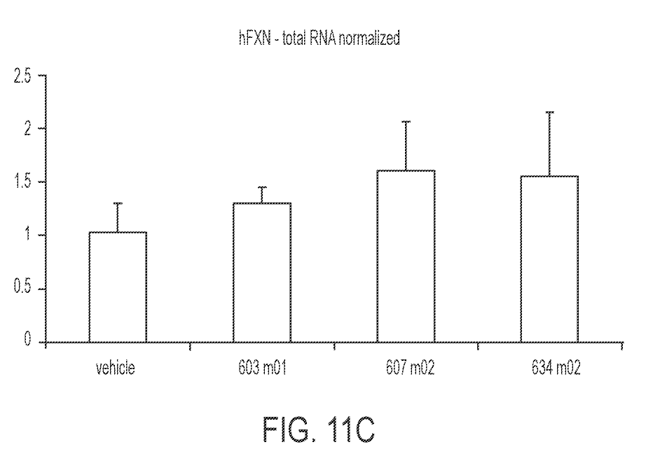

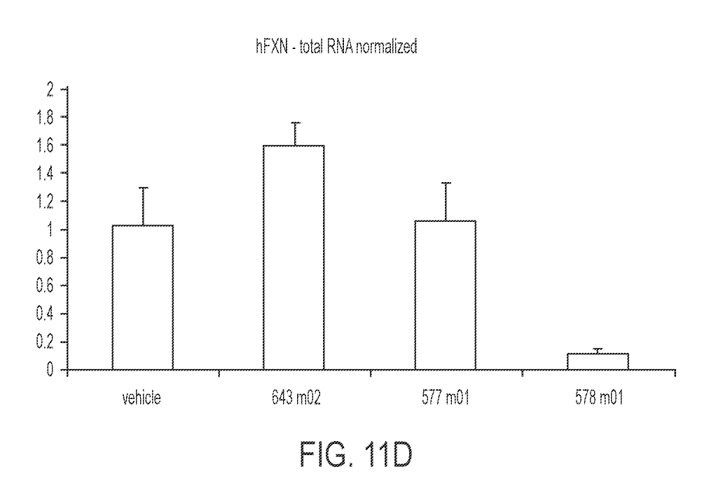

Lastly, FXN oligos were tested in vivo. Oligos were injected at 100 mg/kg at day1, day2, day3 subcutaneously to 12-16 week old Sarsero mice. Sarsero mice are an animal model of Fredreich's Ataxia. The tissue collections were done at day5. The human FXN mRNA levels were measured in liver. The data normalization was done based on GAPDH and total RNA levels. Among others, oligos 607, 634 and 643 showed human FXN upregulation in livers of Sarsero mice (FIGS. 11A-D). These results show that oligos targeting regions that encode FXN lancRNAs were useful for upregulation of FXN levels.

Together, these data show that oligos targeting regions encoding lancRNAs can be used to upregulate gene expression.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

What is claimed is:

1. A method of modulating expression of a target gene in cells, the method comprising:
delivering to the cells a single-stranded oligonucleotide of 8 to 50 nucleotides in length that comprises a region of complementarity that is complementary with at least 5 contiguous nucleotides of a low-abundance non-coding RNA (lancRNA) that modulates expression of a target gene in the cells, wherein the at least 5 contiguous nucleotides of the lancRNA are transcribed from a chromosomal region within 5 kb of a transcriptional boundary of the target gene.

2. The method of claim 1, wherein the lancRNA is represented at a level of less than 0.01 fragments per kilobase per million mapped reads (FPKM) based sequencing of RNA of the cells.

3. The method of claim 1, wherein the lancRNA is represented at an average copy number of less than 10 transcripts per cell.

4. The method of claim 3, wherein the lancRNA is represented at an average copy number of less than 0.1 transcripts per cell.

5. The method of claim 4, wherein the lancRNA is represented at an average copy number of less than 0.0001 transcripts per cell.

6. The method of claim 1, wherein the average copy number of the lancRNA is less than 1% of the average copy number of transcripts expressed from the target gene in the cells.

7. The method of any preceding claim, wherein the lancRNA is transcribed from the same strand of the chromosomal region as the target gene.

8. The method of any preceding claim, wherein the lancRNA is transcribed from the opposite strand of the chromosomal region as the target gene.

9. The method of any preceding claim, wherein the at least 5 contiguous nucleotides of the lancRNA are transcribed from a chromosomal region within 5 kb of a transcriptional boundary of the target gene.

10. The method of any preceding claim, wherein the at least 5 contiguous nucleotides of the lancRNA are transcribed from a chromosomal region within 2 kb of a transcriptional boundary of the target gene.

11. The method of any preceding claim, wherein the at least 5 contiguous nucleotides of the lancRNA are transcribed from a chromosomal region within 1 kb of a transcriptional boundary of the target gene.

12. The method of any preceding claim, wherein the at least 5 contiguous nucleotides of the lancRNA are transcribed from a chromosomal region within 500 bp of a transcriptional boundary of the target gene.

13. The method of any preceding claim, wherein the at least 5 contiguous nucleotides of the lancRNA are transcribed from a chromosomal region within 250 bp of a transcriptional boundary of the target gene.

14. The method of any preceding claim, wherein the transcriptional boundary is a transcriptional start site.

15. The method of any preceding claim, wherein the transcriptional boundary is a transcriptional end site.

16. The method of any preceding claim, wherein the lancRNA is no more than 200 nucleotides in length.

17. The method of any preceding claim, wherein the target gene is ABCA1, APOA1, ATP2A2, BDNF, FXN, HBA2, HBB, HBD, HBE1, HBG1, HBG2, SMN, UTRN, PTEN, MECP2, FOXP3, NFE2L2 (NRF2), THRB, NR1H4 (FXR), HAMP, ADIPOQ, PRKAA1, PRKAA2, PRKAB1, PRKAB2, PRKAG1, PRKAG2, or PRKAG3.

18. The method of claim 17, wherein the target gene is FXN.

19. The method of any preceding claim, wherein the oligonucleotide does not comprise three or more consecutive guanosine nucleotides.

20. The method of any preceding claim, wherein the oligonucleotide does not comprise four or more consecutive guanosine nucleotides.

21. The method of any preceding claim, wherein the oligonucleotide is 8 to 30 nucleotides in length.

22. The method of any preceding claim, wherein the oligonucleotide is 8 to 10 nucleotides in length and all but 1, 2, or 3 of the nucleotides of the complementary sequence of the lancRNA are cytosine or guanosine nucleotides.

23. The method of any preceding claim, wherein at least one nucleotide of the oligonucleotide is a nucleotide analogue.

24. The method of any preceding claim, wherein the at least one nucleotide analogue results in an increase in Tm of the oligonucleotide in a range of 1 to 5° C. compared with an oligonucleotide that does not have the at least one nucleotide analogue.

25. The method of any preceding claim, wherein at least one nucleotide of the oligonucleotide comprises a 2′ O-methyl.

26. The method of any preceding claim, wherein each nucleotide of the oligonucleotide comprises a 2′ O-methyl.

27. The method of any one of claims 1 to 26, wherein the oligonucleotide comprises at least one ribonucleotide, at least one deoxyribonucleotide, or at least one bridged nucleotide.

28. The method of claim 27, wherein the bridged nucleotide is a LNA nucleotide, a cEt nucleotide or a ENA modified nucleotide.

29. The method of any one of claims 1 to 26, wherein each nucleotide of the oligonucleotide is a LNA nucleotide.

30. The method of any one of claims 1 to 26, wherein the nucleotides of the oligonucleotide comprise alternating deoxyribonucleotides and 2′-fluoro-deoxyribonucleotides.

31. The method of any one of claims 1 to 26, wherein the nucleotides of the oligonucleotide comprise alternating deoxyribonucleotides and 2′-O-methyl nucleotides.

32. The method of any one of claims 1 to 26, wherein the nucleotides of the oligonucleotide comprise alternating deoxyribonucleotides and ENA nucleotide analogues.

33. The method of any one of claims 1 to 26, wherein the nucleotides of the oligonucleotide comprise alternating deoxyribonucleotides and LNA nucleotides.

34. The single stranded oligonucleotide of any one of claims 31 to 33, wherein the 5′ nucleotide of the oligonucleotide is a deoxyribonucleotide.

35. The method of any one of claims 1 to 26, wherein the nucleotides of the oligonucleotide comprise alternating LNA nucleotides and 2′-O-methyl nucleotides.

36. The single stranded oligonucleotide of claim 35, wherein the 5′ nucleotide of the oligonucleotide is a LNA nucleotide.

37. The method of any one of claims 1 to 26, wherein the nucleotides of the oligonucleotide comprise deoxyribonucleotides flanked by at least one LNA nucleotide on each of the 5′ and 3′ ends of the deoxyribonucleotides.

38. The method of any preceding claim, further comprising phosphorothioate internucleotide linkages between at least two nucleotides.

39. The method of claim 38, further comprising phosphorothioate internucleotide linkages between all nucleotides.

40. The method of any preceding claim, wherein the nucleotide at the 3′ position of the oligonucleotide has a 3′ hydroxyl group.

41. The method of any preceding claim, wherein the nucleotide at the 3′ position of the oligonucleotide has a 3′ thiophosphate.

42. The method of any preceding claim, further comprising a biotin moiety conjugated to the 5′ nucleotide.

43. The method of any preceding claim, wherein the single stranded oligonucleotide comprises a nucleotide sequence as set for in Table 3.

44. A single stranded oligonucleotide having a nucleotide sequence as set forth in Table 3.

45. The single stranded oligonucleotide of claim 44, wherein at least one nucleotide of the oligonucleotide comprises a 2′ O-methyl.

46. The single stranded oligonucleotide of claim 44 or 45, wherein each nucleotide of the oligonucleotide comprises a 2′ O-methyl.

47. The single stranded oligonucleotide of any one of claims 44 to 45, wherein the oligonucleotide comprises at least one ribonucleotide, at least one deoxyribonucleotide, or at least one bridged nucleotide.

48. The single strand oligonucleotide of claim 47, wherein the bridged nucleotide is a LNA nucleotide, a cEt nucleotide or a ENA modified nucleotide.

49. The single stranded oligonucleotide of any one of claims 44 to 46, wherein each nucleotide of the oligonucleotide is a LNA nucleotide.

50. The single stranded oligonucleotide of any one of claims 44 to 46, wherein the nucleotides of the oligonucleotide comprise alternating deoxyribonucleotides and 2′-fluoro-deoxyribonucleotides.

51. The single stranded oligonucleotide of any one of claims 44 to 46, wherein the nucleotides of the oligonucleotide comprise alternating deoxyribonucleotides and 2′-O-methyl nucleotides.

52. The single stranded oligonucleotide of any one of claims 44 to 46, wherein the nucleotides of the oligonucleotide comprise alternating deoxyribonucleotides and ENA nucleotide analogues.

53. The single stranded oligonucleotide of any one of claims 44 to 46, wherein the nucleotides of the oligonucleotide comprise alternating deoxyribonucleotides and LNA nucleotides.

54. The single stranded oligonucleotide of any one of claims 51 to 53, wherein the 5′ nucleotide of the oligonucleotide is a deoxyribonucleotide.

55. The single stranded oligonucleotide of any one of claims 44 to 46, wherein the nucleotides of the oligonucleotide comprise alternating LNA nucleotides and 2′-O-methyl nucleotides.

56. The single stranded oligonucleotide of claim 55, wherein the 5′ nucleotide of the oligonucleotide is a LNA nucleotide.

57. The single stranded oligonucleotide of any one of claims 44 to 46, wherein the nucleotides of the oligonucleotide comprise deoxyribonucleotides flanked by at least one LNA nucleotide on each of the 5′ and 3′ ends of the deoxyribonucleotides.

58. The single stranded oligonucleotide of any one of claims 44 to 57, further comprising phosphorothioate internucleotide linkages between at least two nucleotides.

59. The single stranded oligonucleotide of claim 58, further comprising phosphorothioate internucleotide linkages between all nucleotides.

60. The single stranded oligonucleotide of any one of claims 44 to 59, wherein the nucleotide at the 3′ position of the oligonucleotide has a 3′ hydroxyl group.

61. The single stranded oligonucleotide of any one of claims 44 to 60, wherein the nucleotide at the 3′ position of the oligonucleotide has a 3′ thiophosphate.

62. The single stranded oligonucleotide of any one of claims 44 to 61, further comprising a biotin moiety conjugated to the 5′ nucleotide.

63. The single stranded oligonucleotide of any one of claims 44 to 62, wherein the modification pattern for the oligonucleotide is the modification pattern provided in Table 3.

64. A composition comprising a single stranded oligonucleotide of any one of claims 44 to 63 and a carrier.

65. A composition comprising a single stranded oligonucleotide of any one of claims 44 to 63 in a buffered solution.

66. A composition of claim 65, wherein the oligonucleotide is conjugated to the carrier.

67. The composition of claim 66, wherein the carrier is a peptide.

68. The composition of claim 66, wherein the carrier is a steroid.

69. A pharmaceutical composition comprising a composition of any one of claims 61 to 65 and a pharmaceutically acceptable carrier.

70. A kit comprising a container housing the composition of any one of claims 64 to 69.

71. A method of modulating expression of a target gene in cells, the method comprising:
i) determining presence of a low-abundance non-coding RNA (lancRNA) in cells; and
ii) based on the determination made in i), delivering to the cells a single-stranded oligonucleotide of 8 to 50 nucleotides in length that comprises a region of complementarity that is complementary with at least 5 contiguous nucleotides of a lancRNA that modulates expression of a target gene in the cells, wherein the at least 5 contiguous nucleotides of the lancRNA are transcribed from a chromosomal region within 5 kb of a transcriptional boundary of the target gene.

72. The method of claim 71, wherein in step i) the lancRNA is determined to be present at a level of less than 0.01 fragments per kilobase per million mapped reads (FPKM) based sequencing of RNA of the cells.

73. The method of claim 71, wherein in step i) the lancRNA is determined to be present at an average copy number of less than 10 transcripts per cell.

74. The method of claim 73, wherein in step i) the lancRNA is determined to be present at an average copy number of less than 0.1 transcripts per cell.

75. The method of claim 74, wherein in step i) the lancRNA is determined to be present at an average copy number of less than 0.0001 transcripts per cell.

76. The method of claim 71, wherein in step i) the lancRNA is determined to be present at less than 1% of the average copy number of transcripts expressed from the target gene in the cells.

77. A method of modulating expression of a target gene in cells, the method comprising:
delivering to the cells a single-stranded oligonucleotide of 8 to 50 nucleotides in length that comprises a region of complementarity that is complementary with at least 5 contiguous nucleotides of a chromosomal region that corresponds to a 3′ UTR of the target gene, wherein the at least 5 contiguous nucleotides are antisense to the target gene.

78. The method of claim 77, wherein the target gene is ABCA1, APOA1, ATP2A2, BDNF, FXN, HBA2, HBB, HBD, HBE1, HBG1, HBG2, SMN, UTRN, PTEN, MECP2, FOXP3, NFE2L2 (NRF2), THRB, NR1H4 (FXR), HAMP, ADIPOQ, PRKAA1, PRKAA2, PRKAB1, PRKAB2, PRKAG1, PRKAG2, or PRKAG3.

79. The method of claim 78, wherein the target gene is FXN.

80. The method of any one of claims 77-79, wherein the oligonucleotide does not comprise three or more consecutive guanosine nucleotides.

81. The method of any one of claims 77-81, wherein the oligonucleotide does not comprise four or more consecutive guanosine nucleotides.

82. The method of any one of claims 77-81, wherein the oligonucleotide is 8 to 30 nucleotides in length.

83. The method of any one of claims 77-82, wherein the oligonucleotide is 8 to 10 nucleotides in length and all but 1, 2, or 3 of the nucleotides of the complementary sequence of the lancRNA are cytosine or guanosine nucleotides.

84. The method of any one of claims 77-83, wherein at least one nucleotide of the oligonucleotide is a nucleotide analogue.

85. The method of any one of claims 77-84, wherein the at least one nucleotide analogue results in an increase in Tm of the oligonucleotide in a range of 1 to 5° C. compared with an oligonucleotide that does not have the at least one nucleotide analogue.

86. The method of any one of claims 77-85, wherein at least one nucleotide of the oligonucleotide comprises a 2′ O-methyl.

87. The method of any one of claims 77-86, wherein the oligonucleotide comprises at least one ribonucleotide, at least one deoxyribonucleotide, or at least one bridged nucleotide.

88. The method of claim 87, wherein the bridged nucleotide is a LNA nucleotide, a cEt nucleotide or a ENA modified nucleotide.

89. The method of any one of claims 77-88, further comprising phosphorothioate internucleotide linkages between at least two nucleotides.

90. The single stranded oligonucleotide of claim 89, further comprising phosphorothioate internucleotide linkages between all nucleotides.

91. The method of any one of claims 77-90, wherein the nucleotide at the 3′ position of the oligonucleotide has a 3′ hydroxyl group.

92. The method of any one of claims 77-90, wherein the nucleotide at the 3′ position of the oligonucleotide has a 3′ thiophosphate.


SEQUENCE LISTING (296 sequences)

SEQ ID NO: 1 - DNA, 15 bp, Homo sapiens
cgcagtagccggcct

SEQ ID NO: 2 - DNA, 15 bp, Homo sapiens
cctggcgtcacccag

SEQ ID NO: 3 - DNA, 15 bp, Homo sapiens
cccagcccaggccca

SEQ ID NO: 4 - DNA, 15 bp, Homo sapiens
cccagaccctcaccc

SEQ ID NO: 5 - DNA, 15 bp, Homo sapiens
tcccgcggccggcag

SEQ ID NO: 6 - DNA, 15 bp, Homo sapiens
agagttggccccact

SEQ ID NO: 7 - DNA, 15 bp, Homo sapiens
agcagctggtcaacc

SEQ ID NO: 8 - DNA, 15 bp, Homo sapiens
tgctcactgttctat

SEQ ID NO: 9 - DNA, 15 bp, Homo sapiens
ctccaaatgagacac

SEQ ID NO: 10 - DNA, 15 bp, Homo sapiens
attaaagggtagcct

SEQ ID NO: 11 - DNA, 15 bp, Homo sapiens
acaaatgttttcagg

SEQ ID NO: 12 - DNA, 15 bp, Homo sapiens
cttctttcaaagtgt

SEQ ID NO: 13 - DNA, 15 bp, Homo sapiens
taatgtcttatgcct

SEQ ID NO: 14 - DNA, 15 bp, Homo sapiens
atacctgaatataac

SEQ ID NO: 15 - DNA, 15 bp, Homo sapiens
aacctttaaaaaagc

SEQ ID NO: 16 - DNA, 15 bp, Homo sapiens
aaaataataagaagg

SEQ ID NO: 17 - DNA, 15 bp, Homo sapiens
aaaaattccaggagg

SEQ ID NO: 18 - DNA, 15 bp, Homo sapiens
gaaaatgaattgtct

SEQ ID NO: 19 - DNA, 15 bp, Homo sapiens
tcactcttcattctt

SEQ ID NO: 20 - DNA, 15 bp, Homo sapiens
tgaaggatttactgc

SEQ ID NO: 21 - DNA, 14 bp, Homo sapiens
ttggattgtcggat

SEQ ID NO: 22 - DNA, 15 bp, Homo sapiens
ttcctccctcacatg

SEQ ID NO: 23 - DNA, 15 bp, Homo sapiens
ataccccttatcttt

SEQ ID NO: 24 - DNA, 15 bp, Homo sapiens
tataatgtcttatgc

SEQ ID NO: 25 - DNA, 15 bp, Homo sapiens
tgaggacagttgggc

SEQ ID NO: 26 - DNA, 15 bp, Homo sapiens
tatgtgtcacagctc

SEQ ID NO: 27 - DNA, 15 bp, Homo sapiens
tgtagaaagaatgtg

SEQ ID NO: 28 - DNA, 15 bp, Homo sapiens
ttgcctcctaccttg

SEQ ID NO: 29 - DNA, 15 bp, Homo sapiens
cccccaagttctgat

SEQ ID NO: 30 - DNA, 15 bp, Homo sapiens
atgcttgatgcccag

SEQ ID NO: 31 - DNA, 15 bp, Homo sapiens
ccccgttttaaggac

SEQ ID NO: 32 - DNA, 15 bp, Homo sapiens
attaaaagctatcag

SEQ ID NO: 33 - DNA, 15 bp, Homo sapiens
gccaagaccccagct

SEQ ID NO: 34 - DNA, 15 bp, Homo sapiens
tcattatgcagctga

SEQ ID NO: 35 - DNA, 15 bp, Homo sapiens
ggtctgttttttgtt

SEQ ID NO: 36 - DNA, 15 bp, Homo sapiens
gttgttgttgtttat

SEQ ID NO: 37 - DNA, 15 bp, Homo sapiens
gcagagctcactaaa

SEQ ID NO: 38 - DNA, 15 bp, Homo sapiens
gccttaaaaaccaaa

SEQ ID NO: 39 - DNA, 15 bp, Homo sapiens
ctggacttgtcttcc

SEQ ID NO: 40 - DNA, 15 bp, Homo sapiens
caggacattaaaatt

SEQ ID NO: 41 - DNA, 15 bp, Homo sapiens
catgcaaagttatgc

SEQ ID NO: 42 - DNA, 15 bp, Homo sapiens
aagtgcagtaggcca

SEQ ID NO: 43 - DNA, 15 bp, Homo sapiens
gtgccagtgagaaaa

SEQ ID NO: 44 - DNA, 15 bp, Homo sapiens
taaataacatcatac

SEQ ID NO: 45 - DNA, 15 bp, Homo sapiens
atgtttgtatgtgtt

SEQ ID NO: 46 - DNA, 15 bp, Homo sapiens
aatctataaaatgga

SEQ ID NO: 47 - DNA, 15 bp, Homo sapiens
cagtttgcattaaat

SEQ ID NO: 48 - DNA, 15 bp, Homo sapiens
tataggtttacaata

SEQ ID NO: 49 - DNA, 15 bp, Homo sapiens
gttataattatatgt

SEQ ID NO: 50 - DNA, 15 bp, Homo sapiens
ttaagatagttgttc

SEQ ID NO: 51 - DNA, 15 bp, Homo sapiens
aataaactctaaata

SEQ ID NO: 52 - DNA, 15 bp, Homo sapiens
accccaactccaaga

SEQ ID NO: 53 - DNA, 15 bp, Homo sapiens
gtgttagcaagaaat

SEQ ID NO: 54 - DNA, 15 bp, Mus musculus
tccaaaatggaatag

SEQ ID NO: 55 - DNA, 15 bp, Mus musculus
tttccaaaatggaat

SEQ ID NO: 56 - DNA, 15 bp, Mus musculus
acctttccaaaatgg

SEQ ID NO: 57 - DNA, 15 bp, Mus musculus
aacctttccaaaatg

SEQ ID NO: 58 - DNA, 15 bp, Mus musculus
acaataaacctttcc

SEQ ID NO: 59 - DNA, 15 bp, Mus musculus
ggtgcccgcttccac

SEQ ID NO: 60 - DNA, 15 bp, Mus musculus
tccactccccacccc

SEQ ID NO: 61 - DNA, 15 bp, Mus musculus
acccccgcattggct

SEQ ID NO: 62 - DNA, 15 bp, Mus musculus
tggctttcttacaat

SEQ ID NO: 63 - DNA, 15 bp, Mus musculus
ggaatagcttctttc

SEQ ID NO: 64 - DNA, 15 bp, Mus musculus
ctttctttgggggac

SEQ ID NO: 65 - DNA, 15 bp, Mus musculus
cacccagactgtcgg

SEQ ID NO: 66 - DNA, 15 bp, Mus musculus
cagggccaggctgag

SEQ ID NO: 67 - DNA, 15 bp, Mus musculus
gctgatccttgaact

SEQ ID NO: 68 - DNA, 15 bp, Mus musculus
agactgtcggagagc

SEQ ID NO: 69 - DNA, 15 bp, Mus musculus
tgtcggagagctccg

SEQ ID NO: 70 - DNA, 15 bp, Mus musculus
gctggacacccagac

SEQ ID NO: 71 - DNA, 15 bp, Mus musculus
gaagagctggacacc

SEQ ID NO: 72 - DNA, 15 bp, Mus musculus
gggaagaagagctgg

SEQ ID NO: 73 - DNA, 15 bp, Mus musculus
gaccagggaagaaga

SEQ ID NO: 74 - DNA, 15 bp, Mus musculus
cacatatatagacca

SEQ ID NO: 75 - DNA, 15 bp, Homo sapiens
gtctcccttgggtca

SEQ ID NO: 76 - DNA, 15 bp, Homo sapiens
tgcggccagtggcca

SEQ ID NO: 77 - DNA, 15 bp, Homo sapiens
caccaggggtcgccg

SEQ ID NO: 78 - DNA, 15 bp, Homo sapiens
cagcgctggagggcg

SEQ ID NO: 79 - DNA, 15 bp, Homo sapiens
ctggagggcggagcg

SEQ ID NO: 80 - DNA, 15 bp, Homo sapiens
gtctcccttgggtca

SEQ ID NO: 81 - DNA, 15 bp, Homo sapiens
tgcggccagtggcca

SEQ ID NO: 82 - DNA, 15 bp, Homo sapiens
caccaggggtcgccg

SEQ ID NO: 83 - DNA, 15 bp, Homo sapiens
cagcgctggagggcg

SEQ ID NO: 84 - DNA, 15 bp, Homo sapiens
ctggagggcggagcg

SEQ ID NO: 85 - DNA, 15 bp, Homo sapiens
aactgctgtaaaccc

SEQ ID NO: 86 - DNA, 15 bp, Homo sapiens
ataccggcggccaag

SEQ ID NO: 87 - DNA, 15 bp, Homo sapiens
cagcctcaatttgtg

SEQ ID NO: 88 - DNA, 15 bp, Homo sapiens
catgcacccacttcc

SEQ ID NO: 89 - DNA, 15 bp, Homo sapiens
cagcaagacagcagc

SEQ ID NO: 90 - DNA, 15 bp, Homo sapiens
tcccaagttcctcct

SEQ ID NO: 91 - DNA, 15 bp, Homo sapiens
gtttagaattttaga

SEQ ID NO: 92 - DNA, 15 bp, Homo sapiens
ggctgcagtctccct

SEQ ID NO: 93 - DNA, 15 bp, Homo sapiens
tcctggttgcactcc

SEQ ID NO: 94 - DNA, 15 bp, Homo sapiens
agttcttcctgaggt

SEQ ID NO: 95 - DNA, 15 bp, Homo sapiens
ctaacctctagctgc

SEQ ID NO: 96 - DNA, 15 bp, Homo sapiens
cacagaagagtgcct

SEQ ID NO: 97 - DNA, 15 bp, Homo sapiens
gccagtggccaccag

SEQ ID NO: 98 - DNA, 15 bp, Homo sapiens
gcagcacccagcgct

SEQ ID NO: 99 - DNA, 15 bp, Homo sapiens
gagcagcatgtggac

SEQ ID NO: 100 - DNA, 15 bp, Homo sapiens
tctcccactcaacac

SEQ ID NO: 101 - DNA, 15 bp, Homo sapiens
tcacacctgttagtt

SEQ ID NO: 102 - DNA, 15 bp, Homo sapiens
ttcctcttgacactt

SEQ ID NO: 103 - DNA, 15 bp, Homo sapiens
gtcatttagcatcct

SEQ ID NO: 104 - DNA, 15 bp, Homo sapiens
aagtatgtaaacatg

SEQ ID NO: 105 - DNA, 15 bp, Homo sapiens
cacgattcacaaagt

SEQ ID NO: 106 - DNA, 15 bp, Homo sapiens
ggctttggaagaact

SEQ ID NO: 107 - DNA, 15 bp, Homo sapiens
ttagtaccttcccat

SEQ ID NO: 108 - DNA, 15 bp, Homo sapiens
gtctcccttgggtca

SEQ ID NO: 109 - DNA, 15 bp, Homo sapiens
cgctccgccctccag

SEQ ID NO: 110 - DNA, 15 bp, Homo sapiens
attattttgcttttt

SEQ ID NO: 111 - DNA, 17 bp, Homo sapiens
aggccacggcggccgca

SEQ ID NO: 112 - DNA, 17 bp, Homo sapiens
catcgatgtcggtgcgc

SEQ ID NO: 113 - DNA, 15 bp, Homo sapiens
acacatagcccaact

SEQ ID NO: 114 - DNA, 15 bp, Homo sapiens
ctcagaccaccgcct

SEQ ID NO: 115 - DNA, 15 bp, Homo sapiens
ccaccgcctcccctg

SEQ ID NO: 116 - DNA, 15 bp, Homo sapiens
caggccccataaaag

SEQ ID NO: 117 - DNA, 15 bp, Homo sapiens
cataaaagcgactgt

SEQ ID NO: 118 - DNA, 15 bp, Homo sapiens
actgtcactcggtcc

SEQ ID NO: 119 - DNA, 15 bp, Homo sapiens
actcggtcccagaca

SEQ ID NO: 120 - DNA, 15 bp, Homo sapiens
agacaccagagcaag

SEQ ID NO: 121 - DNA, 15 bp, Homo sapiens
gagcaagctcaagac

SEQ ID NO: 122 - DNA, 15 bp, Homo sapiens
gcaagacgtagaacc

SEQ ID NO: 123 - DNA, 15 bp, Homo sapiens
agacgtagaacctac

SEQ ID NO: 124 - DNA, 15 bp, Homo sapiens
agaacctacctgccc

SEQ ID NO: 125 - DNA, 15 bp, Homo sapiens
acataggtcttggaa

SEQ ID NO: 126 - DNA, 15 bp, Homo sapiens
aggtcttggaataaa

SEQ ID NO: 127 - DNA, 15 bp, Homo sapiens
tggaataaaatggct

SEQ ID NO: 128 - DNA, 15 bp, Homo sapiens
tggctggttcttttg

SEQ ID NO: 129 - DNA, 15 bp, Homo sapiens
gttttccaaaccaga

SEQ ID NO: 130 - DNA, 15 bp, Homo sapiens
accagagtgtctgtt

SEQ ID NO: 131 - DNA, 15 bp, Homo sapiens
agtgtctgttgtcct

SEQ ID NO: 132 - DNA, 15 bp, Homo sapiens
gttgtcctttctctc

SEQ ID NO: 133 - DNA, 15 bp, Homo sapiens
ctttctctctgccga

SEQ ID NO: 134 - DNA, 15 bp, Homo sapiens
ctgccgagtgtctgt

SEQ ID NO: 135 - DNA, 15 bp, Homo sapiens
ctcagaccaccgcct

SEQ ID NO: 136 - DNA, 15 bp, Homo sapiens
ccaccgcctcccctg

SEQ ID NO: 137 - DNA, 15 bp, Homo sapiens
caggccccataaaag

SEQ ID NO: 138 - DNA, 15 bp, Homo sapiens
cataaaagcgactgt

SEQ ID NO: 139 - DNA, 15 bp, Homo sapiens
actgtcactcggtcc

SEQ ID NO: 140 - DNA, 15 bp, Homo sapiens
actcggtcccagaca

SEQ ID NO: 141 - DNA, 15 bp, Homo sapiens
agacaccagagcaag

SEQ ID NO: 142 - DNA, 15 bp, Homo sapiens
gagcaagctcaagac

SEQ ID NO: 143 - DNA, 15 bp, Homo sapiens
gcaagacgtagaacc

SEQ ID NO: 144 - DNA, 15 bp, Homo sapiens
agacgtagaacctac

SEQ ID NO: 145 - DNA, 15 bp, Homo sapiens
agaacctacctgccc

SEQ ID NO: 146 - DNA, 15 bp, Homo sapiens
acataggtcttggaa

SEQ ID NO: 147 - DNA, 15 bp, Homo sapiens
aggtcttggaataaa

SEQ ID NO: 148 - DNA, 15 bp, Homo sapiens
tggaataaaatggct

SEQ ID NO: 149 - DNA, 15 bp, Homo sapiens
tggctggttcttttg

SEQ ID NO: 150 - DNA, 15 bp, Homo sapiens
gttttccaaaccaga

SEQ ID NO: 151 - DNA, 15 bp, Homo sapiens
accagagtgtctgtt

SEQ ID NO: 152 - DNA, 15 bp, Homo sapiens
agtgtctgttgtcct

SEQ ID NO: 153 - DNA, 15 bp, Homo sapiens
gttgtcctttctctc

SEQ ID NO: 154 - DNA, 15 bp, Homo sapiens
ctttctctctgccga

SEQ ID NO: 155 - DNA, 15 bp, Homo sapiens
ctgccgagtgtctgt

SEQ ID NO: 156 - DNA, 15 bp, Homo sapiens
ctctcccaaggttcc

SEQ ID NO: 157 - DNA, 15 bp, Homo sapiens
aggttcctttctatg

SEQ ID NO: 158 - DNA, 15 bp, Homo sapiens
atgtttatatcattt

SEQ ID NO: 159 - DNA, 15 bp, Homo sapiens
atatcatttagcagg

SEQ ID NO: 160 - DNA, 15 bp, Homo sapiens
attgttaatgactaa

SEQ ID NO: 161 - DNA, 15 bp, Homo sapiens
agcttctagttcagt

SEQ ID NO: 162 - DNA, 15 bp, Homo sapiens
agtgatagagctatt

SEQ ID NO: 163 - DNA, 15 bp, Homo sapiens
agagagggaagatga

SEQ ID NO: 164 - DNA, 15 bp, Homo sapiens
agttgatgtgtacag

SEQ ID NO: 165 - DNA, 15 bp, Homo sapiens
acgggtgcccaggag

SEQ ID NO: 166 - DNA, 15 bp, Homo sapiens
cacaaaacggccaga

SEQ ID NO: 167 - DNA, 15 bp, Homo sapiens
atattgcatatattt

SEQ ID NO: 168 - DNA, 15 bp, Homo sapiens
atattttattaaaga

SEQ ID NO: 169 - DNA, 15 bp, Homo sapiens
agagttgtattcaat

SEQ ID NO: 170 - DNA, 15 bp, Homo sapiens
tgtattcaatcttgg

SEQ ID NO: 171 - DNA, 15 bp, Homo sapiens
caatcttggcaataa

SEQ ID NO: 172 - DNA, 15 bp, Homo sapiens
agcaaacataatggc

SEQ ID NO: 173 - DNA, 15 bp, Homo sapiens
atggcaacaggattt

SEQ ID NO: 174 - DNA, 15 bp, Homo sapiens
ttttctttgggaaca

SEQ ID NO: 175 - DNA, 15 bp, Homo sapiens
attctaattggcaag

SEQ ID NO: 176 - DNA, 15 bp, Homo sapiens
attggcaagccctgt

SEQ ID NO: 177 - DNA, 15 bp, Homo sapiens
agccctgtttgccta

SEQ ID NO: 178 - DNA, 15 bp, Homo sapiens
ctaattaaattgatt

SEQ ID NO: 179 - DNA, 15 bp, Homo sapiens
attgttacttcaatt

SEQ ID NO: 180 - DNA, 15 bp, Homo sapiens
ttctatctgttgaac

SEQ ID NO: 181 - DNA, 15 bp, Homo sapiens
ctctcccaaggttcc

SEQ ID NO: 182 - DNA, 15 bp, Homo sapiens
aggttcctttctatg

SEQ ID NO: 183 - DNA, 15 bp, Homo sapiens
atgtttatatcattt

SEQ ID NO: 184 - DNA, 15 bp, Homo sapiens
atatcatttagcagg

SEQ ID NO: 185 - DNA, 15 bp, Homo sapiens
attgttaatgactaa

SEQ ID NO: 186 - DNA, 15 bp, Homo sapiens
agcttctagttcagt

SEQ ID NO: 187 - DNA, 15 bp, Homo sapiens
agtgatagagctatt

SEQ ID NO: 188 - DNA, 15 bp, Homo sapiens
agagagggaagatga

SEQ ID NO: 189 - DNA, 15 bp, Homo sapiens
agttgatgtgtacag

SEQ ID NO: 190 - DNA, 15 bp, Homo sapiens
acgggtgcccaggag

SEQ ID NO: 191 - DNA, 15 bp, Homo sapiens
cacaaaacggccaga

SEQ ID NO: 192 - DNA, 15 bp, Homo sapiens
atattgcatatattt

SEQ ID NO: 193 - DNA, 15 bp, Homo sapiens
atattttattaaaga

SEQ ID NO: 194 - DNA, 15 bp, Homo sapiens
agagttgtattcaat

SEQ ID NO: 195 - DNA, 15 bp, Homo sapiens
tgtattcaatcttgg

SEQ ID NO: 196 - DNA, 15 bp, Homo sapiens
caatcttggcaataa

SEQ ID NO: 197 - DNA, 15 bp, Homo sapiens
agcaaacataatggc

SEQ ID NO: 198 - DNA, 15 bp, Homo sapiens
atggcaacaggattt

SEQ ID NO: 199 - DNA, 15 bp, Homo sapiens
ttttctttgggaaca

SEQ ID NO: 200 - DNA, 15 bp, Homo sapiens
attctaattggcaag

SEQ ID NO: 201 - DNA, 15 bp, Homo sapiens
attggcaagccctgt

SEQ ID NO: 202 - DNA, 15 bp, Homo sapiens
agccctgtttgccta

SEQ ID NO: 203 - DNA, 15 bp, Homo sapiens
ctaattaaattgatt

SEQ ID NO: 204 - DNA, 15 bp, Homo sapiens
attgttacttcaatt

SEQ ID NO: 205 - DNA, 15 bp, Homo sapiens
ttctatctgttgaac

SEQ ID NO: 206 - DNA, 15 bp, Homo sapiens
gaatatagtgggcgt

SEQ ID NO: 207 - DNA, 15 bp, Homo sapiens
agtgggcgtagataa

SEQ ID NO: 208 - DNA, 15 bp, Homo sapiens
cgtagataaactcat

SEQ ID NO: 209 - DNA, 15 bp, Homo sapiens
taaactcataagctt

SEQ ID NO: 210 - DNA, 15 bp, Homo sapiens
cataagcttaaattc

SEQ ID NO: 211 - DNA, 15 bp, Homo sapiens
aagcttataacagat

SEQ ID NO: 212 - DNA, 15 bp, Homo sapiens
ataacagatatattt

SEQ ID NO: 213 - DNA, 15 bp, Homo sapiens
gatatattttcctgt

SEQ ID NO: 214 - DNA, 15 bp, Homo sapiens
tttcctgtctctttc

SEQ ID NO: 215 - DNA, 15 bp, Homo sapiens
atggatttttacata

SEQ ID NO: 216 - DNA, 15 bp, Homo sapiens
tgtatgcagatataa

SEQ ID NO: 217 - DNA, 15 bp, Homo sapiens
ctgtaattatgaata

SEQ ID NO: 218 - DNA, 15 bp, Homo sapiens
tacataggcaaagag

SEQ ID NO: 219 - DNA, 15 bp, Homo sapiens
caaagagttgcctgc

SEQ ID NO: 220 - DNA, 15 bp, Homo sapiens
ccagccgcttcctgc

SEQ ID NO: 221 - DNA, 15 bp, Homo sapiens
tagacatggatgaaa

SEQ ID NO: 222 - DNA, 15 bp, Homo sapiens
gatgaaattgcccct

SEQ ID NO: 223 - DNA, 15 bp, Homo sapiens
tgccccttgaatgcg

SEQ ID NO: 224 - DNA, 15 bp, Homo sapiens
tgaatgcgggtactt

SEQ ID NO: 225 - DNA, 15 bp, Homo sapiens
gtacttgaaactatt

SEQ ID NO: 226 - DNA, 15 bp, Homo sapiens
actattgcatttcgt

SEQ ID NO: 227 - DNA, 15 bp, Homo sapiens
cgttctccggtcctg

SEQ ID NO: 228 - DNA, 15 bp, Homo sapiens
ctgtgatgtgaatgc

SEQ ID NO: 229 - DNA, 15 bp, Homo sapiens
gttcgaggattagac

SEQ ID NO: 230 - DNA, 15 bp, Homo sapiens
attagactgactgga

SEQ ID NO: 231 - DNA, 15 bp, Homo sapiens
actggattcattctc

SEQ ID NO: 232 - DNA, 15 bp, Homo sapiens
attctcataattcct

SEQ ID NO: 233 - DNA, 15 bp, Homo sapiens
attcctacagcacta

SEQ ID NO: 234 - DNA, 15 bp, Homo sapiens
tcatttcattccatt

SEQ ID NO: 235 - DNA, 15 bp, Homo sapiens
tccattgcctagctc

SEQ ID NO: 236 - DNA, 15 bp, Homo sapiens
accaggtcaccggtt

SEQ ID NO: 237 - DNA, 15 bp, Homo sapiens
cgcagtagcttccta

SEQ ID NO: 238 - DNA, 15 bp, Homo sapiens
caaggagttgacatt

SEQ ID NO: 239 - DNA, 15 bp, Homo sapiens
acattttgcaggact

SEQ ID NO: 240 - DNA, 15 bp, Homo sapiens
caaggaaggcgcaca

SEQ ID NO: 241 - DNA, 15 bp, Homo sapiens
attaactttgcatga

SEQ ID NO: 242 - DNA, 15 bp, Homo sapiens
tgaataatgtgagtg

SEQ ID NO: 243 - DNA, 15 bp, Homo sapiens
gtaatttggctagag

SEQ ID NO: 244 - DNA, 15 bp, Homo sapiens
acagttccaactgtc

SEQ ID NO: 245 - DNA, 15 bp, Homo sapiens
atcactctgaacatt

SEQ ID NO: 246 - DNA, 15 bp, Homo sapiens
gagcctatattcata

SEQ ID NO: 247 - DNA, 15 bp, Homo sapiens
atgcatttaggtcta

SEQ ID NO: 248 - DNA, 15 bp, Homo sapiens
atgctgtgatagagt

SEQ ID NO: 249 - DNA, 15 bp, Homo sapiens
catattaatgcattt

SEQ ID NO: 250 - DNA, 15 bp, Homo sapiens
tcatcagcctgatta

SEQ ID NO: 251 - DNA, 15 bp, Homo sapiens
tacggagtggacagt

SEQ ID NO: 252 - DNA, 15 bp, Homo sapiens
caatcgcagcggctc

SEQ ID NO: 253 - DNA, 15 bp, Homo sapiens
cagctgttgacatgt

SEQ ID NO: 254 - DNA, 15 bp, Homo sapiens
atggagtttggcatc

SEQ ID NO: 255 - DNA, 15 bp, Homo sapiens
catgatgaggaagtt

SEQ ID NO: 256 - DNA, 15 bp, Homo sapiens
ctctgttcctcaaac

SEQ ID NO: 257 - DNA, 15 bp, Homo sapiens
gaatatagtgggcgt

SEQ ID NO: 258 - DNA, 15 bp, Homo sapiens
agtgggcgtagataa

SEQ ID NO: 259 - DNA, 15 bp, Homo sapiens
cgtagataaactcat

SEQ ID NO: 260 - DNA, 15 bp, Homo sapiens
taaactcataagctt

SEQ ID NO: 261 - DNA, 15 bp, Homo sapiens
cataagcttaaattc

SEQ ID NO: 262 - DNA, 15 bp, Homo sapiens
aagcttataacagat

SEQ ID NO: 263 - DNA, 15 bp, Homo sapiens
ataacagatatattt

SEQ ID NO: 264 - DNA, 15 bp, Homo sapiens
gatatattttcctgt

SEQ ID NO: 265 - DNA, 15 bp, Homo sapiens
tttcctgtctctttc

SEQ ID NO: 266 - DNA, 15 bp, Homo sapiens
atggatttttacata

SEQ ID NO: 267 - DNA, 15 bp, Homo sapiens
tgtatgcagatataa

SEQ ID NO: 268 - DNA, 15 bp, Homo sapiens
ctgtaattatgaata

SEQ ID NO: 269 - DNA, 15 bp, Homo sapiens
tacataggcaaagag

SEQ ID NO: 270 - DNA, 15 bp, Homo sapiens
caaagagttgcctgc

SEQ ID NO: 271 - DNA, 15 bp, Homo sapiens
ccagccgcttcctgc

SEQ ID NO: 272 - DNA, 15 bp, Homo sapiens
tagacatggatgaaa

SEQ ID NO: 273 - DNA, 15 bp, Homo sapiens
gatgaaattgcccct

SEQ ID NO: 274 - DNA, 15 bp, Homo sapiens
tgccccttgaatgcg

SEQ ID NO: 275 - DNA, 15 bp, Homo sapiens
tgaatgcgggtactt

SEQ ID NO: 276 - DNA, 15 bp, Homo sapiens
gtacttgaaactatt

SEQ ID NO: 277 - DNA, 15 bp, Homo sapiens
actattgcatttcgt

SEQ ID NO: 278 - DNA, 15 bp, Homo sapiens
cgttctccggtcctg

SEQ ID NO: 279 - DNA, 15 bp, Homo sapiens
ctgtgatgtgaatgc

SEQ ID NO: 280 - DNA, 15 bp, Homo sapiens
gttcgaggattagac

SEQ ID NO: 281 - DNA, 15 bp, Homo sapiens
attagactgactgga

SEQ ID NO: 282 - DNA, 15 bp, Homo sapiens
actggattcattctc

SEQ ID NO: 283 - DNA, 15 bp, Homo sapiens
attctcataattcct

SEQ ID NO: 284 - DNA, 15 bp, Homo sapiens
attcctacagcacta

SEQ ID NO: 285 - DNA, 15 bp, Homo sapiens
tcatttcattccatt

SEQ ID NO: 286 - DNA, 15 bp, Homo sapiens
tccattgcctagctc

SEQ ID NO: 287 - DNA, 15 bp, Homo sapiens
accaggtcaccggtt

SEQ ID NO: 288 - DNA, 15 bp, Homo sapiens
cgcagtagcttccta

SEQ ID NO: 289 - DNA, 15 bp, Homo sapiens
caaggagttgacatt

SEQ ID NO: 290 - DNA, 15 bp, Homo sapiens
acattttgcaggact

SEQ ID NO: 291 - DNA, 15 bp, Homo sapiens
caaggaaggcgcaca

SEQ ID NO: 292 - DNA, 15 bp, Homo sapiens
attaactttgcatga

SEQ ID NO: 293 - DNA, 15 bp, Homo sapiens
tgaataatgtgagtg

SEQ ID NO: 294 - DNA, 15 bp, Homo sapiens
gtaatttggctagag

SEQ ID NO: 295 - DNA, 15 bp, Homo sapiens
acagttccaactgtc

SEQ ID NO: 296 - protein, 23 aa, Artificial Sequence
  Synthetic Polypeptide
MWTLGRRAVAGLLASPSPAQAQT